SEQUENCE LISTING (129 sequences)

SEQ ID NO: 1 - protein, 129 aa, artificial sequence
  synthetic polypeptide
MDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYI

SEQ ID NO: 2 - protein, 92 aa, artificial sequence
  synthetic polypeptide
MSCCCSEPSDRNCTCIPITSSWAFGKFLWEWASARFNWLSLLVPFVQWFVGLSPTVWLSVIWMMWYWGPSLYSILNPFLPLLPIFFCLWVYI

SEQ ID NO: 3 - protein, 166 aa, artificial sequence
  synthetic polypeptide
MPLGFFPDHQLDPAFGANSNNPDWDFNPGGGGSGGGGSDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYI

SEQ ID NO: 4 - protein, 166 aa, artificial sequence
  synthetic polypeptide
MDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIGGGGSGGGGSPLGFFPDHQLDPAFGANSNNPDWDFNP

SEQ ID NO: 5 - protein, 159 aa, artificial sequence
  synthetic polypeptide
MDYQGMLPVCPLIPGSSTTSTGPCRTCMTTAQGTSMYPSCCCSEGGGGSPLGFFPDHQLDPAFGANSNNPDWDFNPGGGGSCIPITSSWAFGKFLWEWASARFNWLSLLVPFVQWFVGLSPTVWLSVIWMMWYWGPSLYSILNPFLPLLPIFFCLWVYI

SEQ ID NO: 6 - protein, 191 aa, artificial sequence
  synthetic polypeptide
MPLGFFPDHQLDPAFGANSNNPDWDFNPGGGGSDYQGMLPVCPLIPGSSTTSTGPCRTCMTTAQGTSMYPSCCCSEGGGGSPLGFFPDHQLDPAFGANSNNPDWDFNPGGGGSCIPITSSWAFGKFLWEWASARFNWLSLLVPFVQWFVGLSPTVWLSVIWMMWYWGPSLYSILNPFLPLLPIFFCLWVYI

SEQ ID NO: 7 - protein, 191 aa, artificial sequence
  synthetic polypeptide
MDYQGMLPVCPLIPGSSTTSTGPCRTCMTTAQGTSMYPSCCCSEGGGGSPLGFFPDHQLDPAFGANSNNPDWDFNPGGGGSCIPITSSWAFGKFLWEWASARFNWLSLLVPFVQWFVGLSPTVWLSVIWMMWYWGPSLYSILNPFLPLLPIFFCLWVYIGGGGSPLGFFPDHQLDPAFGANSNNPDWDFNP

SEQ ID NO: 8 - protein, 192 aa, artificial sequence
  synthetic polypeptide
MPLGFFPDHQLDPAFGANSNNPDWDFNPGGGGSGGGGSDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIAAAASGDSRVCWEDSWGGEVCFRYDP

SEQ ID NO: 9 - protein, 110 aa, artificial sequence
  synthetic polypeptide
MPLGFFPDHQLDPAFGANSNNPDWDFNPGGGGSGGGGSSWAFGKFLWEWASARFNWLSLLVPFVQWFVGLSPTVWLSVIWMMWYWGPSLYSILNPFLPLLPIFFCLWVYI

SEQ ID NO: 10 - protein, 111 aa, artificial sequence
  synthetic polypeptide
MSSWAFGKFLWEWASARFNWLSLLVPFVQWFVGLSPTVWLSVIWMMWYWGPSLYSILNPFLPLLPIFFCLWVYIGGGGSGGGGSPLGFFPDHQLDPAFGANSNNPDWDFNP

SEQ ID NO: 11 - protein, 137 aa, artificial sequence
  synthetic polypeptide
MPLGFFPDHQLDPAFGANSNNPDWDFNPGGGGSSWAFGKFLWEWASARFNWLSLLVPFVQWFVGLSPTVWLSVIWMMWYWGPSLYSILNPFLPLLPIFFCLWVYIGGGGSPLGFFPDHQLDPAFGANSNNPDWDFNP

SEQ ID NO: 12 - protein, 177 aa, artificial sequence
  synthetic polypeptide
MPNNNKILVPKVSGLQYRVFRGGGGSDYQGMLPVCPLIPGSSTTSTGPCRTCMTTAQGTSMYPSCCCSEGGGGSLYIKGSGSTANLASSNYFPTGGGGSCIPITSSWAFGKFLWEWASARFNWLSLLVPFVQWFVGLSPTVWLSVIWMMWYWGPSLYSILNPFLPLLPIFFCLWVYI

SEQ ID NO: 13 - protein, 123 aa, artificial sequence
  synthetic polypeptide
MPNNNKILVPKVSGLQYRVFRGGGGSSWAFGKFLWEWASARFNWLSLLVPFVQWFVGLSPTVWLSVIWMMWYWGPSLYSILNPFLPLLPIFFCLWVYIGGGGSLYIKGSGSTANLASSNYFPT

SEQ ID NO: 14 - protein, 148 aa, artificial sequence
  synthetic polypeptide
MGRGDSPGGGGSDYQGMLPVCPLIPGSSTTSTGPCRTCMTTAQGTSMYPSCCCSEGGGGSPHSRNGGGGSCIPITSSWAFGKFLWEWASARFNWLSLLVPFVQWFVGLSPTVWLSVIWMMWYWGPSLYSILNPFLPLLPIFFCLWVYI

SEQ ID NO: 15 - protein, 94 aa, artificial sequence
  synthetic polypeptide
MGRGDSPGGGGSSWAFGKFLWEWASARFNWLSLLVPFVQWFVGLSPTVWLSVIWMMWYWGPSLYSILNPFLPLLPIFFCLWVYIGGGGSPHSRN

SEQ ID NO: 16 - protein, 167 aa, artificial sequence
  synthetic polypeptide
MQLPLATGGGGSDYQGMLPVCPLIPGSSTTSTGPCRTCMTTAQGTSMYPSCCCSEGGGGSMQLPLATGGGGSCIPITSSWAFGKFLWEWASARFNWLSLLVPFVQWFVGLSPTVWLSVIWMMWYWGPSLYSILNPFLPLLPIFFCLWVYIGGGGSGGGGSMQLPLAT

SEQ ID NO: 17 - protein, 96 aa, artificial sequence
  synthetic polypeptide
MQLPLATGGGGSSWAFGKFLWEWASARFNWLSLLVPFVQWFVGLSPTVWLSVIWMMWYWGPSLYSILNPFLPLLPIFFCLWVYIGGGGSMQLPLAT

SEQ ID NO: 18 - DNA, 438 bp, artificial sequence
  synthetic oligonucleotide
atggactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcgactacaaggacgacgacgacaaggtcgaccaccaccaccaccaccactga

SEQ ID NO: 19 - DNA, 438 bp, artificial sequence
  synthetic oligonucleotide
atggactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgactacaaggacgacgacgacaaggtcgaccaccaccaccaccaccactga

SEQ ID NO: 20 - DNA, 327 bp, artificial sequence
  synthetic oligonucleotide
atgtcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcgactacaaggacgacgacgacaaggtcgaccaccaccaccaccaccactga

SEQ ID NO: 21 - DNA, 327 bp, artificial sequence
  synthetic oligonucleotide
atgtcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgactacaaggacgacgacgacaaggtcgaccaccaccaccaccaccactga

SEQ ID NO: 22 - DNA, 501 bp, artificial sequence
  synthetic oligonucleotide
atgccattgggtttcttcccagaccaccaattggacccagctttcggtgctaactctaacaacccagactgggacttcaacccaggtggtggtggttctggtggtggtggttctgactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatctga

SEQ ID NO: 23 - DNA, 501 bp, artificial sequence
  synthetic oligonucleotide
atgcccctgggcttcttccccgaccaccagctggaccccgccttcggcgccaacagcaacaaccccgactgggacttcaaccccggcggcggcggcagcggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatctga

SEQ ID NO: 24 - DNA, 501 bp, artificial sequence
  synthetic oligonucleotide
atggactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctccattgggtttcttcccagaccaccaattggacccagctttcggtgctaactctaacaacccagactgggacttcaacccatga

SEQ ID NO: 25 - DNA, 501 bp, artificial sequence
  synthetic oligonucleotide
atggactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccccctgggcttcttccccgaccaccagctggaccccgccttcggcgccaacagcaacaaccccgactgggacttcaacccctga

SEQ ID NO: 26 - DNA, 480 bp, artificial sequence
  synthetic oligonucleotide
atggactaccaaggtatgttgccagtttgtccattgatcccaggttcttctactacttctactggtccatgtagaacttgtatgactactgctcaaggtacttctatgtacccatcttgttgttgttctgaaggtggtggtggttctccattgggtttcttcccagaccaccaattggacccagctttcggtgctaactctaacaacccagactgggacttcaacccaggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatctga

SEQ ID NO: 27 - DNA, 480 bp, artificial sequence
  synthetic oligonucleotide
atggactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagccccctgggcttcttccccgaccaccagctggaccccgccttcggcgccaacagcaacaaccccgactgggacttcaaccccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatctga

SEQ ID NO: 28 - DNA, 576 bp, artificial sequence
  synthetic oligonucleotide
atgccattgggtttcttcccagaccaccaattggacccagctttcggtgctaactctaacaacccagactgggacttcaacccaggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctccattgggtttcttcccagaccaccaattggacccagctttcggtgctaactctaacaacccagactgggacttcaacccaggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatctga

SEQ ID NO: 29 - DNA, 576 bp, artificial sequence
  synthetic oligonucleotide
atgcccctgggcttcttccccgaccaccagctggaccccgccttcggcgccaacagcaacaaccccgactgggacttcaaccccggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagccccctgggcttcttccccgaccaccagctggaccccgccttcggcgccaacagcaacaaccccgactgggacttcaaccccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatctga

SEQ ID NO: 30 - DNA, 576 bp, artificial sequence
  synthetic oligonucleotide
atggactaccaaggtatgttgccagtttgtccattgatcccaggttcttctactacttctactggtccatgtagaacttgtatgactactgctcaaggtacttctatgtacccatcttgttgttgttctgaaggtggtggtggttctccattgggtttcttcccagaccaccaattggacccagctttcggtgctaactctaacaacccagactgggacttcaacccaggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctccattgggtttcttcccagaccaccaattggacccagctttcggtgctaactctaacaacccagactgggacttcaacccatga

SEQ ID NO: 31 - DNA, 576 bp, artificial sequence
  synthetic oligonucleotide
atggactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagccccctgggcttcttccccgaccaccagctggaccccgccttcggcgccaacagcaacaaccccgactgggacttcaaccccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagccccctgggcttcttccccgaccaccagctggaccccgccttcggcgccaacagcaacaaccccgactgggacttcaacccctga

SEQ ID NO: 32 - DNA, 579 bp, artificial sequence
  synthetic oligonucleotide
atgccattgggtttcttcccagaccaccaattggacccagctttcggtgctaactctaacaacccagactgggacttcaacccaggtggtggtggttctggtggtggtggttctgactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcgctgctgctgcttctggtgactctagagtttgttgggaagactcttggggtggtgaagtttgtttcagatacgacccatga

SEQ ID NO: 33 - DNA, 579 bp, artificial sequence
  synthetic oligonucleotide
atgcccctgggcttcttccccgaccaccagctggaccccgccttcggcgccaacagcaacaaccccgactgggacttcaaccccggcggcggcggcagcggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgccgccgccgccagcggcgacagccgcgtgtgctgggaggacagctggggcggcgaggtgtgcttccgctacgacccctga

SEQ ID NO: 34 - DNA, 333 bp, artificial sequence
  synthetic oligonucleotide
atgccattgggtttcttcccagaccaccaattggacccagctttcggtgctaactctaacaacccagactgggacttcaacccaggtggtggtggttctggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatctga

SEQ ID NO: 35 - DNA, 333 bp, artificial sequence
  synthetic oligonucleotide
atgcccctgggcttcttccccgaccaccagctggaccccgccttcggcgccaacagcaacaaccccgactgggacttcaaccccggcggcggcggcagcggcggcggcggctcctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatctga

SEQ ID NO: 36 - DNA, 336 bp, artificial sequence
  synthetic oligonucleotide
atgtcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctccattgggtttcttcccagaccaccaattggacccagctttcggtgctaactctaacaacccagactgggacttcaacccatga

SEQ ID NO: 37 - DNA, 336 bp, artificial sequence
  synthetic oligonucleotide
atgagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccccctgggcttcttccccgaccaccagctggaccccgccttcggcgccaacagcaacaaccccgactgggacttcaacccctga

SEQ ID NO: 38 - DNA, 429 bp, artificial sequence
  synthetic oligonucleotide
atgccattgggtttcttcccagaccaccaattggacccagctttcggtgctaactctaacaacccagactgggacttcaacccaggtggtggtggttctggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctccattgggtttcttcccagaccaccaattggacccagctttcggtgctaactctaacaacccagactgggacttcaacccatga

SEQ ID NO: 39 - DNA, 429 bp, artificial sequence
  synthetic oligonucleotide
atgcccctgggcttcttccccgaccaccagctggaccccgccttcggcgccaacagcaacaaccccgactgggacttcaaccccggcggcggcggcagcggcggcggcggctcctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagccccctgggcttcttccccgaccaccagctggaccccgccttcggcgccaacagcaacaaccccgactgggacttcaacccctga

SEQ ID NO: 40 - DNA, 534 bp, artificial sequence
  synthetic oligonucleotide
atgccaaacaacaacaagatcttggttccaaaggtttctggtttgcaatacagagttttcagaggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctttgtacatcaagggttctggttctactgctaacttggcttcttctaactacttcccaactggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatctga

SEQ ID NO: 41 - DNA, 534 bp, artificial sequence
  synthetic oligonucleotide
atgcccaacaacaacaagatcctggtgcccaaggtgagcggcctgcagtaccgcgtgttccgcggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagcctgtacatcaagggcagcggcagcaccgccaacctggccagcagcaactacttccccaccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatctga

SEQ ID NO: 42 - DNA, 372 bp, artificial sequence
  synthetic oligonucleotide
atgccaaacaacaacaagatcttggttccaaaggtttctggtttgcaatacagagttttcagaggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctttgtacatcaagggttctggttctactgctaacttggcttcttctaactacttcccaacttga

SEQ ID NO: 43 - DNA, 372 bp, artificial sequence
  synthetic oligonucleotide
atgcccaacaacaacaagatcctggtgcccaaggtgagcggcctgcagtaccgcgtgttccgcggcggcggcggcagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcctgtacatcaagggcagcggcagcaccgccaacctggccagcagcaactacttccccacctga

SEQ ID NO: 44 - DNA, 447 bp, artificial sequence
  synthetic oligonucleotide
atgggtagaggtgactctccaggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctccacactctagaaacggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatctga

SEQ ID NO: 45 - DNA, 447 bp, artificial sequence
  synthetic oligonucleotide
atgggcgcgggcgacagccccggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagcccccacagccgcaacggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatctga

SEQ ID NO: 46 - DNA, 285 bp, artificial sequence
  synthetic oligonucleotide
atgggtagaggtgactctccaggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctccacactctagaaactga

SEQ ID NO: 47 - DNA, 285 bp, artificial sequence
  synthetic oligonucleotide
atgggccgcggcgacagccccggcggcggcggcagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcccccacagccgcaactga

SEQ ID NO: 48 - DNA, 501 bp, artificial sequence
  synthetic oligonucleotide
atgcaattgccattggctactggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggtatgcaattgccattggctactggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctatgcaattgccattggctacttga

SEQ ID NO: 49 - DNA, 501 bp, artificial sequence
  synthetic oligonucleotide
atgcagctccccctcgccaccggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcatgcagctgcccctggccaccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagcatgcagctgcccctggccacctga

SEQ ID NO: 50 - DNA, 291 bp, artificial sequence
  synthetic oligonucleotide
atgcaattgccattggctactggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctatgcaattgccattggctacttga

SEQ ID NO: 51 - DNA, 291 bp, artificial sequence
  synthetic oligonucleotide
atgcagctgcccctggccaccggcggcggcggcagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcatgcagctgcccctggccacctga

SEQ ID NO: 52 - protein, 8 aa, artificial sequence
  synthetic polypeptide, affinity tag
DYKDDDDK

SEQ ID NO: 53 - protein, 6 aa, artificial sequence
  synthetic polypeptide, affinity tag
HHHHHH

SEQ ID NO: 54 - protein, 27 aa, artificial sequence
  synthetic polypeptide
PLGFFPDHQLDPAFGANSNNPDWDFNP

SEQ ID NO: 55 - protein, 5 aa, artificial sequence
  synthetic polypeptide, linker
GGGGS

SEQ ID NO: 56 - protein, 5 aa, artificial sequence
  synthetic polypeptide, linker
AAAAS

SEQ ID NO: 57 - protein, 145 aa, artificial sequence
  synthetic polypeptide
MDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIDYKDDDDKVDHHHHHH

SEQ ID NO: 58 - protein, 108 aa, artificial sequence
  synthetic polypeptide
MSCCCSEPSDRNCTCIPITSSWAFGKFLWEWASARFNWLSLLVPFVQWFVGLSPTVWLSVIWMMWYWGPSLYSILNPFLPLLPIFFCLWVYIDYKDDDDKVDHHHHHH

SEQ ID NO: 59 - protein, 22 aa, artificial sequence
  synthetic polypeptide
SGDSRVCWEDSWGGEVCFRYDP

SEQ ID NO: 60 - protein, 165 aa, artificial sequence
  synthetic polypeptide
METDTLLLWVLLLWVPGSTGDDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIDYKDDDDKVDHHHHHH

SEQ ID NO: 61 - protein, 20 aa, artificial sequence
  synthetic polypeptide
PNNNKILVPKVSGLQYRVFR

SEQ ID NO: 62 - protein, 20 aa, artificial sequence
  synthetic polypeptide
LYIKGSGSTANLASSNYFPT

SEQ ID NO: 63 - protein, 128 aa, artificial sequence
  synthetic polypeptide
METDTLLLWVLLLWVPGSTGDSCCCSEPSDRNCTCIPITSSWAFGKFLWEWASARFNWLSLLVPFVQWFVGLSPTVWLSVIWMMWYWGPSLYSILNPFLPLLPIFFCLWVYIDYKDDDDKVDHHHHHH

SEQ ID NO: 64 - protein, 6 aa, artificial sequence
  synthetic polypeptide
GRGDSP

SEQ ID NO: 65 - protein, 5 aa, artificial sequence
  synthetic polypeptide
PHSRN

SEQ ID NO: 66 - protein, 7 aa, artificial sequence
  synthetic polypeptide
MQLPLAT

SEQ ID NO: 67 - protein, 226 aa, artificial sequence
  synthetic polypeptide
METDTLLLWVLLLWVPGSTGDPLGFFPDHQLDPAFGANSNNPDWDFNPGGGGSGGGGSDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIGGGGSGGGGSRLATELKSLNPRWDGERLYQEARKIVGAMV

SEQ ID NO: 68 - protein, 226 aa, artificial sequence
  synthetic polypeptide
METDTLLLWVLLLWVPGSTGDGGGGSGGGGSRLATELKSLNPRWDGERLYQEARKIVGAMVDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIGGGGSGGGGSPLGFFPDHQLDPAFGANSNNPDWDFNP

SEQ ID NO: 69 - protein, 219 aa, artificial sequence
  synthetic polypeptide
METDTLLLWVLLLWVPGSTGDDYQGMLPVCPLIPGSSTTSTGPCRTCMTTAQGTSMYPSCCCSEGGGGSPLGFFPDHQLDPAFGANSNNPDWDFNPGGGGSCIPITSSWAFGKFLWEWASARFNWLSLLVPFVQWFVGLSPTVWLSVIWMMWYWGPSLYSILNPFLPLLPIFFCLWVYIGGGGSGGGGSRLATELKSLNPRWDGERLYQEARKIVGAMV

SEQ ID NO: 70 - protein, 30 aa, artificial sequence
  synthetic polypeptide
RLATELKSLNPRWDGERLYQEARKIVGAMV

SEQ ID NO: 71 - protein, 21 aa, artificial sequence
  synthetic polypeptide
METDTLLLWVLLLWVPGSTGD

SEQ ID NO: 72 - protein, 21 aa, artificial sequence
  synthetic polypeptide
MAMKVLVFFVATILVAWQCHT

SEQ ID NO: 73 - protein, 251 aa, artificial sequence
  synthetic polypeptide
METDTLLLWVLLLWVPGSTGDPLGFFPDHQLDPAFGANSNNPDWDFNPGGGGSDYQGMLPVCPLIPGSSTTSTGPCRTCMTTAQGTSMYPSCCCSEGGGGSPLGFFPDHQLDPAFGANSNNPDWDFNPGGGGSCIPITSSWAFGKFLWEWASARFNWLSLLVPFVQWFVGLSPTVWLSVIWMMWYWGPSLYSILNPFLPLLPIFFCLWVYIGGGGSGGGGSRLATELKSLNPRWDGERLYQEARKIVGAMV

SEQ ID NO: 74 - protein, 251 aa, artificial sequence
  synthetic polypeptide
METDTLLLWVLLLWVPGSTGDGGGGSGGGGSRLATELKSLNPRWDGERLYQEARKIVGAMVDYQGMLPVCPLIPGSSTTSTGPCRTCMTTAQGTSMYPSCCCSEGGGGSPLGFFPDHQLDPAFGANSNNPDWDFNPGGGGSCIPITSSWAFGKFLWEWASARFNWLSLLVPFVQWFVGLSPTVWLSVIWMMWYWGPSLYSILNPFLPLLPIFFCLWVYIGGGGSPLGFFPDHQLDPAFGANSNNPDWDFNP

SEQ ID NO: 75 - protein, 252 aa, artificial sequence
  synthetic polypeptide
METDTLLLWVLLLWVPGSTGDPLGFFPDHQLDPAFGANSNNPDWDFNPGGGGSGGGGSDYQGMLPVCPLIPGSSTTSTGPCRTCMTTAQGTSMYPSCCCSEPSDRNCTCIPITSSWAFGKFLWEWASARFNWLSLLVPFVQWFVGLSPTVWLSVIWMMWYWGPSLYSILNPFLPLLPIFFCLWVYIAAAASGDSRVCWEDSWGGEVCFRYDPGGGGSGGGGSRLATELKSLNPRWDGERLYQEARKIVGAMV

SEQ ID NO: 76 - protein, 170 aa, artificial sequence
  synthetic polypeptide
METDTLLLWVLLLWVPGSTGDPLGFFPDHQLDPAFGANSNNPDWDFNPGGGGSGGGGSSWAFGKFLWEWASARFNWLSLLVPFVQWFVGLSPTVWLSVIWMMWYWGPSLYSILNPFLPLLPIFFCLWVYIGGGGSGGGGSRLATELKSLNPRWDGERLYQEARKIVGAMV

SEQ ID NO: 77 - protein, 171 aa, artificial sequence
  synthetic polypeptide
METDTLLLWVLLLWVPGSTGDGGGGSGGGGSRLATELKSLNPRWDGERLYQEARKIVGAMVSSWAFGKFLWEWASARFNWLSLLVPFVQWFVGLSPTVWLSVIWMMWYWGPSLYSILNPFLPLLPIFFCLWVYIGGGGSGGGGSPLGFFPDHQLDPAFGANSNNPDWDFNP

SEQ ID NO: 78 - protein, 167 aa, artificial sequence
  synthetic polypeptide
METDTLLLWVLLLWVPGSTGDPLGFFPDHQLDPAFGANSNNPDWDFNPGGGGSGGGGSSWAFGKFLWEWASARFNWLSLLVPFVQWFVGLSPTVWLSVIWMMWYWGPSLYSILNPFLPLLPIFFCLWVYIGGGGSGGGGSPLGFFPDHQLDPAFGANSNNPDWDFNP

SEQ ID NO: 79 - protein, 237 aa, artificial sequence
  synthetic polypeptide
METDTLLLWVLLLWVPGSTGDPNNNKILVPKVSGLQYRVFRGGGGSDYQGMLPVCPLIPGSSTTSTGPCRTCMTTAQGTSMYPSCCCSEGGGGSLYIKGSGSTANLASSNYFPTGGGGSCIPITSSWAFGKFLWEWASARFNWLSLLVPFVQWFVGLSPTVWLSVIWMMWYWGPSLYSILNPFLPLLPIFFCLWVYIGGGGSGGGGSRLATELKSLNPRWDGERLYQEARKIVGAMV

SEQ ID NO: 80 - protein, 143 aa, artificial sequence
  synthetic polypeptide
METDTLLLWVLLLWVPGSTGDPNNNKILVPKVSGLQYRVFRGGGGSSWAFGKFLWEWASARFNWLSLLVPFVQWFVGLSPTVWLSVIWMMWYWGPSLYSILNPFLPLLPIFFCLWVYIGGGGSLYIKGSGSTANLASSNYFPT

SEQ ID NO: 81 - protein, 208 aa, artificial sequence
  synthetic polypeptide
METDTLLLWVLLLWVPGSTGDGRGDSPGGGGSDYQGMLPVCPLIPGSSTTSTGPCRTCMTTAQGTSMYPSCCCSEGGGGSPHSRNGGGGSCIPITSSWAFGKFLWEWASARFNWLSLLVPFVQWFVGLSPTVWLSVIWMMWYWGPSLYSILNPFLPLLPIFFCLWVYIGGGGSGGGGSRLATELKSLNPRWDGERLYQEARKIVGAMV

SEQ ID NO: 82 - protein, 154 aa, artificial sequence
  synthetic polypeptide
METDTLLLWVLLLWVPGSTGDGRGDSPGGGGSRLATELKSLNPRWDGERLYQEARKIVGAMVGGGGSGGGGSSWAFGKFLWEWASARFNWLSLLVPFVQWFVGLSPTVWLSVIWMMWYWGPSLYSILNPFLPLLPIFFCLWVYIGGGGSPHSRN

SEQ ID NO: 83 - protein, 187 aa, artificial sequence
  synthetic polypeptide
METDTLLLWVLLLWVPGSTGDQLPLATGGGGSDYQGMLPVCPLIPGSSTTSTGPCRTCMTTAQGTSMYPSCCCSEGGGGSMQLPLATGGGGSCIPITSSWAFGKFLWEWASARFNWLSLLVPFVQWFVGLSPTVWLSVIWMMWYWGPSLYSILNPFLPLLPIFFCLWVYIGGGGSGGGGSMQLPLAT

SEQ ID NO: 84 - protein, 116 aa, artificial sequence
  synthetic polypeptide
METDTLLLWVLLLWVPGSTGDQLPLATGGGGSSWAFGKFLWEWASARFNWLSLLVPFVQWFVGLSPTVWLSVIWMMWYWGPSLYSILNPFLPLLPIFFCLWVYIGGGGSMQLPLAT

SEQ ID NO: 85 - DNA, 498 bp, artificial sequence
  synthetic oligonucleotide
atggaaactgacactttgttgttgtgggttttgttgttgtgggttccaggttctactggtgacgactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcgactacaaggacgacgacgacaaggtcgaccaccaccaccaccaccactga

SEQ ID NO: 86 - DNA, 498 bp, artificial sequence
  synthetic oligonucleotide
atggagaccgacaccctgctgctgtgggtgctgctgctgtgggtgcccggcagcaccggcgacgactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgactacaaggacgacgacgacaaggtcgaccaccaccaccaccaccactga

SEQ ID NO: 87 - DNA, 387 bp, artificial sequence
  synthetic oligonucleotide
atggaaactgacactttgttgttgtgggttttgttgttgtgggttccaggttctactggtgactcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcgactacaaggacgacgacgacaaggtcgaccaccaccaccaccaccactga

SEQ ID NO: 88 - DNA, 387 bp, artificial sequence
  synthetic oligonucleotide
atggagaccgacaccctgctgctgtgggtgctgctgctgtgggtgcccggcagcaccggcgactcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgactacaaggacgacgacgacaaggtcgaccaccaccaccaccaccactga

SEQ ID NO: 89 - DNA, 681 bp, artificial sequence
  synthetic oligonucleotide
atggaaactgacactttgttgttgtgggttttgttgttgtgggttccaggttctactggtgacccattgggtttcttcccagaccaccaattggacccagctttcggtgctaactctaacaacccagactgggacttcaacccaggtggtggtggttctggtggtggtggttctgactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctagattggctactgaattgaagtctttgaacccaagatgggacggtgaaagattgtaccaagaagctagaaagatcgttggtgctatggtttga

SEQ ID NO: 90 - DNA, 681 bp, artificial sequence
  synthetic oligonucleotide
atggagaccgacaccctgctgctgtgggtgctgctgctgtgggtgcccggcagcaccggcgaccccctgggcttcttccccgaccaccagctggaccccgccttcggcgccaacagcaacaaccccgactgggacttcaaccccggcggcggcggcagcggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 91 - DNA, 681 bp, artificial sequence
  synthetic oligonucleotide
atggaaactgacactttgttgttgtgggttttgttgttgtgggttccaggttctactggtgacggtggtggtggttctggtggtggtggttctagattggctactgaattgaagtctttgaacccaagatgggacggtgaaagattgtaccaagaagctagaaagatcgttggtgctatggttgactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctccattgggtttcttcccagaccaccaattggacccagctttcggtgctaactctaacaacccagactgggacttcaacccatga

SEQ ID NO: 92 - DNA, 681 bp, artificial sequence
  synthetic oligonucleotide
atggagaccgacaccctgctgctgtgggtgctgctgctgtgggtgcccggcagcaccggcgacggcggcggcggcagcggcggcggcggcagccgcctggccaccgagctgaagagcctgaacccccgctgggacggcgagcgcctgtaccaggaggcccgcaagatcgtgggcgccatggtggactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccccctgggcttcttccccgaccaccagctggaccccgccttcggcgccaacagcaacaaccccgactgggacttcaacccctga

SEQ ID NO: 93 - DNA, 660 bp, artificial sequence
  synthetic oligonucleotide
atggaaactgacactttgttgttgtgggttttgttgttgtgggttccaggttctactggtgacgactaccaaggtatgttgccagtttgtccattgatcccaggttcttctactacttctactggtccatgtagaacttgtatgactactgctcaaggtacttctatgtacccatcttgttgttgttctgaaggtggtggtggttctccattgggtttcttcccagaccaccaattggacccagctttcggtgctaactctaacaacccagactgggacttcaacccaggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctagattggctactgaattgaagtctttgaacccaagatgggacggtgaaagattgtaccaagaagctagaaagatcgttggtgctatggtttga

SEQ ID NO: 94 - DNA, 660 bp, artificial sequence
  synthetic oligonucleotide
atggagaccgacaccctgctgctgtgggtgctgctgctgtgggtgcccggcagcaccggcgacgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagccccctgggcttcttccccgaccaccagctggaccccgccttcggcgccaacagcaacaaccccgactgggacttcaaccccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 95 - DNA, 756 bp, artificial sequence
  synthetic oligonucleotide
atggaaactgacactttgttgttgtgggttttgttgttgtgggttccaggttctactggtgacccattgggtttcttcccagaccaccaattggacccagctttcggtgctaactctaacaacccagactgggacttcaacccaggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctccattgggtttcttcccagaccaccaattggacccagctttcggtgctaactctaacaacccagactgggacttcaacccaggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctagattggctactgaattgaagtctttgaacccaagatgggacggtgaaagattgtaccaagaagctagaaagatcgttggtgctatggtttga

SEQ ID NO: 96 - DNA, 756 bp, artificial sequence
  synthetic oligonucleotide
atggagaccgacaccctgctgctgtgggtgctgctgctgtgggtgcccggcagcaccggcgaccccctgggcttcttccccgaccaccagctggaccccgccttcggcgccaacagcaacaaccccgactgggacttcaaccccggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagccccctgggcttcttccccgaccaccagctggaccccgccttcggcgccaacagcaacaaccccgactgggacttcaaccccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 97 - DNA, 756 bp, artificial sequence
  synthetic oligonucleotide
atggaaactgacactttgttgttgtgggttttgttgttgtgggttccaggttctactggtgacggtggtggtggttctggtggtggtggttctagattggctactgaattgaagtctttgaacccaagatgggacggtgaaagattgtaccaagaagctagaaagatcgttggtgctatggttgactaccaaggtatgttgccagtttgtccattgatcccaggttcttctactacttctactggtccatgtagaacttgtatgactactgctcaaggtacttctatgtacccatcttgttgttgttctgaaggtggtggtggttctccattgggtttcttcccagaccaccaattggacccagctttcggtgctaactctaacaacccagactgggacttcaacccaggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctccattgggtttcttcccagaccaccaattggacccagctttcggtgctaactctaacaacccagactgggacttcaacccatga

SEQ ID NO: 98 - DNA, 756 bp, artificial sequence
  synthetic oligonucleotide
atggagaccgacaccctgctgctgtgggtgctgctgctgtgggtgcccggcagcaccggcgacggcggcggcggcagcggcggcggcggcagccgcctggccaccgagctgaagagcctgaacccccgctgggacggcgagcgcctgtaccaggaggcccgcaagatcgtgggcgccatggtggactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagccccctgggcttcttccccgaccaccagctggaccccgccttcggcgccaacagcaacaaccccgactgggacttcaaccccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagccccctgggcttcttccccgaccaccagctggaccccgccttcggcgccaacagcaacaaccccgactgggacttcaacccctga

SEQ ID NO: 99 - DNA, 759 bp, artificial sequence
  synthetic oligonucleotide
atggaaactgacactttgttgttgtgggttttgttgttgtgggttccaggttctactggtgacccattgggtttcttcccagaccaccaattggacccagctttcggtgctaactctaacaacccagactgggacttcaacccaggtggtggtggttctggtggtggtggttctgactaccaaggtatgttgccagtttgtccattgatcccaggttcttctactacttctactggtccatgtagaacttgtatgactactgctcaaggtacttctatgtacccatcttgttgttgttctgaaccatctgacagaaactgtacttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcgctgctgctgcttctggtgactctagagtttgttgggaagactcttggggtggtgaagtttgtttcagatacgacccaggtggtggtggttctggtggtggtggttctagattggctactgaattgaagtctttgaacccaagatgggacggtgaaagattgtaccaagaagctagaaagatcgttggtgctatggtttga

SEQ ID NO: 100 - DNA, 759 bp, artificial sequence
  synthetic oligonucleotide
atggagaccgacaccctgctgctgtgggtgctgctgctgtgggtgcccggcagcaccggcgaccccctgggcttcttccccgaccaccagctggaccccgccttcggcgccaacagcaacaaccccgactgggacttcaaccccggcggcggcggcagcggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagcccagcgaccgcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgccgccgccgccagcggcgacagccgcgtgtgctgggaggacagctggggcggcgaggtgtgcttccgctacgaccccggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 101 - DNA, 513 bp, artificial sequence
  synthetic oligonucleotide
atggaaactgacactttgttgttgtgggttttgttgttgtgggttccaggttctactggtgacccattgggtttcttcccagaccaccaattggacccagctttcggtgctaactctaacaacccagactgggacttcaacccaggtggtggtggttctggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctagattggctactgaattgaagtctttgaacccaagatgggacggtgaaagattgtaccaagaagctagaaagatcgttggtgctatggtttga

SEQ ID NO: 102 - DNA, 513 bp, artificial sequence
  synthetic oligonucleotide
atggagaccgacaccctgctgctgtgggtgctgctgctgtgggtgcccggcagcaccggcgaccccctgggcttcttccccgaccaccagctggaccccgccttcggcgccaacagcaacaaccccgactgggacttcaaccccggcggcggcggcagcggcggcggcggctcctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 103 - DNA, 516 bp, artificial sequence
  synthetic oligonucleotide
atggaaactgacactttgttgttgtgggttttgttgttgtgggttccaggttctactggtgacggtggtggtggttctggtggtggtggttctagattggctactgaattgaagtctttgaacccaagatgggacggtgaaagattgtaccaagaagctagaaagatcgttggtgctatggtttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctccattgggtttcttcccagaccaccaattggacccagctttcggtgctaactctaacaacccagactgggacttcaacccatga

SEQ ID NO: 104 - DNA, 516 bp, artificial sequence
  synthetic oligonucleotide
atggagaccgacaccctgctgctgtgggtgctgctgctgtgggtgcccggcagcaccggcgacggcggcggcggcagcggcggcggcggcagccgcctggccaccgagctgaagagcctgaacccccgctgggacggcgagcgcctgtaccaggaggcccgcaagatcgtgggcgccatggtgagcagctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccccctgggcttcttccccgaccaccagctggaccccgccttcggcgccaacagcaacaaccccgactgggacttcaacccctga

SEQ ID NO: 105 - DNA, 504 bp, artificial sequence
  synthetic oligonucleotide
atggaaactgacactttgttgttgtgggttttgttgttgtgggttccaggttctactggtgacccattgggtttcttcccagaccaccaattggacccagctttcggtgctaactctaacaacccagactgggacttcaacccaggtggtggtggttctggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctccattgggtttcttcccagaccaccaattggacccagctttcggtgctaactctaacaacccagactgggacttcaacccatga

SEQ ID NO: 106 - DNA, 504 bp, artificial sequence
  synthetic oligonucleotide
atggagaccgacaccctgctgctgtgggtgctgctgctgtgggtgcccggcagcaccggcgaccccctgggcttcttccccgaccaccagctggaccccgccttcggcgccaacagcaacaaccccgactgggacttcaaccccggcggcggcggcagcggcggcggcggctcctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccccctgggcttcttccccgaccaccagctggaccccgccttcggcgccaacagcaacaaccccgactgggacttcaacccctga

SEQ ID NO: 107 - DNA, 714 bp, artificial sequence
  synthetic oligonucleotide
atggaaactgacactttgttgttgtgggttttgttgttgtgggttccaggttctactggtgacccaaacaacaacaagatcttggttccaaaggtttctggtttgcaatacagagttttcagaggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctttgtacatcaagggttctggttctactgctaacttggcttcttctaactacttcccaactggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctagattggctactgaattgaagtctttgaacccaagatgggacggtgaaagattgtaccaagaagctagaaagatcgttggtgctatggtttga

SEQ ID NO: 108 - DNA, 714 bp, artificial sequence
  synthetic oligonucleotide
atggagaccgacaccctgctgctgtgggtgctgctgctgtgggtgcccggcagcaccggcgaccccaacaacaacaagatcctggtgcccaaggtgagcggcctgcagtaccgcgtgttccgcggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagcctgtacatcaagggcagcggcagcaccgccaacctggccagcagcaactacttccccaccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 109 - DNA, 432 bp, artificial sequence
  synthetic oligonucleotide
atggaaactgacactttgttgttgtgggttttgttgttgtgggttccaggttctactggtgacccaaacaacaacaagatcttggttccaaaggtttctggtttgcaatacagagttttcagaggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctttgtacatcaagggttctggttctactgctaacttggcttcttctaactacttcccaacttga

SEQ ID NO: 110 - DNA, 432 bp, artificial sequence
  synthetic oligonucleotide
atggagaccgacaccctgctgctgtgggtgctgctgctgtgggtgcccggcagcaccggcgaccccaacaacaacaagatcctggtgcccaaggtgagcggcctgcagtaccgcgtgttccgcggcggcggcggcagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcctgtacatcaagggcagcggcagcaccgccaacctggccagcagcaactacttccccacctga

SEQ ID NO: 111 - DNA, 627 bp, artificial sequence
  synthetic oligonucleotide
atggaaactgacactttgttgttgtgggttttgttgttgtgggttccaggttctactggtgacggtagaggtgactctccaggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctccacactctagaaacggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctagattggctactgaattgaagtctttgaacccaagatgggacggtgaaagattgtaccaagaagctagaaagatcgttggtgctatggtttga

SEQ ID NO: 112 - DNA, 627 bp, artificial sequence
  synthetic oligonucleotide
atggagaccgacaccctgctgctgtgggtgctgctgctgtgggtgcccggcagcaccggcgacggccgcggcgacagccccggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagcccccacagccgcaacggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagccgcctggccaccgagctgaagagcctgaacccccgctgggacggcgagcgcctgtaccaggaggcccgcaagatcgtgggcgccatggtgtga

SEQ ID NO: 113 - DNA, 465 bp, artificial sequence
  synthetic oligonucleotide
atggaaactgacactttgttgttgtgggttttgttgttgtgggttccaggttctactggtgacggtagaggtgactctccaggtggtggtggttctagattggctactgaattgaagtctttgaacccaagatgggacggtgaaagattgtaccaagaagctagaaagatcgttggtgctatggttggtggtggtggttctggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctccacactctagaaactga

SEQ ID NO: 114 - DNA, 465 bp, artificial sequence
  synthetic oligonucleotide
atggagaccgacaccctgctgctgtgggtgctgctgctgtgggtgcccggcagcaccggcgacggccgcggcgacagccccggcggcggcggcagccgcctggccaccgagctgaagagcctgaacccccgctgggacggcgagcgcctgtaccaggaggcccgcaagatcgtgggcgccatggtgggcggcggcggcagcggcggcggcggcagcagctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcccccacagccgcaactga

SEQ ID NO: 115 - DNA, 564 bp, artificial sequence
  synthetic oligonucleotide
atggaaactgacactttgttgttgtgggttttgttgttgtgggttccaggttctactggtgaccaattgccattggctactggtggtggtggttctgactaccaaggtatgttgccagtttgtccattgatcccaggttcttctactacttctactggtccatgtagaacttgtatgactactgctcaaggtacttctatgtacccatcttgttgttgttctgaaggtggtggtggttctatgcaattgccattggctactggtggtggtggttcttgtatcccaatcacttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctggtggtggtggttctatgcaattgccattggctacttga

SEQ ID NO: 116 - DNA, 564 bp, artificial sequence
  synthetic oligonucleotide
atggagaccgacaccctgctgctgtgggtgctgctgctgtgggtgcccggcagcaccggcgaccagctccccctcgccaccggcggcggcggcagcgactaccagggcatgctgcccgtgtgccccctgatccccggctccagcaccaccagcaccggcccctgccgcacctgcatgaccaccgcccagggcacctccatgtacccctcctgctgctgcagcgagggcggcggcggcagcatgcagctgcccctggccaccggcggcggcggcagctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcggcggcggcggcagcatgcagctgcccctggccacctga

SEQ ID NO: 117 - DNA, 351 bp, artificial sequence
  synthetic oligonucleotide
atggaaactgacactttgttgttgtgggttttgttgttgtgggttccaggttctactggtgaccaattgccattggctactggtggtggtggttcttcttgggctttcggtaagttcttgtgggaatgggcttctgctagattcaactggttgtctttgttggttccattcgttcaatggttcgttggtttgtctccaactgtttggttgtctgttatctggatgatgtggtactggggtccatctttgtactctatcttgaacccattcttgccattgttgccaatcttcttctgtttgtgggtttacatcggtggtggtggttctatgcaattgccattggctacttga

SEQ ID NO: 118 - DNA, 351 bp, artificial sequence
  synthetic oligonucleotide
atggagaccgacaccctgctgctgtgggtgctgctgctgtgggtgcccggcagcaccggcgaccagctgcccctggccaccggcggcggcggcagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcggcggcggcggcagcatgcagctgcccctggccacctga

SEQ ID NO: 119 - DNA, 729 bp, artificial sequence
  synthetic oligonucleotide
atggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacattgactacaaggacgacgacgacaaggtcgaccaccaccaccaccaccactga

SEQ ID NO: 120 - protein, 242 aa, artificial sequence
  synthetic polypeptide
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYIDYKDDDDKVDHHHHHH

SEQ ID NO: 121 - DNA, 729 bp, artificial sequence
  synthetic oligonucleotide
atggagaacatcacctccggcttcctgggccccctgctggtgctgcaggccggcttcttcctgctgacccgcatcctgaccatcccccagagcctggactcctggtggacctccctgaacttcctgggcggcaccaccgtgtgcctgggccagaactcccagtcccccacctccaaccactcccccacctcctgcccccccacctgccccggctaccgctggatgtgcctgcgccgcttcatcatcttcctgttcatcctgctgctgtgcctgatcttcctgctggtgctgctggactaccagggcatgctgcccgtgtgccccctgatccccggctccagcaccaccagcaccggcccctgccgcacctgcatgaccaccgcccagggcacctccatgtacccctcctgctgctgcaccaagcccagcgacggcaactgcacctgcatccccatccccagctcctgggccttcggcaagttcctgtgggagtgggccagcgcccgcttcagctggctgagcctgctggtgcccttcgtgcagtggttcgtgggcctgagccccaccgtgtggctgagcgtgatctggatgatgtggtactggggccccagcctgtacagcatcctgagccccttcctgcccctgctgcccatcttcttctgcctgtgggtgtacatcgactacaaggacgacgacgacaaggtcgaccaccaccaccaccaccactga

SEQ ID NO: 122 - protein, 242 aa, artificial sequence; identical to SEQ ID NO: 120
  synthetic polypeptide

SEQ ID NO: 123 - DNA, 5295 bp, artificial sequence
  synthetic oligonucleotide, pIRES-neo-2
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatctgcggcctagctagcgcttaaggcctgttaaccggtcgtacgtctccggattcgaattcggatccgcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 124 - DNA, 18 bp, artificial sequence
  synthetic oligonucleotide
caccaccaccaccaccac

SEQ ID NO: 125 - DNA, 18 bp, artificial sequence
  synthetic oligonucleotide
catcatcatcatcatcat

SEQ ID NO: 126 - DNA, 438 bp, artificial sequence
  synthetic oligonucleotide
atggactaccagggcatgctgcccgtgtgccccctgatccccggctccagcaccaccagcaccggcccctgccgcacctgcatgaccaccgcccagggcacctccatgtacccctcctgctgctgcaccaagcccagcgacggcaactgcacctgcatccccatcaccagctcctgggccttcggcaagttcctgtgggagtgggccagcgcccgcttcaactggctgagcctgctggtgcccttcgtgcagtggttcgtgggcctgagccccaccgtgtggctgagcgtgatctggatgatgtggtactggggccccagcctgtacagcatcctgaaccccttcctgcccctgctgcccatcttcttctgcctgtgggtgtacatcgactacaaggacgacgacgacaaggtcgaccaccaccaccaccaccactga

SEQ ID NO: 127 - protein, 145 aa, artificial sequence
  synthetic polypeptide
MDYQGMLPVCPLIPGSSTTSTGPCRTCMTTAQGTSMYPSCCCTKPSDGNCTCIPITSSWAFGKFLWEWASARFNWLSLLVPFVQWFVGLSPTVWLSVIWMMWYWGPSLYSILNPFLPLLPIFFCLWVYIDYKDDDDKVDHHHHHH

SEQ ID NO: 128 - DNA, 715 bp, artificial sequence
  synthetic oligonucleotide
ctcgaggattggggaccctgcgctgaacatggagaacatcacctccggcttcctgggccccctgctggtgctgcaggccggcttcttcctgctgacccgcatcctgaccatcccccagagcctggactcctggtggacctccctgaacttcctgggcggcaccaccgtgtgcctgggccagaactcccagtcccccacctccaaccactcccccacctcctgcccccccacctgccccggctaccgctggatgtgcctgcgccgcttcatcatcttcctgttcatcctgctgctgtgcctgatcttcctgctggtgctgctggactaccagggcatgctgcccgtgtgccccctgatccccggctccagcaccaccagcaccggcccctgccgcacctgcatgaccaccgcccagggcacctccatgtacccctcctgctgctgcaccaagcccagcgacggcaactgcacctgcatccccatccccagctcctgggccttcggcaagttcctgtgggagtgggccagcgcccgcttcagctggctgagcctgctggtgcccttcgtgcagtggttcgtgggcctgagccccaccgtgtggctgagcgtgatctggatgatgtggtactggggccccagcctgtacagcatcctgagccccttcctgcccctgctgcccatcttcttctgcctgtgggtgtacatcgatatctaa

SEQ ID NO: 129 - protein, 228 aa, artificial sequence
  synthetic polypeptide
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYIDI

(19) United States
(12) Patent Application Publication
(10) Pub. No.: US 2011/0052496 A1
(43) Pub. Date: Mar. 3, 2011

(54) HOLLOW NANOPARTICLES AND USES THEREOF

(76) Inventor: Angel Cid-Arregui, Heidelberg (DE)

(21) Appl. No.: 12/870,215

(22) Filed: Aug. 27, 2010

Related U.S. Application Data
(63) Continuation of application No. PCT/IB2009/005426, filed on Feb. 28, 2009.
(60) Provisional application No. 61/067,795, filed on Feb. 28, 2008.

(51) Int. Cl. A61K 49/00; A61K 9/14; A61K 39/395; A61K 39/29; C12N 5/10; C12N 1/00; C07K 2/00; C07H 21/04; C07K 14/02; A61P 35/00; A61P 1/16

(52) U.S. Cl. 424/9.1; 424/499; 424/130.1; 424/178.1; 424/192.1; 424/227.1; 435/325; 435/243; 530/324; 530/403; 536/23.72

(57) ABSTRACT

Aspects of the invention provide hollow nanoparticles and uses thereof. In particular, the invention provides membrane-enclosed vesicles comprising a truncated form of an HBsAg S protein lacking one or two of its amino-terminal transmembrane domains.

### RELATED APPLICATIONS

This application claims the benefit under 35 U.S.C. §119(e) from U.S. provisional application Ser. No. 61/067,795, filed Feb. 28, 2008, the entire contents of which are herein incorporated by reference.

### FIELD OF THE INVENTION

The invention relates to particles for delivering therapeutic or other agents.

### BACKGROUND OF THE INVENTION

Current treatment options for cancers, such as liver cancer (or hepatocellular carcinoma, HCC) include surgery, radiotherapy, chemotherapy and immunotherapy. In clinical practice, standard treatments typically include chemotherapeutic agents (cytotoxins or cytotoxic drugs) that inhibit cellular division. Despite many emerging therapeutic options, the efficacy of chemotherapy has not been surpassed by novel treatments for most cancers.

However, chemotherapeutic agents are far from ideal because they have been selected for their activity against proliferating cells, and they do not discriminate between tumor cells and normal cells undergoing rapid division. Consequently, chemotherapy often causes specific organ toxicities, and produces a series of adverse side effects, including hair and nail loss, mouth ulcers, and sickness. As a result, only 30%-50% of cancer patients are eligible for chemotherapy, depending on the tumor type, age, and physical conditions of the patient.

In addition, many potentially useful chemotherapeutic agents show poor water solubility and hence are difficult to deliver through traditional methods.

Attempts have been made to develop delivery systems to improve both targeting and efficacy of drug delivery. However, there are considerable problems with many of the current in vivo delivery systems for therapeutic drugs, making it difficult to treat many major illnesses and diseases.

### SUMMARY OF THE INVENTION

Aspects of the invention relate to novel methods and compositions for delivering therapeutic agents to patients. Aspects of the invention are based on novel particles that can encapsulate agents to be delivered to patients. The particles can increase delivery efficiency and/or targeting. In some embodiments, particles of the invention include a self-assembling protein domain that is optionally linked to one or more targeting domains. In certain embodiments, the self-assembling protein domain is derived from a modified form of a viral structural protein, for example, a modified form of the Hepatitis B Surface Antigen (HBsAg). Aspects of the invention are useful for delivering therapeutic agents for treating cancer. However, it should be appreciated that particles of the invention can be used to encapsulate and deliver any type of agent, including therapeutic agents for other diseases and/or diagnostic agents, for example.

Aspects of the present invention provide a therapeutic system based on nanoparticles suitable for encapsulation and delivery of a wide range of disease-treating substances to specific tissues and cells, wherein the nanoparticles comprise self-assembling protein domains that are optionally in the form of chimeric fusion proteins. In some embodiments, aspects of the invention relate to chimeric fusion proteins having the formula: N-terminus-AbCdE-C-terminus. In particular aspects of the invention, A and E are targeting domains, C is a domain favoring self assembly, and b and d are linker peptides. In particular aspects of the invention, the domain favoring self-assembly is of viral origin and the targeting domains are of either viral or non-viral origin. In particular aspects of the invention, the targeting domains of non-viral origin are of cellular origin, such as from a human or animal cell. In particular aspects of the invention, the linker may be of any natural or non-natural origin, including comprising synthetic de novo amino acids not found in nature. It should be appreciated that the chimeric fusion proteins may comprise fewer domains than AbCdE and any combination thereof, provided the chimeric fusion protein comprises at least the domain favoring self assembly (C). Examples for alternative chimeric fusion proteins are AbC, CdE, C, AC, CE, ACE, bCd, bC, or Cd. It also should be appreciated that in some embodiments one or more targeting domains may be included in the C domain (e.g., as an insertion into the C domain sequence or as a replacement of part of the C domain sequence). It should be appreciated that the internal targeting domain(s) may be linked via a synthetic linker at either or both of the N-terminal and C-terminal ends of the targeting domain(s). However, the internal targeting domains may be inserted without using a synthetic linker. It should be appreciated that a synthetic linker peptide as used herein is a peptide having a sequence that is different (or in a different location) from the natural sequence of A, C, or E, or a portion thereof.

In certain embodiments, a nanoparticle comprises a self-assembling protein domain (e.g., in the form of a self-assembling chimeric protein) of which the self-assembling moiety is of viral origin. In some embodiments, nanoparticles of the present invention comprise recombinant protein subunits derived from viral and/or cellular proteins or domains. It should be appreciated that nanoparticles of the invention that comprise recombinant protein subunits derived from viral proteins may be generally non-infectious and non-replicating and have no pathogenic potential. In certain embodiments, the recombinant protein subunits have self-assembly capacity and/or specific cell targeting properties. In certain embodiments, some recombinant protein subunits further facilitate penetration in the desired tissues and/or internalization in a target cell and/or intracellular release of the encapsulated agents or drugs.

Aspects of the invention provide nanoparticles that are adapted for delivering drugs (for example, cytotoxins or other drugs) to one or more target areas in a subject. In certain embodiments, the nanoparticles provided herein can be loaded with agents or drugs useful for therapeutic and/or diagnostic purposes. Nanoparticles may be loaded using any standard techniques such as electroporation or sonication in order to encapsulate the agents or drugs. Examples for loading water insoluble or partially soluble agents, for example Paclitaxel or docetaxel, are the use of solvents and freeze-drying, the fusion of nanoparticles to agents encapsulated into liposomes and the like. However, it should be appreciated that nanoparticles may be loaded using any suitable technique as the invention is not limited in this respect.

According to aspects of the invention, a nanoparticle can be administered to a patient via any suitable route. For example, nanoparticles may be administered via one or more routes including, but not limited to, subcutaneous, intramuscular, intravenous, and transdermal routes, and through mucosal layers such as via oral, and intranasal routes.

Aspects of the invention are based, at least in part, on the discovery that certain modified forms of Hepatitis B Surface Antigen (HBsAg) can form nanoparticles. According to aspects of the invention, these nanoparticles can be used for drug delivery. In some embodiments, nanoparticles of the invention have improved properties for drug loading and delivery. In view of the prior knowledge in the art, the nanoparticle-forming properties and the drug delivery properties of nanoparticles of the invention are unexpected. In certain embodiments, the viral polypeptide sequence is derived from the surface antigen of the hepatitis B virus lacking one or more N-terminal transmembrane domains (e.g., a modified form of HBsAg comprising the amino acid sequence any of SEQ ID NOs: 1-17, 57, 58, 60, 63, 67-69, 73-84). Accordingly, non-limiting examples of such modified forms of HBsAg are HBsAgΔ⁹⁸ (having an amino-terminal deletion of the first 98 amino acids of the HBsAg(S) protein of 226 amino acids), see FIG. 3A, or HBsAgΔ¹⁵³ (having an amino-terminal deletion of the first 153 amino acids of the HBsAg(S) protein of 226 amino acids), see FIG. 3A. In some aspects, such modified forms of HBsAg retain the ability to self-assemble and to form nanoparticles. In some aspects, such modified forms of HBsAg may be expressed in and purified from yeast (for example, P. pastoris). In some aspects, such modified forms of HBsAg may form nanoparticles within a size range between, for example, 20 and 30 nm.

According to aspects of the invention, nanoparticles can be formed using HBsAg protein variants that lack (for example, due to deletion and/or amino acid substitution) one or more N-terminal transmembrane domains of the S domain or chimeric fusion proteins thereof. Nanoparticles may be formed using variants of the S domain alone or in combination (for example, as a fusion protein or a mixture of proteins) with other HBsAg domains or other peptides or proteins. In certain embodiments, the chimeric fusion proteins include different moieties derived from various biological molecules (see, for example, FIGS. 2A-C).

Accordingly, aspects of the invention relate to nanoparticles having sizes that promote efficient delivery to target tissues. In some embodiments, nanoparticles based on modified HBsAg proteins of the invention are smaller than naturally-occurring hepatitis B viral particles and also smaller than particles based on HBsAg proteins that include the N-terminal transmembrane domains of the HBsAg S domain. In certain embodiments, HBsAg nanoparticles of the invention are smaller than 80 nm in diameter. Some HBsAg nanoparticles are smaller than 50 nm in diameter. Some HBsAg nanoparticles range between 10 and 40 nm in diameter. Some HBsAg nanoparticles range between 15 and 30 nm, and some may be about 20 nm to 30 nm in diameter. Certain nanoparticles of the invention are sufficiently small (for example, approximately 20 nm to 30 nm in diameter) to promote highly efficient cell entry (see, for example, Gao H. et al., PNAS 102(27):9469-74, 2005). However, it should be appreciated that larger nanoparticles of the invention (e.g., between 30 nm and 80 nm in diameter as described herein, or larger) also may be used to deliver drugs or other agents to target cells or tissues as described herein, as aspects of the invention are not limited in this respect.

Certain nanoparticles of the invention have a reduced density to optimize delivery capabilities. Without wishing to be bound by theory, some nanoparticles are expected to have less structural rigidity than their natural counterparts, thereby facilitating substance loading and/or delivery. For example, structural rigidity might be lowered in some nanoparticles as a result of fewer intra- and/or inter-molecular interactions due to the full or partial deletion and or substitution of one or more transmembrane domains of the HBsAg protein S domain. Without wishing to be bound by theory, this may lead to more flexible HBsAg S domains incorporated into the nanoparticle and/or to incorporation of more additional membrane material (such as for example lipids) into the nanoparticle. According to aspects of the invention, increased flexibility allows for improved drug and/or agent loading and/or delivery properties of nanoparticles of the invention. However, nanoparticles of the invention may retain a uniform structure that is useful for nanoscale fabrication and industrial scalability.

Nanoparticles of the invention may be loaded with (and capable of delivering) one or more cytotoxic drugs (for example, XELODA/Capecitabine, GEMZAR/Gemcitabine, TAXOTERE/Docetaxel, CAMPTO/Irinotecan, TAXOL/Paclitaxel) or other substances. In some embodiments, nanoparticles of the invention are engineered to evade the immune system of a host (for example, a human subject, for example a subject that has been vaccinated against Hepatitis B) by removing (by deletion and/or substitution) antigenic amino acids or sequences in a membrane protein of the nanoparticle (for example, in the ‘a’ determinant region of the HBsAg protein). In certain embodiments, the non-cellular or viral sequence within the fusion protein is designed to be as small as possible to limit immune responses by the subject.

In certain embodiments, nanoparticles of the invention may be engineered to display targeting molecules in a precise and reproducible spatial distribution at a nanoscale level (for example, as fusion proteins with the modified HBsAg proteins, or conjugated to the nanoparticle surface). Targeting molecules may be antibodies, ligands, receptors, or other molecules that can concentrate the nanoparticles at a target site (for example, by binding to a molecule that is preferentially expressed at the target site, for example, a diseased tissue such as a tumor or other cancer, or other diseased tissue or body target site of interest).

Nanoparticles of the invention also may be labeled with one or more agents that can be detected (for example, using a suitable imaging technology). These different embodiments may be used alone or combined with each other (for example, to produce a particle that is loaded with a therapeutic agent and also labeled with an imaging agent).

Accordingly, the invention provides technology that can be used to increase the efficacy and/or efficacy of drug delivery and/or allow for simultaneous diagnosis, treatment (for example, targeted treatment), and/or monitoring. However, other applications involve only drug delivery or diagnosis or monitoring.

In certain embodiments, the invention provides isolated hollow nanoparticles, which can be used for substance delivery, comprising a Hepatitis B Surface Antigen S protein domain (HBsAg S domain) having a truncation, the truncation comprising an amino-terminal deletion of at least one transmembrane domain, or a chimeric fusion protein thereof. In some embodiments, both of the amino-terminal transmembrane domains are lacking (for example, via deletion or mutation). In some embodiments nanoparticles comprising Hepatitis B Surface Antigen, HBsAg truncations or chimeric fusion proteins thereof (HBsAg nanoparticles) are used as a therapeutic in cancer therapy, delivering, for example cytotoxic drugs, or for DNA-based therapy (delivering genes), and can also be used to deliver vaccines and therapeutic antibodies, or siRNA and antisense mRNA, but they can also be used without an additional substance. In some embodiments, the invention provides pharmaceutical compositions comprising the HBsAg nanoparticles and a pharmaceutically acceptable carrier.

In certain embodiments, the invention provides methods for treating a subject having an adverse condition, comprising administering to the subject one or more compositions of the invention in an amount effective to treat the condition. In some embodiments, conditions that can be treated using the HBsAg nanoparticles of the invention are, for example, cancer (for example, hepatocellular carcinoma or HCC, bladder cancer, melanoma, pancreatic cancer, breast cancer, or other cancer), asthma, liver disease, heart disease, Alzheimer's disease, and age-related macular degeneration. However, embodiments of the invention may be used to deliver therapeutic agents to treat any diseased cell, tissue, or subject, as aspects of the invention are not limited in this respect.

In some embodiments, a nanoparticle of the invention (for example, lacking one or two transmembrane domains and/or an ‘a’ region of the HBsAg protein) may be used in a topical administration (for example, for bladder cancer or melanoma). In some embodiments, a nanoparticle of the invention (for example, lacking one or two transmembrane domains and/or an ‘a’ region of the HBsAg protein) may be used with a targeting molecule (for example, as a fusion protein) for intravenous (iv) administration (for example, to treat liver cancer, pancreatic cancer, breast cancer, or other organ cancer). In some embodiments, a nanoparticle of the invention (for example, lacking one or two transmembrane domains and/or an ‘a’ region of the HBsAg protein) may be used with a targeting molecule (for example, as a fusion protein) for oral administration.

In some embodiments HBsAg nanoparticles may be used to target a diagnostic, prophylactic or therapeutic substance to an adversely affected area in a subject, such as, for example a tumor. Aspects of the invention provide useful advantages over traditional systemic delivery techniques. Systemic, e.g., non-targeted, in vivo administration of cytotoxic drugs can produce many adverse side-effects from exposure of non-target organs. In addition, very high loading doses or repeated administrations are required for systemic delivery techniques to maintain therapeutic concentrations of a drug in a target area. Furthermore, the effectiveness of potentially useful substances, such as, for example, siRNA, therapeutic antibodies, and recombinant proteins can be hindered by their sometimes short biological half-lives in vivo. Without an effective technique for sustained and/or targeted delivery, many of these molecules are short-lived upon administration to a subject, for example due to dilution by body fluids, dissemination to other tissues, or being rapidly metabolized. Nanoparticles of the invention, e.g., certain HBsAg nanoparticles described herein, may be used to overcome many of these problems.

Certain HBsAg nanoparticles are modified to possess a reduced density through deletions and/or mutations of the HBsAg(S) protein, as described herein, in order to optimize their loading and delivery capabilities, thereby increasing their efficacy as compared to nanoparticles that do not comprise the deletions or mutations described herein.

Some HBsAg nanoparticles described herein are bio-degradable through metabolic pathways. In certain embodiments, the HBsAg(S) protein can be modified to introduce one or more protease recognition sites (e.g., intercalated between any of the integrated moieties). In some embodiments, a protease recognition site can be a thrombin recognition site or a factor Xa recognition site. However, any suitable protease recognition site (e.g., for a protein that is present in vivo in a subject, for example, but not limited to, a serum protease) may be used as aspects of the invention are not limited in this respect.

In some embodiments, the HBsAg nanoparticles of the invention possess the ability to carry cytotoxic drugs and display targeting molecules in a precise and reproducible spatial distribution. The targeting molecules can, in some embodiments, be targeting peptides, antibodies, or membrane receptors, directing the HBsAg nanoparticles to specific areas of the body of a subject, such as, for example, a tumor, a site of inflammation, a site of wound healing, a site of soft tissue damage, site of bone or cartilage damage, immune cell regeneration, across the blood-brain barrier, or a site of fat cell deposition. For example, a peptide within the Pre-S1 region of the Hepatitis B virus may be used to target hepatoma cells. Peptides may be used to target other sites (for example, peptide targeting αvβ6 integrin). Antibodies (for example, single chain antibodies) may be used to target diseased tissues (for example, antibodies that bind to tumor associated antigens or membrane receptors, for example MUC-1, CEA, Asialoglycoprotein Receptor, etc.). It should be appreciated that membrane receptors also may be targeted using natural ligands or fragments thereof. Targeting specific areas of the body of a subject may reduce toxicity. It should be appreciated that targeting molecules can be cloned N-terminally, C-terminally, and/or internally within the modified HBsAg proteins of the invention. For example, targeting molecules, such as a single chain antibody recognizing a tumor antigen on the surface of cancer cells can be fused N-terminally, C-terminally, and/or internally to the modified HBsAg proteins of the invention.

In some embodiments targeting molecules can be conjugated (for example, covalently or non-covalently) to the surface of the nanoparticle (for example, to a protein, lipid, carbohydrate, or other component of the membrane). In some embodiments, targeting molecules can be assembled into the nanoparticles (e.g., as part of a lipid membrane) without being conjugated or fused to any other components of the nanoparticle (e.g., without being conjugated or fused to an HBsAg protein of the invention.

In some embodiments, HBsAg nanoparticles may be modified to be detected by Magnetic Resonance Imaging (MRI). MRI may be used to provide an immediate monitoring of the therapeutic efficacy. MRI may allow simultaneous diagnosis and treatment, and may allow, for example, the diagnosis of early metastasis, or other adverse conditions. Agents that may be useful for detection by MRI, and which may be loaded into the nanoparticle and/or may be attached thereto, include, but are not limited to, 1) paramagnetic agents, such as Gadolinium-Diethylene triamine pentaacetic acid (Gd-DTPA), which may be coupled or bound to monoclonal antibodies; Ferrioxamine methanesulfonate ultra-small super-paramagnetic iron oxide (USPIO), which may be used for example for lymph node imaging; 2) non-ionic agents, such as Gadodiamide, Gadoteridol; 3) Gd-labeled albumin; 4) 51^{Cr}-labeled nanoparticles; 5) Metalloporphyrins, such as Mn(III) TPPS4 (manganese(III) tetra-[4-sulfanatophenyl]porphyrin); and/or 6) Nitroxides. In certain embodiments, these agents are encapsulated by nanoparticles. For example, both Gd-DTPA and MnCl₂ can be encapsulated into the aqueous inner chamber of liposomes or nanoparticles. Encapsulation of super-paramagnetic iron oxide particles into liposomes, for example, results in nanoparticles referred to in the literature as “ferrosomes.” Non-limiting examples of useful agents are described, for example, in Magnetic Resonance Imaging by Stark and Bradley, second edition, C.V. Mosby Co., 1988; Clinical Magnetic Resonance Imaging and Spectroscopy by Andrew et al. Wiley, 1990; The Essential Physics of Medical Imaging by Bushberg et al. second edition, Lippincott Williams & Wilkins, 2002; Christensen's Physics of Diagnostic Radiology, fourth edition, Lippincott Williams & Wilkins, 1990; Abdominal Magnetic Resonance Imaging by Ros and Bidgood, Harcourt Health Sciences, 1993; Fast-Scan Magnetic Resonance Principles and Applications by Felix Wehrli; Dynamic magnetic resonance imaging of human brain activity during primary sensory stimulation by Kwong et al. Proc. Natl. Acad. Sci. USA, June 1992.

In certain embodiments, the DNA sequence encoding the HBsAg chimeric fusion protein is optimized for expression in yeast cells by generating a synthetic gene carrying a nucleotide sequence entirely made of codons preferred by highly expressed yeast genes. In certain embodiments, the DNA sequence encoding the chimeric fusion protein is optimized for expression in mammalian cells, and particularly in human cells, by generating a synthetic gene carrying a nucleotide sequence entirely made of codons preferred by highly expressed mammalian genes (e.g., human genes).

In certain embodiments, yeast or human codon optimized genes have the nucleotide sequence of one of SEQ ID NOs: 18-51 and 85-118.

In some embodiments, the invention provides isolated nucleic acids (e.g., DNAs) encoding a Hepatitis B Surface Antigen S domain protein having a truncation, the truncation comprising an amino-terminal deletion of at least one transmembrane domain. In some embodiments, the invention provides isolated Hepatitis B Surface Antigen S domain proteins having a truncation, the truncation comprising an amino-terminal deletion of at least one transmembrane domain.

In some embodiments, the invention provides isolated DNAs encoding a chimeric fusion protein comprising a modified Hepatitis B Surface Antigen S domain protein having a truncation, the truncation comprising an amino-terminal deletion of at least one transmembrane domain, and which is fused amino-terminal or carboxy-terminal or has inserted within the Hepatitis B Surface Antigen S domain sequence one or more targeting peptides or domains (for example integrin, pre-S1) or other peptides or domains that add functionality (for example albumin, purification tags). Provided herein are also the corresponding isolated proteins.

In some embodiments, the invention provides isolated DNAs encoding a chimeric fusion protein or a truncated Hepatitis B Surface Antigen S domain protein described herein further comprising additional deletions and/or nucleotides substitutions to alter the amino acid sequence such that the chimeric fusion protein or a truncated Hepatitis B Surface Antigen S domain protein is less immunogenic to a subject. Provided herein are also the corresponding isolated proteins.

In some embodiments, the invention provides isolated DNAs encoding a chimeric fusion protein or a truncated Hepatitis B Surface Antigen S domain protein described herein further comprising nucleotide sequences that are codon optimized for increased expression in a particular organism (for example yeast, mammalian cells).

The invention also relates to a method of making a medicament for use in treating one or more diseases or conditions in a subject (e.g., a human or other mammalian subject). Such medicaments can be used for prophylactic treatment of a subject at risk for or suspected of having a disease or condition. Accordingly, one or more recombinant or fusion proteins or related nanoparticles described herein may be used for the preparation of a medicament for use in any of the methods of treatment described herein. In some embodiments, the invention provides for the use of one or more proteins or compositions of the invention for the manufacture of a medicament or pharmaceutical for treating a mammal (e.g., a human) having one or more symptoms of, or at risk for, one or more diseases or conditions (e.g., cancer). Accordingly, aspects of the invention relate to the use of one or more proteins or compositions of the invention for the preparation of a medicament for treating or preventing a disease or condition (e.g., cancer) in a subject.

Accordingly, the invention also relates to one or more compounds or compositions of the invention for use as a medicament. The invention also relates to one or more of these compounds or compositions for use in methods of the invention.

### BRIEF DESCRIPTION OF THE DRAWINGS

FIG. 1 illustrates Hepatitis B surface protein genes. FIG. 1A shows the organization of the HBsAg protein-encoding genes, FIG. 1B shows a schematic depiction of the HBsAg(S) protein, and FIG. 1C is a schematic representation of an HBsAg particle.

FIG. 2 depicts a schematic representation of non-limiting examples of HBsAg nanoparticles of the invention. In FIG. 2A, a nanoparticle includes a chimeric fusion protein having a truncated Hepatitis B Surface Antigen S domain protein fused N-terminally to a targeting peptide and C-terminally to an albumin binding sequence. In FIG. 2B, a nanoparticle includes a chimeric fusion protein with different truncated Hepatitis B Surface Antigen S domain proteins fused to different additional functional peptides or protein domains, such as either N-terminally or C-terminally to either a targeting peptide or to an albumin binding sequence. In FIG. 2C, a nanoparticle includes a chimeric fusion protein having different truncated Hepatitis B Surface Antigen S domain proteins wherein one S domain protein is fused to additional functional peptides or protein domains and one is not.

FIG. 3 illustrates non-limiting examples of truncated HBsAg(S) proteins and nanoparticles containing them. FIG. 3A shows a schematic of HBsAgΔ⁹⁸ and HBsAgΔ¹⁵³. FIG. 3B shows an example of a scanning electron microscope image of nanoparticles containing HBsAgΔ⁹⁸.

FIG. 4 shows DNA and amino acid sequences of non-limiting examples of modified HBsAg(S) proteins. FIG. 4A shows a full-length HBsAg(S) protein codon-optimized for yeast. FIG. 4B shows an HBsAg lacking domains I and II.

FIG. 5 shows a Western blot non-limiting examples of truncated forms of an HBsAg protein.

FIG. 6 shows a Western blot non-limiting examples of truncated forms of an HBsAg protein.

FIG. 7 depicts a bar graph showing a cell viability assay in vitro for cells treated with non-limiting embodiments of nanoparticles of the invention.

### DESCRIPTION OF SEQUENCES

The following sequence descriptions represent non-limiting embodiments that illustrate aspects of the invention. Specific sequences are described in more detail herein, including in the tables, and in the attached Sequence Listing. The following sequence descriptions illustrate examples of a chimeric protein of the general formula: N-terminus-AbCdE-C-terminus, wherein A and E are targeting domains, C is a self-assembling domain, and b and d are linker peptides. These non-limiting sequence descriptions illustrate embodiments that all include a C domain. In some embodiments, the C domain includes a targeting motif itself (within the C domain sequence). In some embodiments, a targeting motif is linked to the N-terminus of the C domain. In some embodiments, a targeting motif is linked to the C-terminus of the C domain. Accordingly, in some embodiments of the invention a chimeric protein may include an N-terminal targeting domain, an internal targeting domain, a C-terminal targeting domain, or any combination of two or more thereof. It should be appreciated that the targeting domains may be attached to the C domain via one or more synthetic linker sequences (e.g., at the C-terminal end of an N-terminal targeting domain, at the N-terminal end of a C-terminal targeting domain, or at both the N-terminus and C-terminus of an internal targeting domain).

It should be appreciated that any amino acid sequence, such as any natural or synthetic peptide or protein domain, may be attached to the C domain with or without linker sequences. These include peptides or a protein domains that provide functions other than targeting functions. It also should be appreciated that chimeric proteins of the invention may include one or more purification and or detection tags (e.g., at the N-terminus, at the C-terminus, internally, or any combination thereof). Some of the sequences provided herein illustrate non-limiting examples of chimeric proteins containing one or more purification and/or detection tags. It should be appreciated that proteins of the invention may have one or more deletions and/or point mutations relative to the wild-type protein sequences of some of the domains, as described herein, and as illustrated by some of the non-limiting sequences provided as examples.

SEQ ID NO:1 is the amino acid sequence of chimeric protein HBsΔ⁹⁸. The initial methionine at position 98 was introduced to replace Leu at this position in the original sequence and immune-escape mutations within the “a” determinant (Thr140Ser, Lys and Gly145Arg) were introduced. SEQ ID NO: 57 is the amino acid sequence of chimeric protein HBsΔ⁹⁸FH with a C-terminal detection tag, (Flag DYKDDDDK, SEQ ID NO: 52) and a purification tag (hexa-His tag HHHHHH, SEQ ID NO: 53). It should be appreciated that the history can be encoded by CACo and/or CAu codons (see, SEQ ID NOs: 124 and 125, respectively). The initial methionine at position 98 was introduced to replace Leu at this position in the original sequence and immune-escape mutations within the “a” determinant (Thr140Ser, Lys141Glu and Gly145Arg) were introduced. SEQ ID NO: 60 (NIgKHBsΔ⁹⁸FH) is the amino acid sequence of the chimeric protein of SEQ ID NO: 57 with an additional N-terminal IgK signal peptide. It should be appreciated that this signal peptide may be added with or without a linker peptide and with or without a detection and/or purification tag.

SEQ ID NO: 2 is the amino acid sequence of chimeric protein HBsΔ¹⁵³. The initial methionine at position 153 was added upstream of the Ser at position 154 in the original sequence and immune-escape mutations within the “a” determinant (Thr140Ser, Lys141Glu and Gly145Arg) were introduced. SEQ ID NO: 58 is the amino acid sequence of chimeric protein HBsΔ¹⁵³FH with a C-terminal Flag and hexa-His tags. The initial methionine at position 153 was added upstream of the Ser at position 154 in the original sequence and immune-escape mutations within the “a” determinant (Thr140Ser, Lys141Glu and Gly145Arg) were introduced. SEQ ID NO: 63 (NIgKHBsΔ¹⁵³FH) is the amino acid sequence of the chimeric protein of SEQ ID NO: 58 with an additional N-terminal IgK signal peptide. It should be appreciated that this signal peptide may be added with or without a linker peptide and with or without a detection and/or purification tag.

SEQ ID NO: 3 is the amino acid sequence of chimeric protein NHep1HBsΔ⁹⁸ comprising the sequence: PLGFFPDHQLDPAFGANSNNPDWDFNP (SEQ ID NO: 54), which comprises a domain of the preS1 involved in hepatitis B virus attachment to cell membrane receptors. This domain is used as a targeting domain and may be linked to the N-terminus of the polypeptide HBsΔ⁹⁸ via a synthetic linker peptide (for example 2×GGGGS, SEQ ID NO: 55). SEQ ID NO: 67 (NIgKHep1Cabd HBsΔ⁹⁸) is the amino acid sequence of the chimeric protein of SEQ ID NO: 3 with an additional N-terminal IgK signal peptide and a C-terminal albumin binding domain.

SEQ ID NO: 4 is the amino acid sequence of chimeric protein CHep1HBsΔ⁹⁸ comprising the sequence of SEQ ID NO: 54, which comprises a “targeting” domain of the preS1 involved in hepatitis B virus attachment to cell membrane receptors, which may be linked to the C-terminus of the polypeptide HBsΔ⁹⁸ via a synthetic linker peptide (for example 2×GGGGS, SEQ ID NO: 55). SEQ ID NO: 68 (NIgKabdCHep1HBsΔ⁹⁸) is the amino acid sequence of the chimeric protein of SEQ ID NO: 4 with an additional N-terminal IgK signal peptide and an N-terminal albumin binding domain.

SEQ ID NO: 5 is the amino acid sequence of chimeric protein aHep1HBsΔ⁹⁸ comprising the sequence of SEQ ID NO: 54, which comprises a “targeting” domain of the preS1 involved in hepatitis B virus attachment to cell membrane receptors which has been inserted replacing the second loop of the “a” determinant. This sequence may be linked via N-terminal and C-terminal synthetic linker peptides (SEQ ID NO: 55). SEQ ID NO: 69 (NIgKaHep1CabdHBsΔ⁹⁸) is the amino acid sequence of the chimeric protein of SEQ ID NO: 5 with an additional N-terminal IgK signal peptide and a C-terminal albumin binding domain.

SEQ ID NO: 6 is the amino acid sequence of chimeric protein NaHep1HBsΔ⁹⁸ comprising two sequences of SEQ ID NO: 54, that comprise a “targeting” domain of the preS1 involved in hepatitis B virus attachment to cell membrane receptors. The two sequences are located both at the N-terminus and replacing the second loop of the “a” determinant. Each sequence may be connected via synthetic linker peptides (SEQ ID NO: 55). The N-terminal targeting domain may be connected via a C-terminal synthetic linker peptide. The internal targeting domain may be connected via both N-terminal and C-terminal synthetic linker peptides (SEQ ID NO: 55). SEQ ID NO: 73 (NIgKaHep1CabdHBsΔ⁹⁸) is the amino acid sequence of the chimeric protein of SEQ ID NO: 6 with an additional N-terminal IgK signal peptide and a C-terminal albumin binding domain.

SEQ ID NO: 7 is the amino acid sequence of chimeric protein aCHep1HBsΔ⁹⁸ comprising two sequences of SEQ ID NO: 54, that comprise a “targeting” domain of the preS1 involved in hepatitis B virus attachment to cell membrane receptors. The two sequences are both at the C-terminus and replacing the second loop of the “a” determinant. These peptides may be connected via synthetic linkers (for example, SEQ ID NO: 55). SEQ ID NO: 74 (NIgKabdaCHep1HBsΔ⁹⁸) is the amino acid sequence of the chimeric protein of SEQ ID NO: 7 with an additional N-terminal IgK signal peptide and an N-terminal albumin binding domain.

SEQ ID NO: 8 is the amino acid sequence of chimeric protein NHep1HBsΔ⁹⁸KDR. The sequence at the N-terminus SEQ ID NO: 54 comprises the preS1 “targeting” domain involved in hepatitis B virus attachment to cell membrane receptors. The sequence at the C-terminus: SGDSRVCWEDSWGGEVCFRYDP (SEQ ID NO: 59) comprises a sequence binding the vascular endothelial growth factor receptor. This also is provided as a targeting domain. Both sequences may be connected via synthetic linker peptides (for example, 2×GGGGS SEQ ID NO: 55 and/or AAAAS, SEQ ID NO: 56). SEQ ID NO: 75 (NIgKHep1CabdHBsΔ⁹⁸ KDR) is the amino acid sequence of the chimeric protein of SEQ ID NO: 8 with an additional N-terminal IgK signal peptide and a C-terminal albumin binding domain.

SEQ ID NO: 9 is the amino acid sequence of chimeric protein NHep1HBsΔ¹⁵³ comprising the sequence of SEQ ID NO: 54, which comprises a “targeting” domain of the preS1 involved in hepatitis B virus attachment to cell membrane receptors linked to the N-terminus of the polypeptide HBsΔ⁹⁸, for example, via a synthetic linker peptide (2×GGGGS, SEQ ID NO: 55). SEQ ID NO: 76 (NIgKHep1CabdHBsΔ¹⁵³) is the amino acid sequence of the chimeric protein of SEQ ID NO: 9 with an additional N-terminal IgK signal peptide and a C-terminal albumin binding domain.

SEQ ID NO: 10 is the amino acid sequence of chimeric protein CHep1HBsΔ¹⁵³ comprising the sequence of SEQ ID NO: 54, which comprises a “targeting” domain of the preS1 involved in hepatitis B virus attachment to cell membrane receptors linked to the C-terminus of the polypeptide HBsΔ¹⁵³, for example, via a synthetic linker peptide (2×GGGGS, SEQ ID NO: 55). SEQ ID NO: 77 (NIgKabdCHep1HBsΔ¹⁵³) is the amino acid sequence of the chimeric protein of SEQ ID NO: 10 with an additional N-terminal IgK signal peptide and an N-terminal albumin binding domain.

SEQ ID NO: 11 is the amino acid sequence of chimeric protein NCHep1HBsΔ¹⁵³ comprising two sequences of SEQ ID NO: 54, that comprise a “targeting” domain of the preS1 involved in hepatitis B virus attachment to cell membrane receptors and may be linked both at the N- and the C-termini via synthetic linker peptides (for example SEQ ID NO: 55). SEQ ID NO: 78 (NIgkNCHep1HBsΔ¹⁵³) is the amino acid sequence of the chimeric protein of SEQ ID NO: 11 with an additional N-terminal IgK signal peptide.

SEQ ID NO: 12 is the amino acid sequence of chimeric protein NaL1HBsΔ¹⁵³. This protein contains domains of the L1 protein of HPV-16, L1⁸¹⁻¹⁰⁰: PNNNKILVPKVSGLQYRVFR (SEQ ID NO: 61) and L1³⁰¹⁻³²⁰: LYIKGSGSTANLASSNYFPT (SEQ ID NO: 62), which neutralize uptake of HPV16 and HPV18 VLPs, at the N-terminus and within the HBsΔ⁹⁸ sequence replacing the second loop of the “a” determinant. The “targeting” domains may be linked to the modified HBsAg domain via linker peptides (for example SEQ ID NO: 55). SEQ ID NO: 79 (NIgKaL1CabdHBsΔ¹⁵³) is the amino acid sequence of the chimeric protein of SEQ ID NO: 12 with an additional N-terminal IgK signal peptide and a C-terminal albumin binding domain.

SEQ ID NO: 13 is the amino acid sequence of chimeric protein NCL1HBsΔ¹⁵³. This protein contains domains of the L1 protein of HPV-16, L1⁸¹⁻¹⁰⁰(SEQ ID NO: 61) and L1³⁰¹⁻³²⁰ (SEQ ID NO: 62) flanking the HBsΔ¹⁵³ polypeptide. The “targeting” domains may be linked to the modified HBsAg domain via linker peptides (for example SEQ ID NO: 55). SEQ ID NO: 80 (NIgKNCL1HBsΔ¹⁵³) is the amino acid sequence of the chimeric protein of SEQ ID NO: 13 with an additional N-terminal IgK signal peptide.

SEQ ID NO: 14 is the amino acid sequence of chimeric protein NaIBPHBsΔ⁹⁸. This protein contains two integrin binding domains: GRGDSP (SEQ ID NO: 64) and PHSRN (SEQ ID NO: 65) derived from fibronectin. One or more N- and/or C-terminal linker linker peptides (for example SEQ ID NO: 55) may be used to link or integrate these domains to the modified HBsAg domain. SEQ ID NO: 81 (NIgKaIBPCabdHBsΔ⁹⁸) is the amino acid sequence of the chimeric protein of SEQ ID NO: 14 with an additional N-terminal IgK signal peptide and a C-terminal albumin binding domain.

SEQ ID NO: 15 is the amino acid sequence of chimeric protein NCIBPHBsΔ¹⁵³. This protein contains two integrin-binding domains SEQ ID NO: 64 and SEQ ID NO: 65 derived from fibronectin. One or more N- and/or C-terminal linker linker peptides (for example SEQ ID NO: 55) may be used to link or integrate these domains to the modified HBsAg domain. SEQ ID NO: 82 (NIgKabdCIBPHBsΔ¹⁵³) is the amino acid sequence of the chimeric protein of SEQ ID NO: 15 with an additional N-terminal IgK signal peptide and an N-terminal albumin binding domain.

SEQ ID NO: 16 is the amino acid sequence of chimeric protein NaFRBHBsΔ⁹⁸. This protein contains three copies of a fibroblast growth factor receptor-binding domain: MQLPLAT (SEQ ID NO: 66). One or more N- and/or C-terminal linker linker peptides (for example SEQ ID NO: 55) may be used to link or integrate these domains to the modified HBsAg domain. SEQ ID NO: 83 (NIgKaFRBHBsΔ⁹⁸) is the amino acid sequence of the chimeric protein of SEQ ID NO: 16 with an additional N-terminal IgK signal peptide.

SEQ ID NO: 17 is the amino acid sequence of chimeric protein NCFRBHBsΔ¹⁵³. This protein contains three copies of a fibroblast growth factor receptor-binding domain (SEQ ID NO: 66). One or more N- and/or C-terminal linker linker peptides (for example SEQ ID NO: 55) may be used to link or integrate these domains to the modified HBsAg domain. SEQ ID NO: 84 (NIgKNCFRBHBsΔ¹⁵³) is the amino acid sequence of the chimeric protein of SEQ ID NO: 17 with an additional N-terminal IgK signal peptide.

It should be appreciated that any of the signal peptides, albumin binding domains, detection, and/or purification tags illustrated or described herein, or equivalents thereof, may be fused alone, or in combination, at the N-terminus, C-terminus, and/or internally, with any of the chimeric proteins described herein, with or without one or more linker peptides, optionally including additional amino acid changes (e.g., to remove one or more antigenic sequences).

The following sequences (SEQ ID NOs: 18-51 and 85-118) provide non-limiting examples of codon-optimized nucleic acid sequences encoding non-limiting examples of chimeric proteins of the invention.

SEQ ID NO: 18 is the nucleotide sequence of a synthetic gene encoding HBsΔ⁹⁸FH (SEQ ID NO: 57) with codon usage optimized for expression in yeast.

SEQ ID NO: 19 is the nucleotide sequence of a synthetic gene encoding HBsΔ⁹⁸FH (SEQ ID NO: 57) with codon usage optimized for expression in human cells.

SEQ ID NO: 20 is the nucleotide sequence of a synthetic gene encoding HBsΔ¹⁵³FH (SEQ ID NO: 58) with codon usage optimized for expression in yeast.

SEQ ID NO: 21 is the nucleotide sequence of a synthetic gene encoding HBsΔ¹⁵³FH (SEQ ID NO: 58) with codon usage optimized for expression in human cells.

SEQ ID NO: 22 is the nucleotide sequence of a synthetic gene encoding NHep1HBsΔ⁹⁸ (SEQ ID NO: 3) with codon usage optimized for expression in yeast.

SEQ ID NO: 23 is the nucleotide sequence of a synthetic gene encoding NHep1HBsΔ⁹⁸ (SEQ ID NO: 3) with codon usage optimized for expression in human cells.

SEQ ID NO: 24 is the nucleotide sequence of a synthetic gene encoding CHep1HBsΔ⁹⁸ (SEQ ID NO: 4) with codon usage optimized for expression in yeast.

SEQ ID NO: 25 is the nucleotide sequence of a synthetic gene encoding CHep1HBsΔ⁹⁸ (SEQ ID NO: 4) with codon usage optimized for expression in human cells.

SEQ ID NO: 26 is the nucleotide sequence of a synthetic gene encoding aHep1HBsΔ⁹⁸ (SEQ ID NO: 5) with codon usage optimized for expression in yeast.

SEQ ID NO: 27 is the nucleotide sequence of a synthetic gene encoding aHep1HBsΔ⁹⁸ (SEQ ID NO: 5) with codon usage optimized for expression in human cells.

SEQ ID NO: 28 is the nucleotide sequence of a synthetic gene encoding NaHep1HBsΔ⁹⁸ (SEQ ID NO: 6) with codon usage optimized for expression in yeast.

SEQ ID NO: 29 is the nucleotide sequence of a synthetic gene encoding NaHep1HBsΔ⁹⁸ (SEQ ID NO: 6) with codon usage optimized for expression in human cells.

SEQ ID NO: 30 is the nucleotide sequence of a synthetic gene encoding aCHep1HBsΔ⁹⁸ (SEQ ID NO: 7) with codon usage optimized for expression in yeast.

SEQ ID NO: 31 is the nucleotide sequence of a synthetic gene encoding aHep1HBsΔ⁹⁸ (SEQ ID NO: 7) with codon usage optimized for expression in human cells.

SEQ ID NO: 32 is the nucleotide sequence of a synthetic gene encoding NHep1HBsΔ⁹⁸ KDR (SEQ ID NO: 8) with codon usage optimized for expression in yeast.

SEQ ID NO: 33 is the nucleotide sequence of a synthetic gene encoding NHep1HBsΔ⁹⁸ KDR (SEQ ID NO: 8) with codon usage optimized for expression in human cells.

SEQ ID NO: 34 is the nucleotide sequence of a synthetic gene encoding NHep1HBsΔ¹⁵³ (SEQ ID NO: 9) with codon usage optimized for expression in yeast.

SEQ ID NO: 35 is the nucleotide sequence of a synthetic gene encoding NHep1HBsΔ¹⁵³ (SEQ ID NO: 9) with codon usage optimized for expression in human cells.

SEQ ID NO: 36 is the nucleotide sequence of a synthetic gene encoding CHep1HBsΔ¹⁵³ (SEQ ID NO: 10) with codon usage optimized for expression in yeast.

SEQ ID NO: 37 is the nucleotide sequence of a synthetic gene encoding CHep1HBsΔ¹⁵³ (SEQ ID NO: 10) with codon usage optimized for expression in human cells.

SEQ ID NO: 38 is the nucleotide sequence of a synthetic gene encoding NCHep1HBsΔ¹⁵³ (SEQ ID NO: 11) with codon usage optimized for expression in yeast.

SEQ ID NO: 39 is the nucleotide sequence of a synthetic gene encoding NCHep1HBsΔ¹⁵³ (SEQ ID NO: 11) with codon usage optimized for expression in human cells.

SEQ ID NO: 40 is the nucleotide sequence of a synthetic gene encoding NaL1HBsΔ¹⁵³ (SEQ ID NO: 12) with codon usage optimized for expression in yeast.

SEQ ID NO: 41 is the nucleotide sequence of a synthetic gene encoding NaL1HBsΔ¹⁵³ (SEQ ID NO: 12) with codon usage optimized for expression in human cells.

SEQ ID NO: 42 is the nucleotide sequence of a synthetic gene encoding NCL1HBsΔ¹⁵³ (SEQ ID NO: 13) with codon usage optimized for expression in yeast.

SEQ ID NO: 43 is the nucleotide sequence of a synthetic gene encoding NCL1HBsΔ¹⁵³ (SEQ ID NO: 13) with codon usage optimized for expression in human cells.

SEQ ID NO: 44 is the nucleotide sequence of a synthetic gene encoding NaIBPHBsΔ⁹⁸ (SEQ ID NO: 14) with codon usage optimized for expression in yeast.

SEQ ID NO: 45 is the nucleotide sequence of a synthetic gene encoding NaIBPHBsΔ⁹⁸ (SEQ ID NO: 14) with codon usage optimized for expression in human cells.

SEQ ID NO: 46 is the nucleotide sequence of a synthetic gene encoding NCIBPHBsΔ¹⁵³ (SEQ ID NO: 15) with codon usage optimized for expression in yeast.

SEQ ID NO: 47 is the nucleotide sequence of a synthetic gene encoding NCIBPHBsΔ¹⁵³ (SEQ ID NO: 15) with codon usage optimized for expression in human cells.

SEQ ID NO: 48 is the nucleotide sequence of a synthetic gene encoding NaFRBHBsΔ⁹⁸ (SEQ ID NO: 16) with codon usage optimized for expression in yeast.

SEQ ID NO: 49 is the nucleotide sequence of a synthetic gene encoding NaFRBHBsΔ⁹⁸ (SEQ ID NO: 16) with codon usage optimized for expression in human cells.

SEQ ID NO: 50 is the nucleotide sequence of a synthetic gene encoding NCFRBHBsΔ¹⁵³ (SEQ ID NO: 17) with codon usage optimized for expression in yeast.

SEQ ID NO: 51 is the nucleotide sequence of a synthetic gene encoding NCFRBHBsΔ¹⁵³ (SEQ ID NO: 17) with codon usage optimized for expression in human cells.

SEQ ID NO: 85 is the nucleotide sequence of a synthetic gene encoding NIgKHBsΔ⁹⁸FH (SEQ ID NO: 60) with codon usage optimized for expression in yeast.

SEQ ID NO: 86 is the nucleotide sequence of a synthetic gene encoding NIgKHBsΔ⁹⁸FH (SEQ ID NO: 60) with codon usage optimized for expression in human cells.

SEQ ID NO: 87 is the nucleotide sequence of a synthetic gene encoding NIgKHBsΔ¹⁵³FH (SEQ ID NO: 63) with codon usage optimized for expression in yeast.

SEQ ID NO: 88 is the nucleotide sequence of a synthetic gene encoding NIgKHBsΔ¹⁵³FH (SEQ ID NO: 63) with codon usage optimized for expression in human cells.

SEQ ID NO: 89 is the nucleotide sequence of a synthetic gene encoding NIgKHep1CabdHBsΔ⁹⁸ (SEQ ID NO: 67) with codon usage optimized for expression in yeast.

SEQ ID NO: 90 is the nucleotide sequence of a synthetic gene encoding NIgKHep1CabdHBsΔ⁹⁸ (SEQ ID NO: 67) with codon usage optimized for expression in human cells.

SEQ ID NO: 91 is the nucleotide sequence of a synthetic gene encoding NIgKabdCHep1HBsΔ⁹⁸ (SEQ ID NO: 68) with codon usage optimized for expression in yeast.

SEQ ID NO: 92 is the nucleotide sequence of a synthetic gene encoding NIgKabdCHep1HBsΔ⁹⁸ (SEQ ID NO: 68) with codon usage optimized for expression in human cells.

SEQ ID NO: 93 is the nucleotide sequence of a synthetic gene encoding NIgKaHep1Cabd HBsΔ⁹⁸ (SEQ ID NO: 69) with codon usage optimized for expression in yeast.

SEQ ID NO: 94 is the nucleotide sequence of a synthetic gene encoding NIgKaHep1CabdHBsΔ⁹⁸ (SEQ ID NO: 69) with codon usage optimized for expression in human cells.

SEQ ID NO: 95 is the nucleotide sequence of a synthetic gene encoding NIgKaHep1CabdHBsΔ⁹⁸ (SEQ ID NO: 73) with codon usage optimized for expression in yeast.

SEQ ID NO: 96 is the nucleotide sequence of a synthetic gene encoding NIgKaHep1Cabd HBsΔ⁹⁸ (SEQ ID NO: 73) with codon usage optimized for expression in human cells.

SEQ ID NO: 97 is the nucleotide sequence of a synthetic gene encoding NIgKabdaCHep1HBsΔ⁹⁸ (SEQ ID NO: 74) with codon usage optimized for expression in yeast.

SEQ ID NO: 98 is the nucleotide sequence of a synthetic gene encoding NIgKabdaCHep1HBsΔ⁹⁸ (SEQ ID NO: 74) with codon usage optimized for expression in human cells.

SEQ ID NO: 99 is the nucleotide sequence of a synthetic gene encoding NIgKHep1CabdHBsΔ⁹⁸ KDR (SEQ ID NO: 75) with codon usage optimized for expression in yeast.

SEQ ID NO: 100 is the nucleotide sequence of a synthetic gene encoding NIgKHep1CabdHBsΔ⁹⁸ KDR (SEQ ID NO: 75) with codon usage optimized for expression in human cells.

SEQ ID NO: 101 is the nucleotide sequence of a synthetic gene encoding NIgKHep1Cabd HBsΔ¹⁵³ (SEQ ID NO: 76) with codon usage optimized for expression in yeast.

SEQ ID NO: 102 is the nucleotide sequence of a synthetic gene encoding NIgKHep1CabdHBsΔ¹⁵³ (SEQ ID NO: 76) with codon usage optimized for expression in human cells.

SEQ ID NO: 103 is the nucleotide sequence of a synthetic gene encoding NIgKabdCHep1 HBsΔ¹⁵³ (SEQ ID NO: 77) with codon usage optimized for expression in yeast.

SEQ ID NO: 104 is the nucleotide sequence of a synthetic gene encoding NIgKabdCHep1HBsΔ¹⁵³ (SEQ ID NO: 77) with codon usage optimized for expression in human cells.

SEQ ID NO: 105 is the nucleotide sequence of a synthetic gene encoding NIgkNCHep1HBsΔ¹⁵³ (SEQ ID NO: 78) with codon usage optimized for expression in yeast.

SEQ ID NO: 106 is the nucleotide sequence of a synthetic gene encoding NIgkNCHep1HBsΔ¹⁵³ (SEQ ID NO: 78) with codon usage optimized for expression in human cells.

SEQ ID NO: 107 is the nucleotide sequence of a synthetic gene encoding NIgKaL1CabdHBsΔ¹⁵³ (SEQ ID NO: 79) with codon usage optimized for expression in yeast.

SEQ ID NO: 108 is the nucleotide sequence of a synthetic gene encoding NIgKaL1CabdHBsΔ¹⁵³ (SEQ ID NO: 79) with codon usage optimized for expression in human cells.

SEQ ID NO: 109 is the nucleotide sequence of a synthetic gene encoding NIgKNCL1HBsΔ¹⁵³ (SEQ ID NO: 80) with codon usage optimized for expression in yeast.

SEQ ID NO: 110 is the nucleotide sequence of a synthetic gene encoding NIgKNCL1HBsΔ¹⁵³ (SEQ ID NO: 80) with codon usage optimized for expression in human cells.

SEQ ID NO: 111 is the nucleotide sequence of a synthetic gene encoding NIgKaIBPCabdHBsΔ⁹⁸ (SEQ ID NO: 81) with codon usage optimized for expression in yeast.

SEQ ID NO: 112 is the nucleotide sequence of a synthetic gene encoding NIgKaIBPCabdHBsΔ⁹⁸ (SEQ ID NO: 81) with codon usage optimized for expression in human cells.

SEQ ID NO: 113 is the nucleotide sequence of a synthetic gene encoding NIgKabdCIBPHBsΔ¹⁵³ (SEQ ID NO: 82) with codon usage optimized for expression in yeast.

SEQ ID NO: 114 is the nucleotide sequence of a synthetic gene encoding NIgKabdCIBPHBsΔ¹⁵³ (SEQ ID NO: 82) with codon usage optimized for expression in human cells.

SEQ ID NO: 115 is the nucleotide sequence of a synthetic gene encoding NIgKaFRBHBsΔ⁹⁸ (SEQ ID NO: 83) with codon usage optimized for expression in yeast.

SEQ ID NO: 116 is the nucleotide sequence of a synthetic gene encoding NIgKaFRBHBsΔ⁹⁸ (SEQ ID NO: 83) with codon usage optimized for expression in human cells.

SEQ ID NO: 117 is the nucleotide sequence of a synthetic gene encoding NIgKNCFRBHBsΔ¹⁵³ (SEQ ID NO: 84) with codon usage optimized for expression in yeast.

SEQ ID NO: 118 is the nucleotide sequence of a synthetic gene encoding NIgKNCFRBHBsΔ¹⁵³ (SEQ ID NO: 84) with codon usage optimized for expression in human cells.

The following sequences provide non-limiting examples of amino acid sequences that can be used as targeting domains and connected to a modified HBsAg protein as described herein (e.g., at the N-terminus, internally, and/or at the C-terminus) with or without one or more linker peptides.

SEQ ID NO: 70 is an albumin-binding domain peptide Myeloperoxidase (MPO)-heavy chain, sequence from 425-454: RLATELKSLNPRWDGERLYQEARKIVGAMV (Tiruppathi et al. PNAS 101:7699-7704, 2004).

SEQ ID NO: 71 is the signal peptide sequences of the IgK leader peptide: METDTLLLWVLLLWVPGSTĜD, which is cleaved at GAD. The IgK leader peptide may be used to target the synthesis of a polypeptide to the endoplasmic reticulum (ER).

SEQ ID NO: 72 is the plant signal peptide from soybean vegetative storage vspA: MAMKVLVFFVATILVAWQ̂CHT (Sojikul P et al. PNAS 100: 2209-2214, 2003).

However, it should be appreciated that one or more alternative and/or additional targeting sequences may be used. It also should be appreciated that the selection of targeting sequence (if one is used at all) is a function of the cell, tissue, or organ that is to be targeted by the nanoparticle. It should be appreciated that the targeting sequences provided herein in conjunction with a linker peptide (e.g., one or more GGGGS sequences, SEQ ID NO: 55) may be used without linker peptides or with one or more different linker peptides in alternative chimeric fusion proteins of the invention. It also should be appreciated that any of the targeting peptides described herein may be used to target a nanoparticle without forming a chimeric protein with the modified HBsAg protein. For example, in some embodiments, a nanoparticle may be formed using a modified HBsAg protein as described herein along with one or more separate targeting peptides that are also incorporated into the membrane of the nanoparticle (e.g., as free targeting peptides or used to another protein that is incorporated into the membrane).

It should be appreciated that a fusion protein described herein comprising the fusion of two separate protein sequences may have one (or a few) amino acid deletions and/or substitutions at the fusion junction in order to accommodate nucleic acid sequence requirements (e.g., to avoid a restriction site, a hypersensitivity site, or other undesirable sequence), expression requirements (e.g., to move a Met encoding ATG to the front of the sequence or to remove the N-terminal Met of one of the fusion partners), and/or for any other reason that may be helpful in assembling the fusion protein or the gene encoding the fusion protein.

It should be appreciated that aspects of the invention may include one or more of the specific sequences described herein (e.g., in a recombinant or fusion protein, in a nanoparticle, etc., or any combination thereof). However, aspects of the invention are not limited to the specific sequences described herein as described in more detail herein. It should be appreciated that nucleic acids, polypeptides, and/or proteins of the invention may be made using any suitable recombinant and/or synthetic techniques as aspects of the invention are not limited in this respect.

### DETAILED DESCRIPTION

To overcome the problems of systemic delivery, several nanotechnology-enabled drug delivery systems (DSS) have been developed over the past few years to enable targeted delivery of a therapeutic substances to a specific part of the body of a subject in need of treatment. Nanoparticles currently in use or currently being developed are inorganic nanoparticles (metal oxide- and non-oxide ceramics, calcium phosphate, gold, silicate, magnetic particles), polymer nanoparticles (natural or biocompatible synthetic polymers, polymer-drug and polymer-protein conjugates, polymeric micelles), solid lipid nanoparticles, nanocrystals and liposomes.

The invention provides for a novel therapeutic and/or diagnostic system based on biological nanoparticles. Aspects of the invention relate to hollow nanoparticles that have the capacity to contain one or more substances. In particular, aspects of the invention relate to nanoparticles that can be used to store and/or deliver one or more substances. In certain embodiments, the hollow nanoparticles allow encapsulation of therapeutic or diagnostic agents in their inner space.

In some aspects, the invention provides nanoparticles comprising recombinant proteins (e.g., chimeric fusion proteins) capable of self-assembling into nanoparticles. In some embodiments, chimeric fusion proteins have the formula: AbCdE. In particular aspects of the invention, A and E are targeting domains, C is a domain favoring self assembly, and b and d are linker peptides. In certain embodiments, the chimeric fusion proteins may comprise fewer domains than AbCdE or any combination thereof, provided the chimeric fusion protein comprises at least the (C) domain favoring self assembly.

Aspects of the invention relate to novel nanoparticles based on modified forms of self-assembling viral particle proteins. In particular, aspects of the invention relate to novel self-assembling deletion variants of the Hepatitis B Surface Antigen (HBsAg). Certain hollow nanoparticles provided herein comprise a modified Hepatitis B Surface Antigen S protein domain (HBsAG(S) protein domain) favoring self assembly. In some embodiments, the HBsAg(S) protein domain is truncated, wherein the truncation comprises an amino-terminal deletion of at least one transmembrane domain of the S protein.

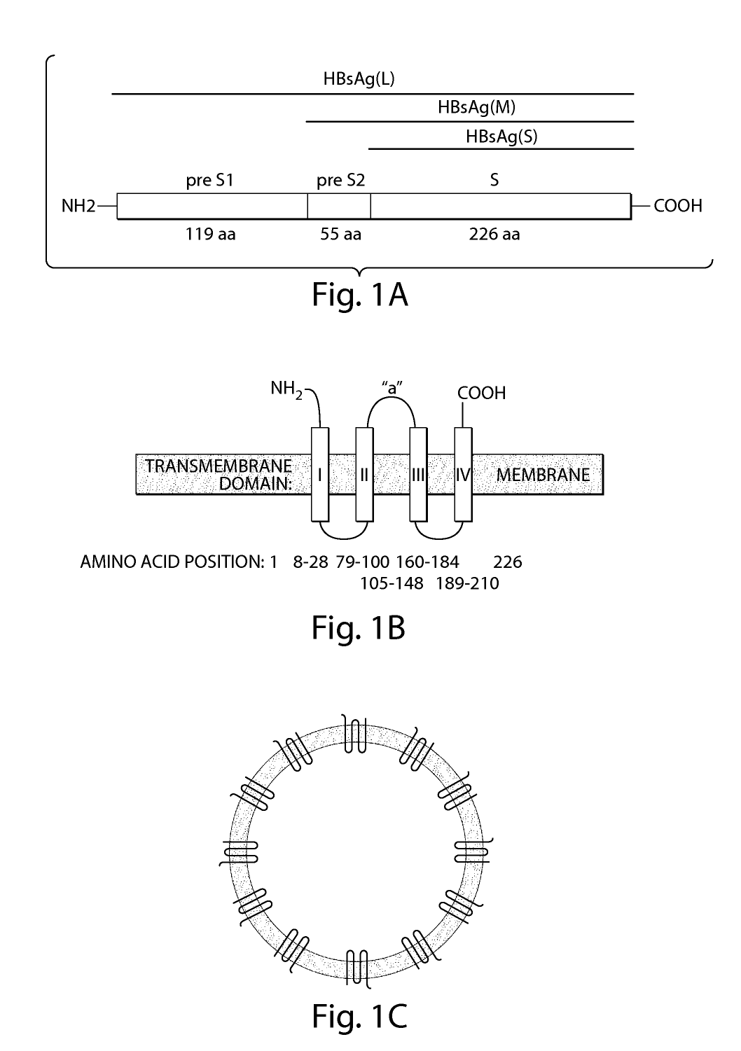

The normal HBsAg(S) protein is one of three surface proteins that are present in the envelope surrounding a normal hepatitis B virus particle. The three surface proteins are the HBsAg large (L), middle (M), and small (S) proteins. These proteins are all encoded in a single open reading frame. The different proteins result from translation starting at different in-frame start codons of the same mRNA. FIG. 1A shows the HBsAg gene structure encoding three protein domains, the pre-S1, pre-S2, and S domains. As illustrated in FIG. 1A, the L protein consists of the pre-S1, pre-S2, and S domains; the M protein consists of the pre-S2 plus the S domain; and the S protein consists of only the S domain. Accordingly, HBsAg(L) consists of the S protein (226 amino acid residues), the Pre-S2 (55 amino acid residues) and Pre-S1 (108 amino acid residues (subtype y) or 119 amino acid residues (subtype d)). HBsAg proteins may include the hepatic cell albumin recognition site contained at positions 3 to 77 in the Pre-S1 region (subtype y) or the hepatic cell recognition site contained in the Pre-S2 region. The HBsAg(S) protein contains four transmembrane helices (domains) that are located between amino acid residues 8-28 (domain I), 79-100 (domain II), 160-184 (domain III) and 189-210 (domain IV) as shown in FIG. 1B. The numbering of the amino acid residues of the S protein corresponds to the full-length amino acid sequence of a normal S protein (226 amino acid residues), starting with position 1 corresponding to the N-terminal Methionine of the S protein (e.g., as illustrated in the Figures and sequences provided herein). Domains I and II are considered amino-terminal, domains III and IV are considered carboxy-terminal for the purpose of this application. HBsAg(S) additionally contains a region between transmembrane domains II and III, which is referred to as the “a” determinant region (amino acids 105-148 of the S protein). The “a” determinant comprises sites of major HB viral antigens. Accordingly, the N- and C-termini and the second hydrophilic region, which bears the major B-cell antigenic determinants (the “a” determinant region, amino acid residues 124-147), are external. A described herein, the four transmembrane helices are located between amino acid residues 8-28 (domain I), 79-100 (domain II), 160-184 (domain III) and 189-210 (domain IV) based on the Stirk model for the small S protein, HBsAg(S). However, it should be appreciated that in some embodiments a deletion of a domain (e.g., domain I, domain II, domain III, or domain IV) may be a deletion of an amino acid sequence that extends to within approximately 5 amino acids (e.g., 10, 9, 8, 7, 6, 5, 4, 3, 2, or 1 amino acids) of one or both of the ends of the domain as defined herein. It should be appreciated that the deletion may be within approximately 5 amino acids N-terminal or C-terminal to the end of the domain as defined herein (e.g., to within approximately 5 amino acids N-terminal or C-terminal of any of positions 8, 28, 79, 100, 160, 184, 189, and 210). However, deletions may extend to other positions as described herein.

Aspects of the invention relate to modified HBsAg(S) proteins, wherein the modification includes the removal of one or more of the N-terminal domains (e.g., domain I, domain II, or both domains I and II) or portions thereof. According to aspects of the invention, HBsAg(S) proteins lacking one or more of the N-terminal domains or portions thereof can self-assemble to form nanoparticles that can be used for delivering one or more agents as described herein.

Hollow nanoparticles comprising Hepatitis B Surface Antigen proteins that self-assemble have been described previously, for example US 2005-0181064, US 2006-0088536, US 2006-0141042, US 2007-0059746, and PCT/JP2007/065646. However, the hollow nanoparticles previously described comprise Hepatitis B Surface Antigen L protein HBsAg(L), which includes the Pre-S1 and Pre-S2 region and the S-protein. The previously described HBsAg(L) proteins comprise deletions within the pre-S region (Pre-S1 and Pre-S2), deletions of the ‘a’ determinant (amino acids 105-148 of the S protein), and deletions of the carboxy-terminus of the S protein (amino acids 154-226). However, previous studies reported that certain N-terminal regions of the Pre-S region may not be deleted without a significant loss of expression of the modified HBsAg(L) proteins in eukaryotic expression systems and that certain amino-terminal regions within the S protein (for example amino-terminal transmembrane region 1: amino acids 8-26, and amino-terminal transmembrane region 2: amino acids 80-98) are deemed critical for self-assembly and should not be deleted or substituted. Retaining the amino-terminal portions of the S protein means however, that these particles retain some or all of the major T-cell epitopes, which are located between N-terminal amino acids 1-100, making these particles problematic for drug delivery by raising an immune response in the subject receiving the nanoparticles.

Based on the teachings in the art it is notable that the Hepatitis B Surface Antigen S proteins HBsAg(S) having a truncation of at least one N-terminal transmembrane domain, and chimeric fusion proteins thereof described herein (having, for example, the general formula AbCdE), maintain their ability to self-assemble and form nanoparticles, and are well expressed in eukaryotic expression systems without the need of particular Pre-S sequences to provide sufficient expression.

Accordingly, aspects of the invention provide hollow nanoparticles comprising recombinant HBsAg(S) proteins having a domain favoring self-assembly. In certain embodiments, the recombinant proteins comprise Hepatitis B Surface Antigen S proteins HBsAg(S) having a truncation of at least one N-terminal transmembrane domain. In some embodiments, at least amino acid residues 8-28 (domain I), or at least amino acid residues 79-100 (domain II) of HBsAg(S) are deleted. In some embodiments, any other amino acid residue of HBsAg(S) may be deleted or modified (e.g., amino acids between transmembrane domains I, II, III, IV, within the transmembrane domains I, II, III, IV, within the ‘a’ determinant region) including, for example, the entire ‘a’ determinant region (amino acids 105-148 of the S protein), and within the C-terminus, amino acids 154-226, provided the resulting chimeric fusion protein is still capable of self-assembly.

Nanoparticles comprising HBsAg(S) proteins having an amino-terminal deletion of at least one transmembrane domain or chimeric fusion proteins thereof have several advantages over those described previously. For example, nanoparticles comprising modified HBsAg(S) proteins of the invention may be less immunogenic (due to the lack of amino-terminal T-cell epitopes), smaller and thus more capable of reaching target cells or tissue, easier to load with therapeutic or diagnostic agents, and/or more flexible for fusing to additional functional domains, such as tissue-specific targeting domains, at the amino-terminus, carboxy-terminus or within the modified HBsAg(S) sequence. In some embodiments, chimeric fusion proteins comprising HBsAg(S) proteins having an amino-terminal deletion of at least one transmembrane domain comprise less than 50% of viral HBsAg(S) protein sequence in the total chimeric fusion protein yet still maintain self-assembly capability. In certain embodiments, the viral HBsAg(S) protein sequence in the chimeric fusion protein is reduced to such an extent that the self-assembled hollow nanoparticle comprising the chimeric fusion protein is essentially non-immunogenic.

It should be appreciated that additional amino acids/peptides (or other non-peptide molecules) may be conjugated (for example, covalently or non-covalently) to the surface of the nanoparticle (for example, to a protein or lipid or other membrane component).

In some embodiments, a transmembrane protein of the invention is a modified HBsAg protein. According to the invention, useful nanoparticles can be produced by using an HBsAg variant that lacks one or more N-terminal transmembrane domains of the S domain (for example, transmembrane domains I and/or II of FIG. 1) as described herein. In some embodiments, a nanoparticle may be produced using only an S domain of the HBsAg that lacks one or more N-terminal transmembrane domains (for example, transmembrane domains I and/or II of FIG. 1). However, it should be appreciated that nanoparticles also may be produced using a mix of HBsAg chimeric fusion proteins, such that some may lack one or more N-terminal transmembrane domains, while others may lack one or more C-terminal transmembrane domains (for example amino acids 154-226), or may lack one C-terminal and one N-terminal transmembrane domain, or may lack other amino acid residues, such as those comprising the ‘a’ determinant, others that may additionally comprise targeting domains, or yet others that may be wild-type. In addition, in some embodiments, the HBsAg molecule may further comprise sequences of the pre-S1 and/or pre-S2 region of HBsAg(L). Such a sequence may be, for example, SEQ ID NO: 54 comprising a domain of the preS1 involved in hepatitis B virus attachment to cell membrane receptors and C-terminal or N-terminal linker peptides. Such pre-S 1 domains may be linked to the N-terminus or C-terminus of the HBsAg molecule, for example according to the formula AbCdE, wherein C is the HBsAg domain favoring self-assembly and A or E are preS1 domains linked to the termini of the HBsAg domain. It should be appreciated that the pre-S1 sequences may be linked without a linker or with a different linker (e.g., different than the GGGGS (SEQ ID NO: 55) linker peptide). In some embodiments, one or more preS1 domains may replace part or all of the ‘a’ determinant region (amino acids 105-148 of the S protein).

Non-limiting examples of modified HBsAg proteins of the invention, including chimeric fusion proteins of the invention are illustrated in Table 1.

TABLE 1

| SEQ | | | |
|---|---|---|---|
| ID | | | |
| NO: | SEQUENCE | NAME | FEATURE |
| 1 | MDYQGMLPVCPLIPGSSTTSTGPCRTCMTTAQGTSMYPSC | HBsΔ⁹⁸ | N-terminal deletion |
| | CCSEPSDRNCTCIPITSSWAFGKELWEWASARFNWLSLLV | | of amino acids 1-98; |
| | PFVQWFVGLSPTVWLSVIWMMWYWGPSLYSILNPFLPLLP | | immune-escape |
| | IFFCLWVYI | | mutations within |
| | | | “a” determinant: |
| | | | Thr140Ser, |
| | | | Lys141Glu, |
| | | | Gly145Arg |
| 57 | MDYQGMLPVCPLIPGSSTTSTGPCRTCMTTAQGTSMYPSC | HBsΔ⁹⁸FH | N-terminal deletion |
| | CCSEPSDRNCTCIPITSSWAFGKFLWEWASARFNWLSLLV | | of amino acids 1-98; |
| | PFVQWFVGLSPTVWLSVIWMMWYWGPSLYSILNPFLPLLP | | C-terminal Flag- |
| | IFFCLWVYIDYKDDDDKVDHHHHHH | | and His-tag, |
| | | | immune-escape |
| | | | mutations within |
| | | | “a” determinant: |
| | | | Thr140Ser, |
| | | | Lys141Glu, |
| | | | Glyl45Arg |
| 60 | METDTLLLWVLLLWVPGSTGDDYQGMLPVCPLIPGSSTTS | NIgKHBsΔ⁹⁸FH | N-terminal deletion |
| | TGPCRTCMTTAQGTSMYPSCCCSEPSDRNCTCIPITSSWA | | of amino acids 1-98; |
| | FGKFLWEWASARFNWLSLLVPFVQWFVGLSPTVWLSVIWM | | N-terminal IgK |
| | MWYWGPSLYSILNPFLPLLPIFFCLWVYIDYKDDDDKVDH | | signal peptide; C- |
| | HHHHH | | terminal Flag- and |
| | | | His-tag, immune- |
| | | | escape mutations |
| | | | within “a” |
| | | | determinant: |
| | | | Thr140Ser, |
| | | | Lys141Glu, |
| | | | Glyl45Arg |
| 2 | MSCCCSEPSDRNCTCIPITSSWAFGKFLWEWASARFNWLSLLVP | HBsΔ¹⁵³ | N-terminal deletion |
| | FVQWFVGLSPTVWLSVIWMMWYWGPSLYSILNPFLPLLPIFFCL | | of amino acids 1- |
| | WVYI | | 153; immune-escape |
| | | | mutations within |
| | | | “a” determinant: |
| | | | Thr140Ser, |
| | | | Lys141Glu, |
| | | | Gly145Arg |
| 58 | MSCCCSEPSDRNCTCIPITSSWAFGKFLWEWASARFNWLSLLVP | HBsΔ¹⁵³FH | N-terminal deletion |
| | FVQWFVGLSPTVWLSVIWMMWYWGPSLYSILNPFLPLLPIFFCL | | of amino acids 1- |
| | WVYIDYKDDDDKVDHHHHHH | | 153; C-terminal |
| | | | Flag- and His-tag, |
| | | | immune-escape |
| | | | mutations within |
| | | | “a” determinant: |
| | | | Thr140Ser, |
| | | | Lys141Glu, |
| | | | Gly145Arg |
| 63 | METDTLLLWVLLLWVPGSTGDSCCCSEPSDRNCTCIPITSSWAF | NIgKHBsΔ¹⁵³FH | N-terminal deletion |
| | GKFLWEWASARFNWLSLLVPFVQWFVGLSPTVWLSVIWMMWYWG | | of amino acids 1- |
| | PSLYSILNPFLPLLPIFFCLWVYIDYKDDDDKVDHHHHHH | | 153; N-terminal IgK |
| | | | signal peptide; C- |
| | | | terminal Flag- and |
| | | | His-tag, immune- |
| | | | escape mutations |
| | | | within “a” |
| | | | determinant: |
| | | | Thr140Ser, |
| | | | Lys141Glu, |
| | | | Gly145Arg |
| 3 | MPLGFFPDHQLDPAFGANSNNPDWDFNPGGGGSGGGGSDYQGML | NHep1HBsΔ⁹⁸ | N-terminal deletion |
| | PVCPLIPGSSTTSTGPCRTCMTTAQGTSMYPSCCCSEPSDRNCT | | of amino acids 1-98; |
| | CIPITSSWAFGKFLWEWASARFNWLSLLVPFVQWFVGLSPTVWL | | N-terminal Pre-S1 |
| | SVIWMMWYWGPSLYSILNPFLPLLPIFFCLWVYI | | domain facilitating |
| | | | cell membrane |
| | | | receptor attachment |
| 67 | METDTLLLWVLLLWVPGSTGDPLGFFPDHQLDPAFGANSNNPDW | NIgKHep1Cabd | N-terminal deletion |
| | DFNPGGGGSGGGGSDYQGMLPVCPLIPGSSTTSTGPCRTCMTTA | HBsΔ⁹⁸ | of amino acids 1-98; |
| | QGTSMYPSCCCSEPSDRNCTCIPITSSWAFGKFLWEWASARFNW | | N-terminal IgK |
| | LSLLVPFVQWFVGLSPTVWLSVIWMMWYWGPSLYSILNPFLPLL | | signal peptide; N- |
| | PIFFCLWVYIGGGGSGGGGSRLATELKSLNPRWDGERLYQEARK | | terminal Pre-S1 |
| | IVGAMV | | domain facilitating |
| | | | cell membrane |
| | | | receptor attachment; |
| | | | C-terminal albumin- |
| | | | binding domain |
| 4 | MDYQGMLPVCPLIPGSSTTSTGPCRICMTTAQGTSMYPSCCCSE | CHep1HBsΔ⁹⁸ | N-terminal deletion |
| | PSDRNCTCTPITSSWAFGKFLWEWASARFNWLSLLVPFVQWFVG | | of amino acids 1-98; |
| | LSPTVWLSVIWMMWYWGPSLYSILNPFLPLLPIFFCLWVYIGGG | | C-terminal Pre-S1 |
| | GSGGGGSPLGFFPDHQLDPAFGANSNNPDWDFNP | | domain facilitating |
| | | | cell membrane |
| | | | receptor attachment |
| 68 | METDTLLLWVLLLWVPGSTGDGGGGSGGGGSRLATELKSLNPRW | NIgKabd | N-terminal deletion |
| | DGERLYQEARKIVGAMVDYQGMLPVCPLIPGSSTTSTGPCRTCM | CHep1HBsΔ⁹⁸ | of amino acids 1-98; |
| | TTAQGTSMYPSCCCSEPSDRNCTCIPITSSWAFGKFLWEWASAR | | N-terminal IgK |
| | FNWLSLLVPFVQWFVGLSPTVWLSVIWMMWYWGPSLYSILNPFL | | signal peptide; N- |
| | PLLPIFFCLWVYIGGGGSGGGGSPLGFFPDHQLDPAFGANSNNP | | terminal albumin- |
| | DWDFNP | | binding domain; C- |
| | | | terminal Pre-S1 |
| | | | domain facilitating |
| | | | cell membrane |
| | | | receptor attachment |
| 5 | MDYQGMLPVCPLIPGSSTTSTGPCRTCMTTAQGTSMYPSCCCSE | αHep1HBsΔ⁹⁸ | N-terminal deletion |
| | GGGGSPLGFFPDHQLDPAFGANSNNPDWDFNPGGGGSCIPITSS | | of amino acids 1-98; |
| | WAFGKFLWEWASARFNWLSLLVPFVQWFVGLSPTVWLSVIWMMW | | inserted Pre-S1 |
| | YWGPSLYSILNPFLPLLPIFFCLWVYI | | domain replacing |
| | | | the second loop of |
| | | | the “a” determinant |
| 69 | METDTLLLWVLLLWVPGSTGDDYQGMLPVCPLIPGSSTTSTGPC | NIgKαHep1 | N-terminal deletion |
| | RTCMTTAQGTSMYPSCCCSEGGGGSPLGFFPDHQLDPAFGANSN | CabdHBsΔ⁹⁸ | of amino acids 1-98; |
| | NPDWDFNPGGGGSCIPITSSWAFGKFLWEWASARFNWLSLLVPF | | N-terminal IgK |
| | VQWFVGLSPTVWLSVIWMMWYWGPSLYSILNPFLPLLPIFFCLW | | signal peptide; C- |
| | VYIGGGGSGGGGSRLATELKSLNPRWDGERLYQEARKIVGAMV | | terminal albumin- |
| | | | binding domain; |
| | | | inserted Pre-S1 |
| | | | domain replacing |
| | | | the second loop of |
| | | | the “a” determinant |
| 6 | MPLGFFPDHQLDPAFGANSNNPDWDFNPGGGGSDYQGMLPVCPL | NαHep1HBsΔ⁹⁸ | N-terminal deletion |
| | IPGSSTTSTGPCRTCMTTAQGTSMYPSCCCSEGGGGSPLGFFPD | | of amino acids 1-98; |
| | HQLDPAFGANSNNPDWDFNPGGGGSCIPITSSWAFGKFLWENAS | | N-terminal and |
| | ARFNWLSLLVPFVQWFVGLSPTVWLSVIWMMWYWGPSLYSILNP | | inserted Pre-S1 |
| | FLPLLPIFFCLWVYI | | domain replacing |
| | | | the second loop of |
| | | | the “a” determinant |
| 73 | METDTLLLWVLLLWVPGSTGDPLGFFPDHQLDPAFGANSNNPDW | NIgKαHep1 | N-terminal deletion |
| | DFNPGGGGSDYQGMLPVCPLIPGSSTTSTGPCRTCMTTAQGTSM | CabdHBsΔ⁹⁸ | of amino acids 1-98; |
| | YPSCCCSEGGGGSPLGFFPDHQLDPAFGANSNNPDWDFNPGGGG | | N-terminal IgK |
| | SCIPITSSWAFGKFLWEWASARFNWLSLLVPFVQWFVGLSPTVW | | signal peptide; N- |
| | LSVIWMMWYWGPSLYSILNPFLPLLPIFFCLWVYIGGGGSGGGG | | terminal and |
| | SRLATELKSLNPRWDGERLYQEARKIVGAMV | | inserted Pre-S1 |
| | | | domain replacing |
| | | | the second loop of |
| | | | the “a” determinant; |
| | | | C-terminal albumin- |
| | | | binding domain |
| 7 | MDYQGMLPVCPLIPGSSTTSTGPCRTCMTTAQGTSMYPSCCCSE | αCHep1HBsΔ⁹⁸ | N-terminal deletion |
| | GGGGSPLGFFPDHQLDPAFGANSNNPDWDFNPGGGGSCIPITSS | | of amino acids 1-98; |
| | WAFGKFLWEWASARFNWLSLLVPFVQWFVGLSPTVWLSVIWMMW | | C-terminal and |
| | YWGPSLYSILNPFLPLLPIFFCLWVYIGGGGSPLGFFPDHQLDP | | inserted Pre-S1 |
| | AFGANSNNPDWDFNP | | domain replacing |
| | | | the second loop of |
| | | | the “a” determinant |
| 74 | METDTLLLWVILLWVPGSTGDGGGGSGGGGSRLATELKSLNPRW | NIgKabdαC | N-terminal deletion |
| | DGERLYQEARKIVGAMVDYQGMLPVCPLIPGSSTTSTGPCRTCM | Hep1HBsΔ⁹⁸ | of amino acids 1-98; |
| | TTAQGTSMYPSCCCSEGGGGSPLGFFPDHQLDPAFGANSNNPDW | | N-terminal IgK |
| | DFNPGGGGSCIPITSSWAFGKFLWEWASARFNWLSLLVPFVQWF | | signal peptide; N- |
| | VGLSPTVWLSVIWMMWYWGPSLYSILNPFLPLLPIFFCLWVYIG | | terminal albumin- |
| | GGGSPLGFFPDHQLDPAFGANSNNPDWDFNP | | binding domain; C- |
| | | | terminal and |
| | | | inserted Pre-S1 |
| | | | domain replacing |
| | | | the second loop of |
| | | | the “a” determinant |
| 8 | MPLGFFPDHQLDPAFGANSNNPDWDFNPGGGGSGGGGSDYQGML | NHep1HBsΔ⁹⁸ | N-terminal deletion |
| | PVCPLIPGSSTTSTGPCRTCMTTAQGTSMYPSCCCSEPSDRNCT | KDR | of amino acids 1-98; |
| | CIPITSSWAFGKFLWEWASARFNWLSLLVPFVQWFVGLSPTVWL | | Pre-S1 domain and |
| | SVIWMMWYWGPSLYSILNPFLPLLPIFFCLWVYIAAAASGDSRV | | VEGF-R¹ binding |
| | CWEDSWGGEVCFRYDP | | sequence, |
| | | | facilitating cell |
| | | | membrane receptor |
| | | | attachment |
| 75 | METDTLLLWVLLLWVPGSTGDPLGFFPDHQLDPAFGANSNNPDW | NIgKHep1Cabd | N-terminal deletion |
| | DFNPGGGGSGGGGSDYQGMLPVCPLIPGSSTTSTGPCRTCMTTA | HBsΔ⁹⁸KDR | of amino acids 1-98; |
| | QGTSMYPSCCCSEPSDRNCTCIPITSSWAFGKFLWEWASARFNW | | N-terminal IgK |
| | LSLLVPFVQWFVGLSPTVWLSVIWMMWYWGPSLYSILNPFLPLL | | signal peptide; Pre- |
| | PIFFCLWVYIAAAASGDSRVCWEDSWGGEVCFRYDPGGGGSGGG | | S1 domain and |
| | GSRLATELKSLNPRWDGERLYQEARKIVGAMV | | VEGF-R¹ binding |
| | | | sequence, |
| | | | facilitating cell |
| | | | membrane receptor |
| | | | attachment; C- |
| | | | terminal albumin- |
| | | | binding domain |
| 9 | MPLGFFPDHQLDPAFGANSNNPDWDFNPGGGGSGGGGSSWAFGK | NHep1HBsΔ¹⁵³ | N-terminal deletion |
| | FLWEWASARFNWLSLLVPFVQWFVGLSPTVWLSVIWMMWYWGPS | | of amino acids 1- |
| | LYSILNPFLPLLPIFFCLWVYI | | 153; N-terminal |
| | | | Pre-S1 domain |
| 76 | METDTLLLWVLLLWVPGSTGDPLGFPPDHQLDPAFGANSNNPDW | NIgKHep1Cabd | N-terminal deletion |
| | DFNPGGGGSGGGGSSWAFGKFLWEWASARFNWLSLLVPFVQWFV | HBSsΔ¹⁵³ | of amino acids 1- |
| | GLSPTVWLSVIWMMWYWGPSLYSILNPFLPLLPIFFCLWVYIGG | | 153; N-terminal IgK |
| | GGSGGGGSRLATELKSLNPRWDGERLYQEARKIVGAMV | | signal peptide; N- |
| | | | terminal Pre-S1 |
| | | | domain; C-terminal |
| | | | albumin-binding |
| | | | domain |
| 10 | MSSWAFGKFLWEWASARFNWLSLLVPFVQWFVGLSPTVWLSVIW | CHep1HBsΔ¹⁵³ | N-terminal deletion |
| | MMWYWGPSLYSILNPFLPLLPIFFCLWVYIGGGGSGGGGSPLGF | | of amino acids 1- |
| | FPDHQLDPAFGANSNNPDWDFNP | | 153; N-terminal IgK |
| | | | signal peptide; C- |
| | | | terminal Pre-S1 |
| | | | domain |
| 77 | METDTLLLWVLLLWVPGSTGDGGGGSGGGGSRLATELKSLNPRW | NIgKabdCHep1 | N-terminal deletion |
| | DGERLYQEARKIVGAMVSSWAFGKFLWEWASARFNWLSLLVPFV | HBsΔ¹⁵³ | of amino acids 1- |
| | QWFVGLSPTVWLSVIWMMWYWGPSLYSILNPFLPLLPIFFCLWV | | 153; N-terminal IgK |
| | YIGGGGSGGGGSPLGFFPDHQLDPAFGANSNNPDWDFNP | | signal peptide; N- |
| | | | terminal albumin- |
| | | | binding domain; C- |
| | | | terminal Pre-S1 |
| | | | domain |
| 11 | MPLGFFPDHOLDPAFGANSNNPDWDFNPGGGGSSWAFGKFLWEW | NCHep1HBsΔ¹⁵³ | N-terminal deletion |
| | ASARFNWLSLLVPFVQWFVGLSPTVWLSVIWMMWYWGPSLYSIL | | of amino acids 1- |
| | NPFLPLLPIFFCLWVYIGGGGSPLGFFPDHQLDPAFGANSNNPD | | 153; N- and C- |
| | WDFNP | | terminal Pre-S1 |
| | | | domain |
| 78 | METDTLLLWVLLLWVPGSTGDPLGFFPDHQLDPAFGANSNNPDW | NIgkNCHep1 | N-terminal deletion |
| | DFNPGGGGSGGGGSSWAFGKFLWEWASARFNWLSLLVPFVQWFV | HBsΔ¹⁵³ | of amino acids 1- |
| | GLSPTVWLSVIWMMWYWGPSLYSILNPFLPLLPIFFCLWVYIGG | | 153; N-terminal IgK |
| | GGSGGGGSPLGFFPDHQLDPAFGANSNNPDWDFNP | | signal peptide; N- |
| | | | and C-terminal Pre- |
| | | | S1 domain |
| 12 | MPNNNKILVPKVSGLQYRVFRGGGGSDYQGMLPVCPLIPGSSTT | NαL1HBsΔ¹⁵³ | N-terminal deletion |
| | STGPCRTCMTTAQGTSMYPSCCCSEGGGGSLYIKGSGSTANLAS | | of amino acids 1- |
| | SNYFPTGGGGSCIPITSSWAFGKFLWEWASARFNWLSLLVPFVQ | | 153; N-terminal |
| | WFVGLSPTVWLSVIWMMWYWGPSLYSILNPFLPLLPIFFCLWVY | | HPV-16, L1⁸¹⁻¹⁰⁰ |
| | I | | and L1³⁰¹⁻³²⁰ domains |
| | | | replacing the |
| | | | second loop of |
| | | | the “a” determinant |
| 79 | METDTLLLWVLLLWVPGSTGDPNNNKILVPKVSGLQYRVFRGGG | NIgKαL1Cabd | N-terminal deletion |
| | GSDYQGMLPVCPLIPGSSTTSTGPCRTCMTTAQGTSMYPSCCCS | HBsΔ¹⁵³ | of amino acids 1- |
| | EGGGGSLYIKGSGSTANLASSNYFPTGGGGSCIPITSSWAFGKF | | 153; N-terminal IgK |
| | LWEWASARFNWLSLLVPFVQWFVGLSPTVWLSVIWMMWYWGPSL | | signal peptide; N- |
| | YSILNPFLPLLPIFFCLWVYIGGGGSGGGGSRLATELKSLNPRW | | terminal HPV-16, |
| | DGERLYQEARKIVGAMV | | L1⁸¹⁻¹⁰⁰ and L1³⁰¹⁻³²⁰ |
| | | | domains replacing |
| | | | the second loop of |
| | | | the “a” determinant; |
| | | | C-terminal albumin- |
| | | | binding domain |
| 13 | MPNNNKILVPKVSGLQYRVFRGGGGSSWAFGKFLWEWASARFNW | NCL1HBsΔ¹⁵³ | N-terminal deletion |
| | LSLLVPFVQWFVGLSPTVWLSVIWMMWYWGPSLYSILNPFLPLL | | of amino acids 1- |
| | PIFFCLWVYIGGGGSLYIKGSGSTANLASSNYFPT | | 153; N- and C- |
| | | | terminal HPV-16, |
| | | | L1⁸¹⁻¹⁰⁰ and L1³⁰¹⁻³²⁰ |
| | | | domains |
| 80 | METDTLLLWVLLLWVPGSTGDPNNNKILVPKVSGLQYRVFRGGG | NIgKNCL1 | N-terminal deletion |
| | GSSWAFGKFLWEWASARFNWLSLLVPFVQWFVGLSPTVWLSVIW | HBsΔ¹⁵³ | of amino acids 1- |
| | MMWYWGPSLYSILNPFLPLLPIFFCLWVYIGGGGSLYIKGSGST | | 153; N-terminal IgK |
| | ANLASSNYFPT | | signal peptide; N- |
| | | | and C-terminal |
| | | | HPV-16, L1⁸¹⁻¹⁰⁰ |
| | | | and L1³⁰¹⁻³²⁰ |
| | | | domains |
| 14 | MGRGDSPGGGGSDYQGMLPVCPLIPGSSTTSTGPCRTCMTTAQG | NαIBPHBsΔ⁹⁸ | N-terminal deletion |
| | TSMYPSCCCSEGGGGSPHSRNGGGGSCIPITSSWAFGKFLWEWA | | of amino acids 1-98; |
| | SARFNWLSLLVPFVQWFVGLSPTVWLSVIWMMWYWGPSLYSILN | | integrin binding |
| | PFLPLLPIFFCLWVYI | | domains derived |
| | | | from fibronectin |
| 81 | METDTLLLWVLLLWVPGSTGDGRGDSPGGGGSDYQGMLPVCPLI | NIgKαIBPCabd | N-terminal deletion |
| | PGSSTTSTGPCRTCMTTAQGTSMYPSCCCSEGGGGSPHSRNGGG | HBsΔ⁹⁸ | of amino acids 1-98; |
| | GSCIPITSSWAFGKFLWEWASARFNWLSLLVPFVQWFVGLSPTV | | N-terminal IgK |
| | WLSVIWMMWYWGPSLYSILNPFLPLLPIFFCLWVYIGGGGSGGG | | signal peptide; |
| | GSRLATELKSLNPRWDGERLYQEARKIVGAMV | | integrin binding |
| | | | domains derived |
| | | | from fibronectin; C- |
| | | | terminal albumin- |
| | | | binding domain |
| 15 | MGRGDSPGGGGSSWAFGKFLWEWASARFNWLSLLVPFVQWFVGL | NCIBPHBsΔ¹⁵³ | N-terminal deletion |
| | SPTVWLSVIWMMWYWGPSLYSILNPFLPLLPIFFCLWVYIGGGG | | of amino acids 1- |
| | SPHSRN | | 153; integrin |
| | | | binding domains |
| | | | derived from |
| | | | fibronectin |
| 82 | METDTLLLWVLLLWVPGSTGDGRGDSPGGGGSRLATELKSLNPR | NIgKabdCIBP | N-terminal deletion |
| | WDGERLYQEARKIVGAMVGGGGSGGGGSSWAFGKFLWEWASARF | HBsΔ¹⁵³ | of amino acids 1- |
| | NWLSLLVPFVQWFVGLSPTVWLSVIWMMWYWGPSLYSILNPFLP | | 153; N-terminal IgK |
| | LLPIFFCLWVYIGGGGSPHSRN | | signal peptide; N- |
| | | | terminal albumin- |
| | | | binding domain; |
| | | | integrin binding |
| | | | domains derived |
| | | | from fibronectin |
| 16 | MQLPLATGGGGSDYQGMLPVCPLIPGSSTTSTGPCRTCHTTAQG | NαFRBHBsΔ⁹⁸ | N-terminal deletion |
| | TSMYPSCCCSEGGGGSMQLPLATGGGGSCIPITSSWAFGKFLWE | | of amino acids 1-98; |
| | WASARFNWLSLLVPFVQWFVGLSPTVWLSVIWMMWYWGPSLYSI | | FGR-R² binding |
| | LNPFLPLLPIFFCLWVYIGGGGSGGGGSMQLPLAT | | domain |
| 83 | METDILLLWVLLLWVPGSTGDQLPLATGGGGSDYQGMLPVCPLI | NIgKαFRB | N-terminal deletion |
| | PCSSTTSTGPCRTCMTTAQGTSMYPSCCCSEGGGGSMQLPLATG | HBsΔ⁹⁸ | of amino acids 1-98; |
| | GGGSCIPITSSWAFGKFLWEWASARFNWLSLLVPFVQWFVGLSP | | N-terminal IgK |
| | TVWLSVIWMMWYWGPSLYSILNPFLPLLPIFFCLWVYIGGGGSG | | signal peptide; |
| | GGGSMQLPLAT | | FGR-R² binding |
| | | | domain |
| 17 | MQLPLATGGGGSSWAFGKFLWEWASARFNWLSLLVPFVQWFVGL | NCFRBHBsΔ¹⁵³ | N-terminal deletion |
| | SPTVWLSVIWMMWYWGPSLYSILNPFLPLLPIFFCLWVYIGGGG | | of amino acids 1- |
| | SMQLPLAT | | 153; FGR-R² |
| | | | binding domain |
| 84 | METDTLLLWVLLLWVPGSTGDQLPLATGGGGSSWAFGKFLWEWA | NIgKNCFRB | N-terminal deletion |
| | SARFNWLSLINPFVQWFVGLSPTVWLSVIWMMWYWGPSLYSILN | HBsΔ¹⁵³ | of amino acids 1- |
| | PFLPLLPIFFCLWVYIGGGGSMQLPLAT | | 153; N-terminal IgK |
| | | | signal peptide; |
| | | | FGR-R² binding |
| | | | domain |

¹VEGF-R: vascular endothelial growth factor receptor
²FGF-R: fibroblast growth factor receptor

It should be appreciated that any of the targeting peptides (e.g., Pre-S1, integrin, FGR-R, HPV-16, etc.), leader sequences, detection and/or purification tags, any other functional peptides or domains (e.g., albumin binding domain) illustrated or described herein, or equivalents thereof, may be fused alone, or in combination, at the N-terminus, C-terminus, and/or internally, with any of the chimeric proteins described herein, with or without one or more linker peptides, optionally including additional amino acid changes (e.g., to remove one or more antigenic sequences). It should be appreciated that any of the proteins or peptides may be synthetic, natural, mammalian, non-mammalian, viral, bacterial, plant, yeast, or any combination thereof.

It should be appreciated that the different domains and sequences illustrated in Table 1 may be used in conjunction with different linker peptides (e.g., different from the GGGGS (SEQ ID NO: 55) linker peptide) or without any linker peptides in alternative chimeric proteins of the invention.

It should be appreciated that different modifications may be used to produce modified HBsAg proteins of the invention. In some embodiments, a protein or nanoparticle of the invention has at least one or both of the N-terminal trans-membrane domains of the S protein portion removed. As used herein, removing can mean deleting a part or more of each domain. According to aspects of the invention, by removing one or both of the N-terminal domains of the HBsAg molecule, certain properties may be improved. N-terminal deletions are understood to include full deletions, that include every amino acid from the first N-terminal amino acid of the protein, or partial deletions, that may delete only certain amino acids within the amino-terminal half of the full-length protein. Deletions may comprise one or more amino acids. It should be appreciated that other changes that remove one or more N-terminal sequences also may be used (e.g., mutations etc.). Accordingly, in some embodiments, some or all of amino acid residues 8-28 (domain I), and/or some or all of amino acid residues 79-100 (domain II) of HBsAg(S) are deleted. It should be appreciated that a subset of one or both domains may be removed (e.g., from about residue 8, 9, 10, 11, or 12 to about residue 24, 25, 26, 27, or 28 of domain I, and/or from about residue 79, 80, 81, 82, or 83 to about residue 96, 97, 98, 99, or 100 of domain II). However, more or fewer residues may be deleted and/or modified. In some embodiments, a modified protein of the invention may have an N-terminal truncation of the S protein moiety extending from residue 1 of the S sequence to a residue within domain I, within domain II, or beyond domain II of the S sequence (e.g., including part or all of domain I, also including part or all of domain II, or also including part or all of the “a” region). In some embodiments, a recombinant or chimeric protein of the invention may include a HBsAg(S) domain with a truncation of S amino acids 1-8, 1-20, 1-21, etc., 1-95, 1-96, 1-97, 1-98, 1-99, 1-100, etc., 1-130, 1-131, 1-132, 1-133, 1-134, 1-135, 1-140, 1-141, 1-142, 1-1424, 1-143, 1-143, 1-144, 1-145, 1-146, 1-147, 1-148, 1-149, 1-150, 1-151, 1-152, 1-153, 1-154, 1-155, 1-156, 1-157, 1-158, 1-159, or having more or fewer or intermediate numbers of amino acid deletions. In some embodiments, a protein of the invention may have a deletion of one or more N- and/or O-glycosylation sites. In some embodiments, a protein of the invention may be modified to introduce one or more of pegylation sites or other modifications such as pegylation that can help mask the particles from the immune system, and/or increase the hydrodynamic size of the nanoparticles which may extend their circulatory time. In some embodiments, pegylation or similar modifications can be used to provide water solubility. In some embodiments, one or more internal deletions (e.g., 35-75, 109-153) may be combined with other deletions described herein. In some embodiments, internal amino acids (e.g., amino acids 98-153 and/or amino acids 185-188) can be substituted with a linker sequence that facilitates interaction with a specific cargo: e.g., Gadolinium.

It should be appreciated that any modified S protein moieties described herein may be provided as isolated proteins or may be provided in the form of chimeric fusion proteins linked to one or more N-terminal, internal, and/or C-terminal peptides as described herein.

Variant proteins also may be engineered (for example, via recombinant cloning) to have amino acid additions at the N-terminal, C-terminal, or internal regions, or any combination thereof. For example, one or more antigenic amino acid sequences, targeting amino acid sequences, purification amino acid sequences, or any other additional sequence motif (for example, for stabilization, solubility, etc.) may be included at the N-terminus, C-terminus, or within the body of the protein, or any combination thereof. Non-limiting examples are provided in Table 1.

In certain embodiments, the HBsAg molecule may further comprise sequences for targeting molecules which are N-terminal or C-terminal of the sequences of the HBsAg molecule that lacks one or more N-terminal transmembrane domains. Targeting molecules can be, for example targeting peptides, antibodies, or receptor molecules, or fragments thereof. These molecules may also facilitate membrane translocation by receptor mediated endocytosis or other mechanisms. For example, Pre-S I sequences may be fused to a modified HBsAg molecule to provide targeting to hepatocytes. As another example, an α5β6 integrin peptide may be fused to a modified HBsAg molecule to provide targeting to many tumor tissues and cells. As yet another example, the albumin-binding domain may be fused to a modified HBsAg molecule to provide targeting to many tumor tissues and cells, and also to increase transcytosis and/or to increase uptake in vivo through the gut, for example, in embodiments where the nanoparticle is administered orally. An example of an albumin-binding domain peptide is Myeloperoxidase (MPO)-heavy chain (sequence from 425-454: RLATELKSLNPRWDGERLYQEARKIVGAMV (SEQ ID NO: 70) (Tiruppathi et al. PNAS 101:7699-7704, 2004). In another example, integrin binding domains, such as GRGDSP (SEQ ID NO: 64) and PHSRN (SEQ ID NO: 65) derived from fibronectin, shown here with one or more GGGGS linker peptides (SEQ ID NO: 55), may be fused to a modified HBsAg molecule lacking one or more N-terminal transmembrane domains of the S protein. In yet another example, fibroblast growth factor receptor-binding domains, such as MQLPLAT (SEQ ID NO: 66), shown here with one or more GGGGS linker peptides (SEQ ID NO: 55), may be fused to a modified HBsAg molecule lacking one or more N-terminal transmembrane domains of the S protein.

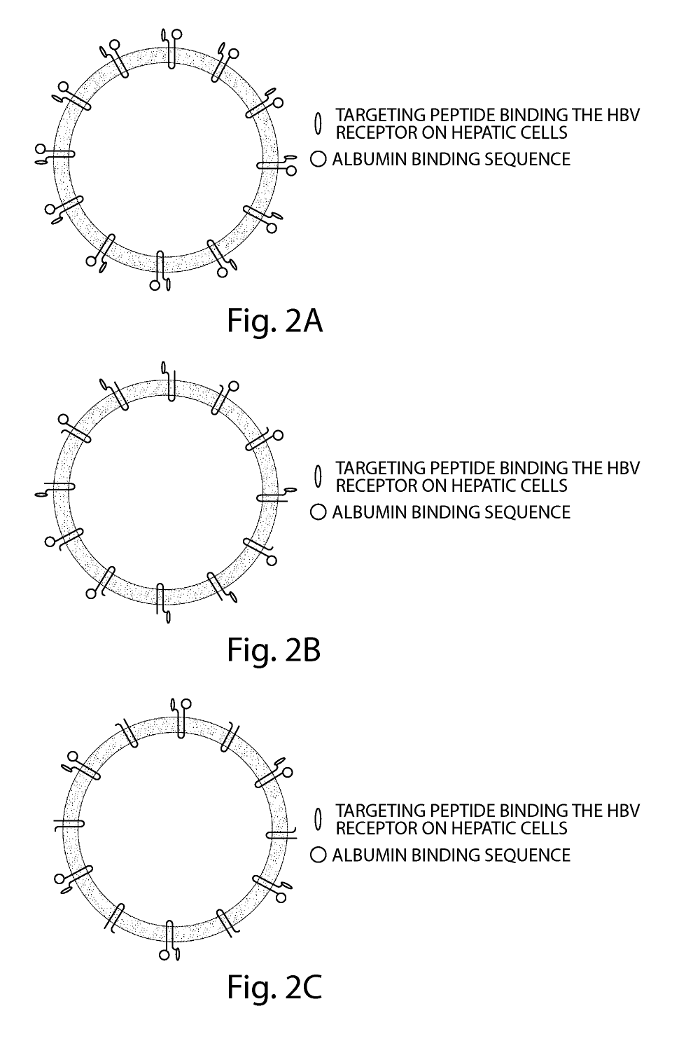

Non-limiting embodiments of modified HBsAg(S) proteins of the invention are provided in Table 1 and in FIG. 2. FIG. 2 shows non-limiting examples of HBsAg nanoparticles of the invention comprising chimeric fusion proteins comprising a truncated Hepatitis B Surface Antigen S domain protein fused to additional functional peptides or protein domains, such as for example targeting peptides and/or albumin binding sequences. The nanoparticle may comprise, for example, a chimeric fusion protein comprising a truncated Hepatitis B Surface Antigen S domain protein fused on both termini to additional functional peptides or protein domains, such as N-terminally to a targeting peptide and C-terminally to an albumin binding sequence, as depicted in FIG. 2A. The nanoparticle may comprise, for example, two (or more) different truncated Hepatitis B Surface Antigen S domain proteins fused to one (or more) different additional functional peptides or protein domains, such as either N-terminally or C-terminally to either a targeting peptide or to an albumin binding sequence, as depicted in FIG. 2B. The nanoparticle may comprise, for example, two (or more) different truncated Hepatitis B Surface Antigen S domain proteins wherein one S domain protein is fused to one (or more) additional functional peptides or protein domains and one is not, as depicted in FIG. 2C. FIG. 2 A-C only depict some illustrative examples. Many more variations are possible with these and other fusion partners.

In certain embodiments, targeting molecules can be protein tags facilitating purification, such as, for example, myc-tag, His-tag (HHHHHH, SEQ ID NO: 53), or FLAG-tag (DYKDDDDK, SEQ ID NO: 52).

In certain embodiments, targeting molecules can be signal peptide sequences for endoplasmic reticulum targeting, such as the IgK leader peptide (METDTLLLWVLLLWVPGSTĜD, SEQ ID NO: 71) which is cleaved at GAD. Other examples of signal peptides are the plant signal peptide from soybean vegetative storage vspA: MAMKVLVFFVATILVAWQ̂CHT, SEQ ID NO: 72) (Sojikul P et al. PNAS 100: 2209-2214, 2003).

Nanoparticles of the invention also may be engineered to include one or more non-peptide targeting molecules (for example, aptamers against target antigens such as MUC-1 or other tumor associated antigens). In some embodiments, nanoparticles are conjugated with aptamers, for example RNA aptamers, that recognize tumor antigens. Such aptamers may, for example, recognize the extracellular domain of the prostate-specific membrane antigen (PSMA), a well characterized antigen expressed on the surface of prostate cancer cells (Farokhzad et al. PNAS: 103: 6315-6320, 2006), or other such cancer-specific antigens.

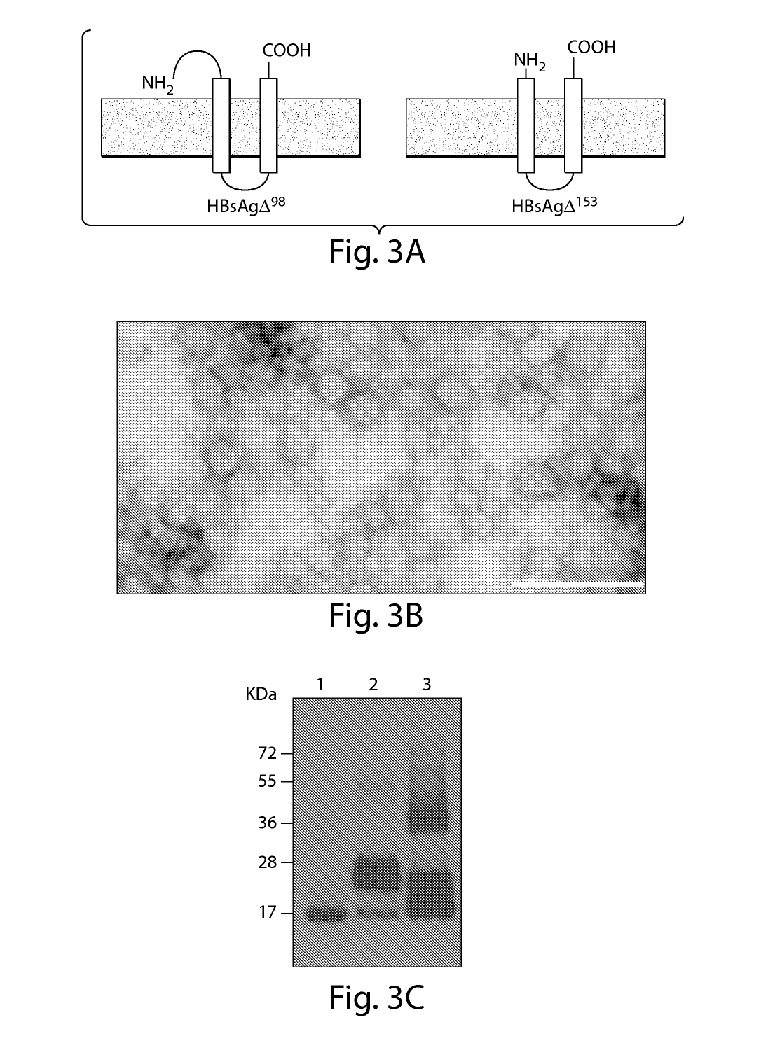

Variant HBsAg proteins also may be engineered, through deletions and/or mutations to have reduced immunogenicity and/or intra-molecular stability. One or more mutations may be introduced in the wild-type amino acid sequence, such as, for example: L8F, K24R, P46T, A50G, Q56P, I57T, S59N, C64S, I68T, C85F, K122R, T125M, P127T, F134Y, T143S, A159G, Y161F, V168A, N207S, I213L (see, for example, SEQ ID NO: 129, FIG. 9A). The name of the amino acid is followed by the position of the amino acid in the wild-type sequence, which is followed by the name of the amino acid of the substitution/mutation introduced. The mutations are either within the ‘a’ determinant region, or outside the ‘a’ determinant region and can affect either immunogenicity or intra-molecular stability of the of the HBsAg(S) molecule, or both. The ‘a’ determinant region comprises the majority of B-cell epitopes and therefore contributes to the immunogenicity of the HBsAg proteins (Jilg W. Vaccine 16 Suppl:S65-S68, 1998). In certain embodiments, variant HBsAg proteins lack the ‘a’ determinant region (as described in FIG. 1) in its entirety (see, for example, FIG. 3A), or they may lack only portions of it, to reduce or completely diminish immunogenicity. For example, variant HBsAg proteins may comprise a deletion of residues 142-148 in the second loop of the “a” determinant. In these variant HBsAg proteins, one, two, three or four of the cysteines, important for the conformation of the tertiary structure of HBsAg, at positions 124, 137, 139, and 149, are conserved. In certain embodiments, one or more mutations may be introduced within the “a” determinant, such as Thr140Ser, and/or Lys141Glu and/or Gly145Arg. In certain embodiments, all or most of the ‘a’ determinant region (amino acids 110-150) is deleted or has been replaced in the variant HBsAg proteins.

Other mutations can be introduced that reduce immunogenicity and/or reduce or enhance intra-molecular stability outside the ‘a’ determinant region. For example, in certain embodiments, modified HBsAg proteins comprise an S domain having a deletion of the first 98 amino-terminal amino acids of the S domain (HBsAg(S)Δ98), thereby removing the major T-cell epitopes that contribute to the immunogenicity of the HBsAg protein. In some embodiments, an HBSAg variant of the invention may be humanized to remove epitopes that are immunogenic in humans. In some embodiments, mutations and/or deletions in the ‘a’ determinant region are combined with amino-terminal deletions, as described herein, and may optionally or additionally be combined with one or more of the following amino acid substitutions: L8F, K24R, P46T, A50G, Q56P, I57T, S59N, C64S, I68T, C85F, K122R, T125M, P127T, F134Y, T143S, A159G, Y161F, V168A, N207S, I213L. In some embodiments, the mutations, deletions and substitutions described herein are further combined with additions of protein sequences, such as targeting peptides or domains or other functional peptides or domains, as described herein. In certain embodiments, the viral HBsAg(S) protein sequence in the chimeric fusion protein is reduced to such an extent that the self-assembled hollow nanoparticle comprising such chimeric fusion protein is essentially non-immunogenic.

Reduced immunogenicity as used herein means that a nanoparticle comprising a HBsAg protein comprising one or more deletions or mutations reducing B cell-mediated and/or T cell-mediated immunogenicity is less immunogenic (for example when administered to a host). For example, in some embodiments it induces a more limited immune reaction in the host (or may be entirely non-immunogenic in the host) as compared to a nanoparticle comprising non-altered or wild-type HBsAg protein.

Nanoparticles may be made by expressing a membrane protein of the invention in a host cell that naturally produces particles (for example, they are budded off and/or released from the host cell, for example by budding off the cell membrane or by partial or complete lysis of the cell). In certain embodiments, signal peptide sequences for targeting the endoplasmic reticulum, such as the IgK leader peptide, can be fused to the HBsAg(S) protein. HBsAg(S) fusion proteins comprising the IgK leader peptide are shown in Table 1, for example, SEQ ID NOs: 60, 63, 67-69, 73-84). In some embodiments, a host cell that expresses a membrane protein of the invention may be artificially lysed (for example, by sonication, mild detergent, other technique, or any combination thereof) to generate particles. Host cells may be prokaryotic or eukaryotic. For example, bacterial, yeast, insect, avian, reptilian, mammalian (for example, human, primate, hamster, mouse, rat, pig, etc.), or other host cells may be used.

In some embodiments, nanoparticles may be isolated from supernatants or cell lysates using appropriate separation and/or concentration techniques. For example, one or more centrifugation steps (e.g., density gradient centrifugation, for example using CsCl, sucrose, and/or other appropriate centrifugation medium) may be used to obtain fractions enriched for nanoparticles. These steps may be repeated to further purify the isolated particles. In some embodiments, one or more dialysis steps are used to further prepare a preparation of isolated nanoparticles. In some embodiments, one or more concentration steps also may be used. Accordingly, different preparations of isolated nanoparticles may have different particle concentrations and/or levels of purification. Non-limiting examples of particle preparation techniques that may be used for the different nanoparticles described herein are provided in the Examples, and also known in the literature (see, for example, the particle preparation techniques described in US Patent Publication No. 2005/0181064, incorporated by reference herein in their entirety).

In some embodiments, nanoparticles may be made by assembling isolated or purified proteins of the invention. In some embodiments, the isolated or purified proteins may be mixed with other proteins or lipids to form nanoparticles. Nanoparticles may be formed by concentration, dialysis, or other suitable technique or any combination thereof. It should be appreciated that proteins of the invention may be isolated or purified from cells that express the proteins using standard purification techniques with our without affinity purification techniques (e.g., for proteins that contain one or more affinity purification tags as described herein). It should be appreciated that proteins of the invention may be over-expressed in any suitable cells from one or more gene sequences under the control of a suitable promoter (e.g., constitutive or inducible). The protein-encoding gene(s) may be located on a replicating plasmid or other vector, or chromosomally integrated, or any combination thereof. The genes may be made recombinantly and/or synthetically using any suitable technique. It also should be appreciated that in some embodiments, polypeptides or proteins of the invention may be expressed in vitro or made synthetically using suitable chemical synthesis steps as aspects of the invention are not limited in this respect.

It should be appreciated that preparations of nanoparticles may be provided in different solutions or buffers. In some embodiments, a solution may be a hypotonic solution or water. The solution or buffer may be pharmaceutically acceptable and/or physiologically compatible. In some embodiments, sterile preparations may be made using sterile processing techniques and/or by sterilizing nanoparticle preparations using appropriate techniques (e.g., filtration, chemicals, irradiation, etc., or any combination thereof).

FIG. 3 provides non-limiting examples of nanoparticles containing truncated HBsAg(S) proteins. FIG. 3A depicts a schematic showing two examples of truncated Hepatitis B Surface Antigen S domain proteins: HBsAgΔ⁹⁸ (having an amino-terminal deletion of the first 98 amino acids of the HBsAg(S) protein of 226 amino acids) and HBsAgΔ¹⁵³ (having an amino-terminal deletion of the first 153 amino acids of the HBsAg(S) protein of 226 amino acids). HBsAgΔ⁹⁸ comprises portions of the ‘a’ determinant region. HBsAgΔ¹⁵³ lacks the ‘a’ determinant region. FIG. 3B shows scanning electron microscopy images of HBsΔ⁹⁸ particles produced in the yeast Pichia pastoris. Bar, 100 nm. FIG. 3C depicts a Western blot of three self-assembling Hepatitis B Surface Antigen S domain proteins, showing monomers, dimers, trimers, and higher order multimers of: lane 1: CHep1HBsΔ¹⁵³ (SEQ ID: 10), lane 2: aCHep1HBsΔ⁹⁸ (SEQ ID NO: 7) and lane 3: CHep1HBsΔ⁹⁸ (SEQ ID NO: 5).

To aid expression of the membrane protein, certain DNA sequences encoding an HBsAg molecule, provided by the invention, may comprise codon-optimized DNA sequences, which contain silent DNA codon mutations. Silent DNA codon mutations encode the same amino acid as the wild-type codon, however with a different nucleotide sequence, exploiting the fact that multiple codons encode certain amino acids. Particularly the third position of any codon is usually variable and is referred to as the ‘wobble’ base. Codon-optimization is a process employing wobble base and other changes within the codons of the DNA to optimize the subsequent translation of a coding sequence, based on the different availability of tRNA molecules for certain amino acids in different species. SEQ ID NOs 18, 20, 22, 24, 26, 28, 30, 32, 34, 36, 38, 40, 42, 44, 46, 48, 50, 85, 87, 89, 91, 93, 95, 97, 99, 101, 103, 105, 107, 109, 111, 113, 115, 117 contain the DNA sequence of HBsAg molecules codon-optimized for yeast as examples of HBsAg molecule of the invention. SEQ ID NOs 19, 21, 23, 25, 27, 29, 31, 33, 35, 37, 39, 41, 43, 45, 47, 49, 51, 86, 88, 90, 92, 94, 96, 98, 100, 102, 104, 106, 108, 110, 112, 114, 116, 118 contain the DNA sequence of HBsAg molecules codon-optimized for mammalian cells as examples of HBsAg molecule of the invention.

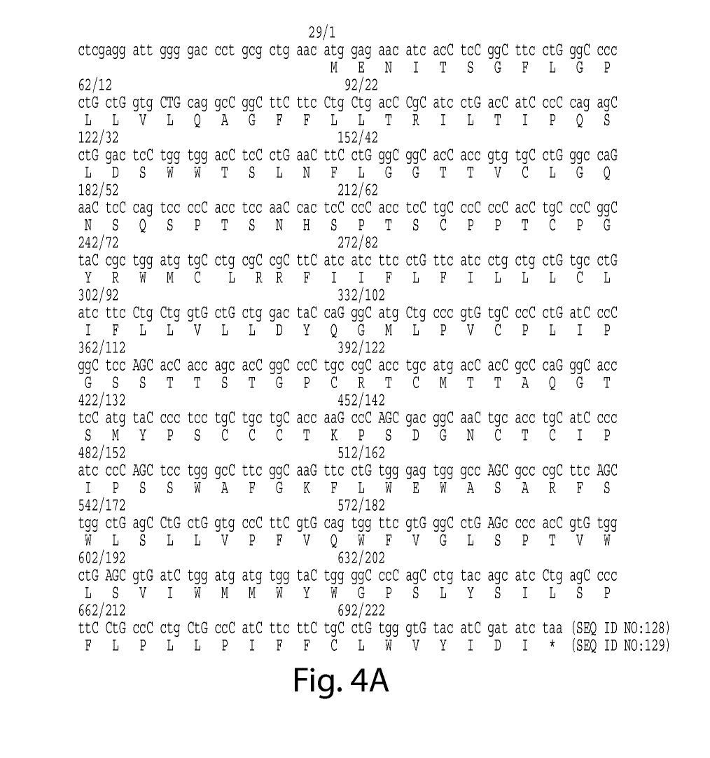

Non-limiting examples of codon-optimized and truncated HBsAg(S) proteins of the invention are provided in FIG. 4. FIG. 4A depicts the DNA and amino-acid sequence of full-length HBsAg(S) (SEQ ID NO: 128 and 129). The DNA sequence has been codon-optimized for expression in yeast (67% of codons were modified), introducing 155 silent mutation and several additional mutations in the amino acid sequence: L8F, K24R, P46T, A500, Q56P, I57T, S59N, C64S, I68T, C85F, K122R, T125M, P127T, F134Y, T143S, A159G, Y161F, V168A, N207S, I213L. The name of the wild-type amino acid is followed by the position of the amino acid in the sequence, which is followed by the name of the amino acid of the substitution/mutation introduced. The mutations are either within the ‘a’ determinant region, or outside the ‘a’ determinant region and can affect either immunogenicity or intra-molecular stability of the of the HBsAg(S) molecule, or both. FIG. 4B depicts the DNA and amino-acid sequence of HBsΔ⁹⁸FH (SEQ ID NO: 126 and 127), having a truncation of the two amino-terminal hydrophobic transmembrane domains (domains I and II, see FIG. 1B and FIG. 3A). The sequence contains the ‘a’ determinant region and incorporates the mutations outlined in FIG. 4A. Additionally, it features a Flag-Tag and a His-Tag at the C-terminus.

In some embodiments, expressed and/or isolated membrane proteins of the invention may be mixed in vitro with one or more natural and/or synthetic membrane components (for example, lipids or liposomes). It should be appreciated that nanoparticles of different average sizes or ranges of sizes may be produced.

In certain embodiments, non-natural amino acids are introduced into the HBsAg molecules, for example to enhance stability and/or bioavailability. Certain nanoparticles of the invention comprising HBsAg molecules comprising a deletion of one or more N-terminal transmembrane domains of the S domain have a reduced vesicle density to optimize delivery capabilities. Without wishing to be bound by theory, some nanoparticles are expected to have less structural rigidity, due to the limited number of transmembrane domains, than their natural counterparts, thereby facilitating substance loading and/or delivery. However, nanoparticles of the invention may retain a uniform structure that is useful for nanoscale fabrication and industrial scalability.

Accordingly, nanoparticles of the invention may have increased substance loading capacity. In some aspects, a nanoparticle with increased substance loading capacity as used herein is a nanoparticle that comprises a HBsAg protein comprising one or more deletions or mutations that increase, for example, the amount of substance that can be loaded relative to a nanoparticle comprising non-modified or wild-type HBsAg protein. The “amount” may be the number of molecules of the substance, such as agents or drugs, small molecules, proteins or nucleic acids, which can be loaded, and which may, for example, be measured optically (e.g., spectroscopically) or using any other suitable technique. An increased substance loading capacity may, for example, also refer to an increase in volume of the substance. As described herein, without wishing to be bound by theory, nanoparticles comprising HBsAg proteins lacking N-terminal domains and/or comprising additional mutations or deletions, may comprise membranes that are less dense (that is they are more fluid and are traversed more easily by substances residing outside the membrane-enclosed space of the nanoparticle) than nanoparticles comprising non-altered or wild-type HBsAg protein. In some embodiments, again without wishing to be bound by theory, membranes of the invention may allow for a greater density of substance packing in the nanoparticle relative to a particle comprising non-altered or wild-type HBsAg protein. Accordingly, in some embodiments, such nanoparticles comprising membranes that are less dense may have increased substance loading capacity. In some embodiments, nanoparticles comprising HBsAg proteins lacking N-terminal domains that are, for example, fused to other vesicles, such as liposomes, may have increased substance loading capacity.

Aspects of the invention relate to modified amino acid sequences relative to a normal or wild-type HBsAg sequence. It should be appreciated that different Hepatitis B virus sequences may be used as references for a normal or wild-type sequence. S genes of at least 568 human and 62 simian HBV strains are known. HBV strains may be distinguished by serological analysis and may be categorized into nine HBsAg subtypes designated ayw1, ayw2, ayw3, ayw4, ayr, adw2, adw4q−, adrq+, and adrq−. These correlate with eight genotypes (A-H), although several subtypes are encoded by more that one genotype. The genotypes (A-H) further comprise 19 subgenotypes (A1, A2, B1, B2, B3, B4, C1, C2, C3, C4, D1, D2, D3, D4, E, F1, F2, G, H). There are approximately three relatively highly conserved regions within the S gene product, for example, between residues 69 and 109, between residues 25 and 43 and 144 and 157. Between the conserved regions, there are several clusters of genotype-specific residues or variable sites. Most of the variable sites are found between residues 110 and 134 located within the exposed hydrophilic ‘a’-determinant region. The ‘a’ determinant region is the site of the majority of (B cell specific) HBV antigens. Major immune-escape mutations outside the ‘a’-determinant region are for example: Gly145Arg, Ile195Met, Trp196Leu, and Met197Ile. Four cysteines, at positions 124, 137, 139, and 149, are deemed important for the conformation of the tertiary structure of HBsAg and are conserved between strains. The region between Cys139 and Cys147, forming the second loop of the ‘a’-determinant is also conserved with two residues (Thr 140 and Thr 143) showing highly conserved genotype-specific substitutions (for example, Threonine to Serine substitutions).

For example, in some embodiments, recombinant or chimeric proteins of the invention may be based on a naturally occurring sequence of the HBsAg serotype ayw (e.g., the synthetic gene sequence having GenBank accession number AY515140.1). However, it should be appreciated that recombinant or chimeric proteins of the invention may be based on HBsAg proteins having different sequences, for example, sequences of different serotypes and/or genotypes and/or subgenotypes and/or immune-escape mutants, such as those described in Norder et al. Intervirology 47:289-309, 2004, which is hereby incorporated by reference in its entirety. Specifically incorporated are all sequences of all subgenotypes of S genes and of the pHBV3200 strain as presented in FIG. 4 of Norder et al.

Aspects of the invention are useful to deliver one or more substances to experimental or biological systems (for example, in vitro cell cultures, ex vivo cell or tissue preparations, in vivo organisms, etc.). Aspects of the invention may be useful to deliver one or more therapeutic, diagnostic, experimental, or other substances, or any combination thereof. In certain embodiments, the invention provides isolated hollow nanoparticles which may be used for delivery of a substance. In certain embodiments, the hollow nanoparticles may be used to transport a wide range of molecules including small molecules, nucleic acids and proteins, through the circulatory system of a subject and may be administered across mucosal barriers, orally, intra-venously, intra-peritoneally, topically, or via any other form of administration. Therapeutic or diagnostic agents can be transferred into the inner space of the hollow nanoparticle (or ‘loaded’) for example by simple diffusion-concentration mechanics, sonication, electroporation, or with the aid of an encapsulating double layer of self-assembling anionic or cationic lipids. However, agents may be incorporated into nanoparticles of the invention using any other suitable technique as aspects of the invention are not limited in this respect.

In some embodiments, chimeric proteins of the invention may include a fusion to one or more transmembrane domains that facilitate transport of nucleotides and/or to one or more amino acid sequences that bind receptors that are present specifically in or overexpressed on the surface of tumor cells.

According to the invention, a “hollow nanoparticle” comprises a membrane-enclosed vesicle, which can provide a luminal space, which is filled with a substance, which can be any gaseous, liquid, semi-solid or solid substance. A hollow nanoparticle may be spherical or tubular in shape, or could have any other shape. It should be appreciated that a hollow nanoparticle is not necessarily rigid and may take on different shapes. The membrane can consist of any material, for example, lipids, proteins, polysaccharides, other carbohydrates, synthetic or natural polymers. It should be appreciated that the membrane may consist of one or two or more of these materials. For example the membrane may comprise proteins, such as chimeric fusion proteins comprising one or more modified HBsAg(S) proteins and lipids of either natural origin (such as for example derived from a host cell or synthetic origin such as for example derived from a commercially produced liposome) or other synthetic or natural polymers. In some aspects, nanoparticles of the invention are based on transmembrane proteins that are adapted to form suitable vesicles that can be loaded with one or more substances for storage or delivery. Certain nanoparticles of the invention comprise a Hepatitis B Surface Antigen S protein domain, HBsAg(S). In some embodiments, the only HBsAg proteins in a nanoparticle of the invention are HBsAg(S) variants that lack one or both of their N-terminal transmembrane domains (domains I and II as described herein) or portions thereof, and optionally also lack the ‘a’ region or a portion thereof. However, in some embodiments, vesicle membranes may contain more than one type of protein. Other unrelated proteins also may be included (for example, for targeting, enzymatic activity, receptor binding, ligand binding, etc., or any combination thereof). The membrane of certain nanoparticles may comprise a Hepatitis B Surface Antigen S domain protein, and in addition any other lipid, protein, polysaccharide, other carbohydrates, synthetic or natural polymers. The membrane, in certain embodiments, may, for example comprise a Hepatitis B Surface Antigen S domain protein, lipids, which may be derived from the endoplasmic reticulum or may be synthetic or organic from another source, and in addition the membrane may comprise an antibody, a targeting molecule, or a receptor molecule. The membrane may, for example, be fully permeable or semi-permeable or not permeable. The outer and inner membrane surfaces may be hydrophobic or hydrophilic, or one surface may be hydrophobic and one hydrophilic, and the particle may behave like a micelle.

The combination of membrane material and protein forms a membrane-enclosed vesicle that can be loaded with one or more substances for storage and/or delivery applications. It should be appreciated that in some embodiments the membrane of the nanoparticle (membrane-enclosed vesicle) comprises at least one protein, such as a chimeric fusion protein, for example comprising HBsAg(S) proteins lacking at least one N-terminal transmembrane domain or a portion thereof as described herein, and optionally additional membrane material. In some embodiments, vesicles of the invention are referred to as “nanoparticles” and have a diameter of between about 1 nm and about 1,000 nm (for example, about less than 50 nm (5-50 nm, 10-40 nm, 15-30 nm, 20-30 nm, 25-35 nm, 25-30 nm, 27-30 nm), about 50-100 nm, about 100-150 nm, about 150-200 nm, about 200-250 nm, about 250-500 nm, about 500-1,000, about 5 nm, about 10 nm, about 15 nm, about 20 nm, about 25 nm, about 30 nm, about 35 nm, about 40 nm, about 45 nm, about 50 nm, or any other intermediate size). Certain nanoparticles of the invention are sufficiently small (for example, approximately 25 nm in diameter) to efficiently mediate transcytosis through cell layers or barriers (for example epithelial call layers) and/or cell entry (for example through receptor-mediated endocytosis). Certain nanoparticles of 20-30 nm are optimal for drug delivery (Gao H et al., PNAS 102(27):9469-74, 2005). It should be appreciated that the diameter of a vesicle may refer to the average diameter of a vesicle in a preparation of vesicles that contains vesicles of different diameters. However, in some embodiments, a vesicle preparation is relatively homogeneous and contains vesicles having a narrow range of vesicle diameters. It should be appreciated that although the vesicles are described as having a diameter as if they were perfectly spherical (see, for example, FIG. 3B), they are in fact non-rigid structures that may have a generally spherical shape but also may have other geometric shapes (for example, elongated, filamentous, irregular, etc.) depending on the protein and membrane material content of the vesicle membrane (for example, the relative amount or numbers of molecules of protein and membrane material in each particle), the substance(s) within the vesicle, the environment of the vesicle, temperature, and other factors.

It should be appreciated that the size of the nanoparticle may be influenced by a number of parameters, such as for example the size and number of the modified HBsAg(S) protein monomers or chimeric fusion proteins thereof forming the nanoparticle, and the absence or presence of additional membrane proteins or lipids. Nanoparticles comprising modified HBsAg(S) protein domains of the invention may be fused to liposomes, resulting in a considerably larger nanoparticle (for example 50-500 nm). Alternatively, nanoparticles may be treated with chemical agents, such as detergents, to strip or remove lipids out of the nanoparticle, therefore reducing the size of the nanoparticles (for example, less than 50 nm: 5-50 nm, 10-40 nm, 15-30 nm, 20-30 nm, 25-35 nm, 25-30 nm, 27-30 nm).

Nanoparticles of the invention may have between 5 and 1,000 (or more, for example, 5,000 or more) copies of a variant HBsAg (for example, variant HBsAg(S)) protein of the invention per nanoparticle. For example, an average nanoparticle may have about 10, about 25, about 50, about 75, about 100, about 125, about 150, about 175, about 200, about 250, or more molecules of the variant transmembrane domain.

Nanoparticles may be loaded with one or more substances. They may be loaded by electroporation; sonication; expression of the vesicle protein along with one or more soluble proteins, or in the presence of one or more substances, that are packaged in the vesicle; fusion with liposomes (e.g., containing one or more substances), in vitro assembly with one or more substances, diffusion or any other suitable technique, or any combination thereof, as the invention is not limited in this respect. Accordingly, it should be appreciated that a nanoparticle of the invention may be loaded with a single type of substance (for example, many copies of a unique substance) or two or more (for example, 2, 3, 4, 5, 6, 7, 8, 9, 10, etc.) different substances.

As used herein a “substance” is any gaseous, liquid, semi-solid or solid substance that would be suitable for loading into the HBsAg(S) nanoparticles. Preferably, the substance will have an effect on a subject, for example, a diagnostic, prophylactic or therapeutic effect. The substance, may be, for example, a cellular component or any other active agent, for example, a drug or a gene vector capable of expressing a peptide, a small molecule, etc. The substance is one that is added to the HBsAg(S) nanoparticles. Examples of the substance are gene delivery vehicles (plasmids, viral and non-viral vectors), additional cellular components, genetically engineered or naïve, recombinant, soluble or any other type of proteins, peptides, cytokines or other signaling molecules, which can have pro- or anti-inflammatory effects, or pro- or anti-anti-apoptotic effects, polysaccharides, glycoproteins, heterogeneous mixtures of macromolecules (for example, a natural product extract) and hybrid macromolecules (for example, protein/nucleic acid hybrids, albumin conjugated proteins, drugs, inorganic molecules, organic molecules, or combinations thereof), or other bioactive molecules, such as growth factors, such as, for example members of the transforming growth factor-β (TGF-β) super family, bone morphogenetic proteins (BMPs), fibroblast growth factors, growth hormone, and insulin-like growth factors (IGFs), antibodies, other nucleic acids (for example, RNA, DNA, PNA, multiplexes of them (for example, triplex)), preferably siRNA and antisense RNA, and/or cytotoxic drugs. In some embodiments, diagnostic, prophylactic or therapeutic substances used according to aspects of the invention are sterile.

In certain embodiments, nanoparticles comprising HBsAg molecules comprising a deletion of one or more N-terminal transmembrane domains of the S domain having a small overall size, for example less than 50 nm (for example 5-50 nm, 10-40 nm, 15-30 nm, 20-30 nm, 25-35 nm, 25-30 nm, 27-30 nm), may be particularly useful for loading large DNA molecules that have a propensity to aggregate on the outer surface of larger vesicles.

A “therapeutic substance” is any substance that has a therapeutic effect on a subject and may be, for example a chemotherapeutic or immunotherapeutic substance.

A chemotherapeutic agent may be, for instance, methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Ince/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARD inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) or Vindesine sulfate, but it is not so limited.

An immunotherapeutic agent may be, for instance, Ributaxin, Herceptin, Quadramet, Panorex, IDEC-Y2B8, BEC2, C225, Oncolym, SMART M195, ATRAGEN, Ovarex, Bexxar, LDP-03, for t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, for egf.r3, for c5, BABS, anti-FLK-2, MDX-260, ANA Ab, SMART 1D10 Ab, SMART ABL 364 Ab or ImmuRAIT-CEA, but it is not so limited.

A therapeutic substance may also be any of the following agents: adrenergic agent; adrenocortical steroid; adrenocortical suppressant; agents for treating cognition, antiplatelets, aldosterone antagonist; amino acid; anabolic; analeptic; analgesic; anesthetic; anorectic; anti-acne agent; anti-adrenergic; anti-allergic; anti-Alzheimer's, anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; anxiolytics, appetite suppressant; blood glucose regulator; bone resorption inhibitor; bronchodilator; cardiovascular agent; cholinergic; COX1 inhibitors, COX2 inhibitors, direct thrombin inhibitors, depressant; diagnostic aid; diuretic; dopaminergic agent; estrogen receptor agonist; fibrinolytic; fluorescent agent; free oxygen radical scavenger; gastrointestinal motility effector; glucocorticoid; GPIIbIIIa antagonists, hair growth stimulant; hemostatic; histamine H2 receptor antagonists; hormone; human growth hormone, hypocholesterolemic; hypoglycemic; hypolipidemic; hypnotics, hypotensive; imaging agent; immunological agents such as immunizing agents, immunomodulators, immunoregulators, immunostimulants, and immunosuppressants; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; proton pump inhibitors, psychotropic; radioactive agent; scabicide; sclerosing agent; sedative; sedative-hypnotic; selective adenosine Al antagonist; serotonin antagonist; serotonin inhibitor; serotonin receptor antagonist; statins, steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor, but it is not so limited.

A “diagnostic substance” is any substance that has diagnostic capabilities, for example imaging agents, such as detectable markers, for example heavy metals, Gadolinium, Quantum dots, magnetic particle, radioactive particles, labeled antibodies, luciferase and other chemoluminescent agents. These agents may be substances inside the hollow nanoparticle, and/or on the surface (for example, outer-surface) of the membrane. These agents may be used to detect an adverse condition by any medical detection device or method, such as for example Magnetic Resonance Imaging (MRI), Positron Emission Tomography (PET), Computerized Axial Tomography (CAT), X-rays, or other imaging modalities. These applications may provide for immediate monitoring and/or diagnosis of early metastasis. It should be appreciated that these imaging embodiments may be combined with delivery embodiments, for example, to allow for simultaneous treatment and monitoring (for example, to confirm that treatment is appropriately localized to a target site such as a tumor or other diseased tissue).

The HBsAg nanoparticles of the invention can, in some embodiments, comprise “gene delivery vehicles”, such as viral and non-viral vectors comprising a therapeutically useful gene. As used herein, a “gene” is an isolated nucleic acid molecule of greater than thirty nucleotides, more typically one hundred nucleotides or more, in length. It generally will be under the control of an appropriate promoter, which may be inducible, repressible, or constitutive. Any genes that would be useful in replacing or supplementing a desired function, or achieving a desired effect such as the inhibition of tumor growth, could be introduced using the HBsAg nanoparticles described herein. Promoters can be general promoters, yielding expression in a variety of mammalian cells, or cell specific, or even nuclear versus cytoplasmic specific. These are known to those skilled in the art and can be constructed using standard molecular biology protocols. Any type of gene is useful according to the methods of the invention. The specific genes used in a particular circumstance will depend on the condition being treated and/or the desired therapeutic result. By delivering the cDNAs that code for proteins with reparative or therapeutic potential to specific cells at sites of injury or disease, the genetically-modified cells become local factors for drug production, permitting sustained synthesis of the specific protein. Suitable promoters, enhancers, vectors, etc., for such genes are published in the literature. In general, useful genes replace or supplement function, including genes encoding missing enzymes. Genes which affect regulation can also be administered, alone or in combination with a gene supplementing or replacing a specific function. For example, a gene encoding a protein which suppresses expression of a particular protein-encoding gene can be administered by the HBsAg nanoparticles of the invention. Genes can be obtained or derived from a variety of sources, literature references, Genbank, or commercial suppliers. They can be synthesized using solid phase synthesis if relatively small, obtained from deposited samples such as those deposited with the American Type Culture Collection, Rockville, Md. or isolated de novo using published sequence information.

In addition to genes, the substance may be a short oligonucleotides such as antisense and ribozymes which are distinguished from genes by their length and function. Unlike such short oligonucleotides, genes encode protein and therefore will typically be a minimum of greater than 100 base pairs in length, more typically in the hundreds of base pairs.

In some aspects of the invention, nucleic acids that may be encapsulated in the nanoparticles comprising HBsAg proteins described herein can be DNA and/or RNA molecules. In some aspects, the invention relates to the use of small nucleic acid molecules, including antisense nucleic acids and short interfering nucleic acid (siNA), the latter include, for example: microRNA (miRNA), short interfering RNA (siRNA), double-stranded RNA (dsRNA), and short hairpin RNA (shRNA) molecules to knockdown expression of target genes associated with a disease or disorder. The siNA can be unmodified or chemically-modified. The siNA can be chemically synthesized (for example as a short oligonucleotide), expressed from an expression vector (for example linked to a promoter element) or enzymatically synthesized. Short oligonucleotides may, for example, be chemically-modified synthetic short interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules). In some embodiments, modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al, International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., molecule comprises one or more chemical modifications.

In some embodiments, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase H or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In certain embodiments transgenes and expression vectors are controlled by inducible promoters, such as tetracycline inducible expression systems. Such plasmids and/or expression vectors may be loaded into the nanoparticles described herein.

In some embodiments, a small interfering nucleic acid of the invention is expressed in mammalian cells using a mammalian expression vector. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters.

siRNA molecules are well know in the art and many siRNAs are known that target tumor-specific proteins that may be mutated, overexpressed and/or deregulated.

Accordingly, aspects of the invention can be used to deliver molecules that promote RNA interference using any of a variety of molecules known in the art, e.g., short interfering RNA molecules (siRNA), which are double stranded RNA molecules. As described herein, RNA interference (RNAi) is a phenomenon describing double-stranded (ds)RNA-dependent gene specific posttranscriptional silencing. Synthetic duplexes of 21 nucleotide RNAs can mediate gene specific RNAi in mammalian cells, without invoking generic antiviral defense mechanisms (Elbashir et al. Nature 2001, 411:494-498; Caplen et al. Proc Natl Acad Sci 2001, 98:9742-9747).

In some embodiments, polynucleotides are provided comprising an RNAi sequence that acts through an RNAi mechanism to attenuate or inhibit expression of a gene of interest, e.g., a gene that is overexpressed in cancer. In some embodiments, the siRNA sequence is between about 19 nucleotides and about 75 nucleotides in length, or between about 25 base pairs and about 35 base pairs in length. An RNAi construct contains a nucleotide sequence that hybridizes under physiologic conditions of the cell to the nucleotide sequence of at least a portion of the mRNA transcript of a gene of interest. In certain embodiments, the double-stranded RNA need only be sufficiently similar to natural RNA that it has the ability to mediate RNAi. In certain embodiments, the number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

In certain embodiments, sequence identity may be optimized by sequence comparison and alignment algorithms known in the art (see Gribskov and Devereux, Sequence Analysis Primer, Stockton Press, 1991) and calculating the percent difference between the nucleotide sequences by, for example, the Smith-Waterman algorithm as implemented in the BESTFIT software program using default parameters (e.g., University of Wisconsin Genetic Computing Group). In certain embodiments, the sequence identity between the inhibitory RNA and the portion of the target gene is greater than 85%, 86%, 87%, 88%, 89%, 90%, 91%, 92%, 93%, 94%, 95%, 96%, 97%, 98%, 99%, or is 100%.

Production of polynucleotides comprising RNAi sequences is well known in the art. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. In certain embodiments, the polynucleotides that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. In certain embodiments, the siRNA polynucleotides may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxyl group.

The siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA molecules. In addition, chromatography (e.g., size exclusion chromatography), glycerol gradient centrifugation, affinity purification with antibody can be used to purify siRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

Tools for design and quality of siRNAs, shRNAs and/or miRNAs are known in the art Web-based online software system for designing siRNA sequences and scrambled siRNA sequences are for example siDirect, siSearch, SEQ2SVM, Deqor, siRNA Wizard (InvivoGen). The specificity can be predicted using for example SpecificityServer, miRacle. Target sequences can be researched for example at HuSiDa (Human siRNA Database), and siRNAdb (a database of siRNA sequences).

Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190). Such antisense molecules may also be loaded into the nanoparticles described herein.

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases suppression strategies have led to a reduction in RNA levels without a concomitant reduction in proteins, whereas in others, reductions in RNA have been mirrored by reductions in protein. Such triple helix molecules may also be loaded into the nanoparticles described herein.

It should be appreciated that isolated or purified RNA molecules, e.g., siRNA molecules or other small RNA molecules may be packaged into a nanoparticle of the invention for delivery. However, it also should be appreciated that in some embodiments, a nucleic acid (e.g., a DNA molecule) that expresses an RNA molecule, e.g., an siRNA or other small RNA molecule, may be packaged into a nanoparticle of the invention. Accordingly, the RNA-expressing nucleic acid may be delivered by the nanoparticle and the RNA is subsequently expressed after delivery (e.g., in a target tissue or cell at the delivery site).

In some embodiments, a vector encoding a therapeutic RNA and/or protein of the invention may be packaged into a nanoparticle. As used herein, vectors are agents that transport the gene into a cell without degradation and include a promoter yielding expression of the gene in the cells into which it is delivered. In some embodiments, a virus vector (for example, a virus genome or portion thereof, optionally packaged in a viral particle or portion thereof) for delivering a gene may be used, for example, selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (for example, Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Viral. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci, USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 76:1951-1959, 1996). Any of these viral vectors that have previously been delivered in viral particles may be packaged and delivered in a nanoparticle of the invention. In some embodiments, the virus vector is an adenovirus or an alphavirus vector and is delivered in a nanoparticle of the invention.

Another useful virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. In some embodiments, a viral vector based on an adeno-associated viral DNA sequence may be packaged and delivered using a nanoparticle of the invention.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (e.g., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991). Aspects of the invention also may be used to package or deliver any one of these viral vectors.

Accordingly, it should be appreciated that any of the nucleic acids described herein that encode and/or express therapeutic or other nucleic acids or proteins of interest may be used as gene-delivery vehicles and packaged in nanoparticles of the invention.

In some aspects, methods and compositions of the invention may be used along with other virus-based particles as described herein (e.g., to make particles for delivery of substance to subjects to treat diseases such as cancer, or for any other application described herein).

As used herein, “tumor” and “cancer” are being used interchangeably. The cancer or tumor may be malignant or non-malignant. Cancers or tumors include but are not limited to biliary tract cancer; bladder cancer, brain cancer; breast cancer; cervical cancer; choriocarcinoma; colon cancer; endometrial cancer; esophageal cancer; gastric cancer; intraepithelial neoplasms; lymphomas; liver cancer; lung cancer (for example, small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma. Aspects of the invention may be used to deliver agents or drugs to cancerous cells, tissues, or organs.

The term “effective amount” of a composition refers to the amount necessary or sufficient for a composition alone, or together with further doses, to realize a desired biologic effect. The desired response, of course, will depend on the particular condition being treated. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or adverse condition being treated, the size of the subject, or the severity of the disease or adverse condition. In some embodiments, a maximum dose of the individual components or combinations thereof may be used, that is, the highest safe dose according to sound medical judgment. It will be understood by those of ordinary skill in the art, however, that a patient may insist upon a lower dose or tolerable dose for medical reasons, psychological reasons or for virtually any other reasons. One of ordinary skill in the art can empirically determine the effective amount without necessitating undue experimentation.

For any compound or composition described herein the therapeutically effective amount can be initially determined from animal models. A therapeutically effective dose can also be determined from data for compounds or compositions which are known to exhibit similar pharmacological activities, such as other nanoparticles. The applied dose can be adjusted based on the relative bioavailability and potency of the administered compound or composition. Adjusting the dose to achieve maximal efficacy based on the methods described above and other methods as are well-known in the art is well within the capabilities of the ordinarily skilled artisan.

As used herein, the terms “treat,” “treated,” or “treating” when used with respect to an adverse condition, such as a disorder or disease, for example, an infectious disease, cancer, allergy, or asthma refers either to a prophylactic treatment which increases the resistance of a subject to development of the adverse condition, or, in other words, decreases the likelihood that the subject will develop the adverse condition, or to a treatment after the subject has developed the adverse condition in order to fight the disease, or prevent the adverse condition from becoming worse, or to any combination thereof.

A “subject” shall mean a human or vertebrate animal or mammal including but not limited to a dog, cat, horse, cow, pig, sheep, goat, turkey, chicken, primate, for example, monkey, and fish. Thus, the compounds may be used to treat cancer and tumors, infections, and allergy/asthma in human and non-human subjects.

The HBsAg nanoparticles may be administered per se (neat) or in the form of a pharmaceutically acceptable composition or solution (e.g., a pharmaceutically acceptable salt). If the formulations of the invention are administered in pharmaceutically acceptable solutions, they may routinely contain pharmaceutically acceptable concentrations of salt, buffering agents, preservatives, compatible carriers, adjuvants, and optionally other therapeutic ingredients. The solutions used preferably are sterile.

When used in medicine, the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to: acetic acid and a salt (1-2% w/v); citric acid and a salt (1-3% w/v); boric acid and a salt (0.5-2.5% w/v); and phosphoric acid and a salt (0.8-2% w/v). Suitable preservatives include, but are not limited to: benzalkonium chloride (0.003-0.03% w/v); chlorobutanol (0.3-0.9% w/v); parabens (0.01-0.25% w/v); and thimerosal (0.004-0.02% w/v). Accordingly, pharmaceutical compositions of the invention can contain an effective amount of HBsAg nanoparticles optionally included in a pharmaceutically-acceptable carrier.

In some embodiments, nanoparticles of the invention may be formulated for oral, intra-venous, intra-peritoneal, topical, or other form of delivery. For oral delivery nanoparticles of the invention may be formulated in capsules or other devices to pass through the stomach. In some embodiments, a nanoparticle comprising an HBsAg(S) protein lacking an “a” region and/or one or more amino-terminal sequences but having a specific targeting agent (for example, antibody) may be used for oral delivery. In certain embodiments, a nanoparticle comprising an HBsAg(S) protein may further comprise an albumin domain peptide and may be used for oral delivery to enhance resorption through the gut. In certain embodiments, nanoparticles comprising an HBsAg(S) protein described herein that are formulated for oral delivery are stable and have high bioavailability. In certain embodiments, nanoparticles formulated for oral delivery may be highly pH resistant and/or highly resistant to proteinase activity thereby increasing stability in the stomach and/or gut.

Aspects of the invention (for example, deletions or mutations of one or two N-terminal transmembrane domains to prepare protein variants to be incorporated into nanoparticle membranes, for example, for use as substance delivery vehicles) may also apply to other surface antigens (sAgs), for example, other viral surface antigens, with high amino acid sequence similarity to HBsAg, for example having 99%, 95%, 90%, 85%, 80%, 75% amino acid sequence identity, particularly in the transmembrane domains.

Aspects of the invention are not limited in its application to the details of construction and the arrangement of components set forth in the preceding description or illustrated in the examples or in the drawings. Aspects of the invention are capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

The following examples are non-limiting. Methods and compositions illustrated in the examples may be applied to or combined with other embodiments or aspects of the invention as described herein.

### Example 1

### Codon Optimization of Genes for Enhanced Human and Yeast Expression

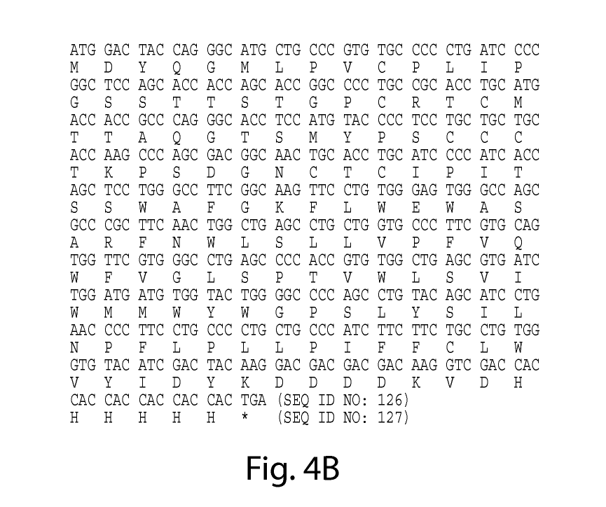

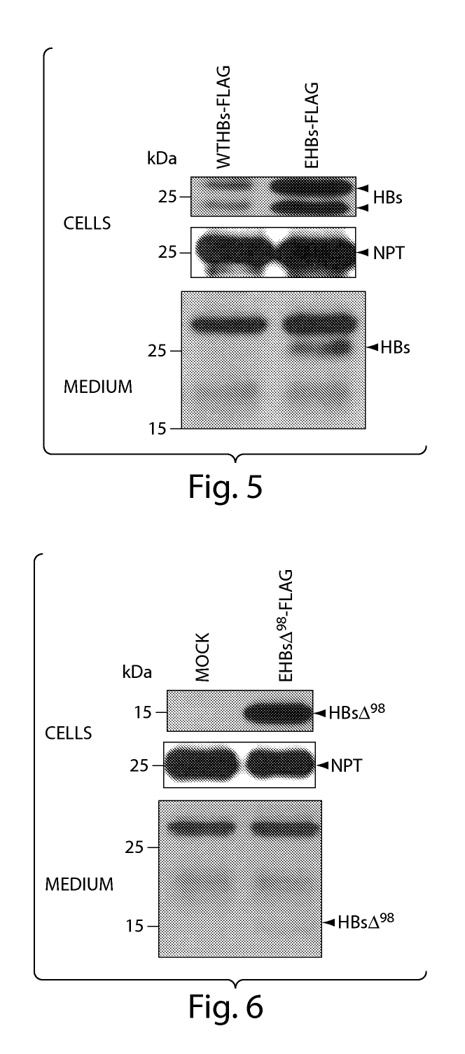

Codon optimization of genes for enhanced expression of the proteins of this invention in human cells was performed by assembling synthetic sequences containing codons chosen among those preferred by highly expressed human genes, following algorithms described previously (Cid-Arregui et al J. Virol. 77:4928-4937, 2003), as it was done for the complete HBsAg sequence (Genbank accession number AY515140). For optimization of codons for enhanced expression in yeast, an algorithm was elaborated on the basis of the relative synonymous codon usage found in highly expressed genes from yeast as described by Sharp et al. Nucl. Acids Res. 14: 5125-5143 (1986). The sequences encoding the different parts of the fusion genes were assembled in frame and verified by sequencing. The superior expression driven by the synthetic genes was demonstrated by transfection experiments in which wild type and codon-optimized genes were compared, as shown in FIG. 5 and FIG. 6 with human cells.

### Example 2

### Expression and Purification of HBsAg Particles in Recombinant Yeast

(1) The recombinant yeast (Saccharomyces cerevisiae YPH 499 strain) transformed with pESC-URA (Stratagene) carrying codon-optimized fusion genes (SEQ ID No. 22, 24, 26, 28, 30, 32, 34, 36, 38) were cultivated following manufacturer's recommendations.

(2) From the recombinant yeast in the stationary growth phase (after about 72 hours), whole cell extracts were prepared using Yeast Protein Extraction Reagent (Pierce Chemical Co.), then separated by sodium dodecylsulfate-polyacrylamide gel electrophoresis (SDS-PAGE), and subjected to silver staining to identify HBsAg in the sample.

(3) The recombinant yeast (wet weight: 26 g) cultivated on the synthetic medium 8S5N-P400 was suspended in 100 ml of Buffer Solution A (7.5 M urea, 0.1 M sodium phosphate, pH 7.2, 15 mM EDTA, 2 mM PMSF, 0.1% Tween 80) and homogenized in a BEAD-BEATER (BioSpec Products, Inc.) using 0.5 mm glass beads. Then, the supernatant was recovered by centrifugation. Subsequently, the supernatant was mixed with 0.75-fold volume of 33% (w/w) PEG 6000, and cooled on ice for 30 minutes. Then, the mixture was centrifuged (7,000 rpm, 30 minutes) to recover pellets. The pellets were then re-suspended in Buffer Solution A without Tween 80.

The re-suspended solution was layered over a CsCl solution of 10-40% gradient and subjected to ultracentrifugation at 28,000 rpm for 16 hours. After centrifugation, the sample was divided into 12 fractions, which were subjected to Western Blotting (primary antibody was anti-HBsAg monoclonal antibody) to identify the fraction containing HBsAg. Further, the fraction containing HBsAg was dialyzed in Buffer Solution A without Tween 80.

(4) The dialysate (12 ml) obtained in (3) was layered over a sucrose of 5-50% gradient and subjected to ultracentrifugation at 28,000 rpm for 16 hours. In the same manner as in (3), the fraction containing HBsAg after centrifugation was identified and dialyzed in Buffer A containing 0.85% NaCl instead of urea and Tween 80. ((2) to (4)).

(5) The procedure of (4) was repeated and the sample after dialysis was concentrated using Ultra Filter Q2000 (Advantech Co.) and refrigerated at 4 degrees Celsius until use. From the result of Western Blotting (3) after CsCl density equilibrium centrifugation, The fusion protein was found to have a molecular weight of 28 kDa. A total of about 20 mg of purified HBsAg particles were obtained from 26 g (wet weight) of the fungus body derived from 2.5 L of culture medium.

The fractions obtained in the course of purification were analyzed by silver or coomassie staining after SDS-PAGE. Further, to confirm that protease derived from yeast was removed by purification, the purified particles obtained in (5) were incubated at 37° C. for 12 hours, then subjected to SDS-PAGE, and identified by silver staining. Finally, it was confirmed that protease derived from yeast was completely removed by the overall purification process. Purified fractions were collected, desalted and concentrated using AMICON concentrators (Millipore) with 10 kDa cut-off filters. The samples were washed 4 times each with 5 ml of sterile deionized water and processed for visualization of nanoparticles by electron microscopy after negative staining with uranile acetate (FIG. 3B).

### Example 3

### Preparation of Nanoparticles Displaying a Hepatocyte Targeting Moiety (Hep-Nanoparticles)

Hep nanoparticles like those listed in Table 1 (SEQ ID No. 3-11) contain a Pre-S1-derived sequence that has been shown to bind a cell membrane receptor on hepatic cells and to facilitate hepatitis B virus internalization (De Falco et al. J. Biol. Chem. 276:36613-36623, 2001). Codon-optimized genes encoding these fusion proteins (SEQ ID No. 22, 24, 26, 28, 30, 32, 34, 36, 38) were cloned under control of the PAOX1 promoter, which is induced by methanol, in the polylinker of the plasmid pPICZA (Invitrogen) in frame with a HIS-tag and used form transformation of the yeast Pichia pastoris using the EASYSELECT Pichia expression kit (Invitrogen) following manufacturer's instructions. Yeast cells were transformed by electroporation as follows: Pichia pastoris X-33 was grown in 5 ml YPD medium overnight at 30° C. 0.1-0.5 ml of the overnight culture were used to inoculate 500 ml of fresh medium in a 2 liter flask and incubated until OD₆₀₀=1.3-1.5. Then the cells were centrifuged at 1500×g for 5 minutes at +4° C., washed once with ice-cold sterile water, once with ice-cold 1 M sorbitol and the pellet re-suspended in 1 ml of ice-cold 1 M sorbitol for a final volume of approximately 1.5 ml. For electroporation, aliquots of 80 μl of the cells were mixed with 10 μg of linearized plasmid, transferred to 0.2 cm electroporation cuvettes and pulsed in a MULTIPORATOR (Eppendorf) according to the manufacturer's instructions for yeast. Immediately after electroporation 1 ml of ice-cold 1 M sorbitol was added to the cuvette and the cells were incubated at 30° C. without shaking for 1.5 hours. Finally, the cells were plated on separate, labeled YPDS plates containing 1000 μg/ml Zeocin™ for selection of multi-copy recombinants.

For protein expression, a single colony was inoculated in 25 ml of buffered complex medium containing glycerol (BMGY: 1% yeast extract, 2% peptone, 100 Mm potassium phosphate, pH 6.0, 1.34% yeast nitrogen base, 4×10⁻⁵ biotin, 1% glycerol) in a 1 liter flask, grown at 28-30° C. in a shaking incubator (250-300 rpm) until the culture reaches an OD₆₀₀=2-6. Then the cells were harvested and re-suspended to an OD₆₀₀ of 1.0 in BMMY medium (similar to BMMG, but with 0.5% methanol, instead of 1% glycerol) to induce expression. Methanol was added every 24 hours to a final concentration of 0.5% to maintain induction for 5 days. Then, cell pellets were quick frozen in a dry ice/alcohol bath and kept at −80° C. until needed.

Protein extracts from cell pellets were prepared under non-denaturing conditions by re-suspending the cells in lysis buffer (50 mM NaH₂PO₄, 300 mM NaCl, 10 mM Imidazole, 0.05% Tween 20, pH 8.0) supplemented with a protease inhibitor cocktail (COMPLETE, Roche) and disrupting them in a BEADBEATER (BioSpec Products, Inc.) using 0.5 mm glass beads operated with two pulses of 1.5 minutes each separated by an interval of 1 minute to maintain temperature below 30° C. The lysates were centrifuged at 15000×g for 30 minutes and the supernatants were processed for IMAC (ion metal affinity chromatography) using Ni-NTA agarose columns. Extensive washing to remove nonspecifically bound proteins was performed with Wash buffer (50 mM NaH₂PO₄, 300 mM NaCl, 20 mM Imidazole, 0.05% Tween 20, pH 8.0). Finally, bound proteins were eluted with Elution buffer (50 mM NaH₂PO₄, 300 mM NaCl, 250 mM Imidazole, 0.05% Tween 20, pH 8.0). Elution fractions were analyzed by SDS-PAGE and Western blotting (FIG. 3C). Elution fractions were then collected, desalted and concentrated using AMICON concentrators (Millipore) with 10 kDa cut-off filters. The samples were washed 4 times each with 5 ml of either sterile deionized water or hypo-osmolar (25 mM KCl, 0.3 mM KH₂PO₄, 0.85 mM K₂HPO₄, myo-Inositol to 90 mOsmol/kg, pH 7.2, conductivity at 25° C. 3.5 mS/cm±10%) or iso-osmolar (25 mM KCl, 0.3 mM KH₂PO₄, 0.85 mM K₂HPO₄, myo-Inositol to 280 mOsmol/kg, pH 7.2, conductivity at 25° C. 3.5 mS/cm±10%) buffers (Eppendorf AG, Hamburg). Protein concentration was determined by BCA (Pierce).

### Example 4

### Expression and Secretion to the Medium of Wild Type HBsAg(S) and Mutant HBsΔ⁹⁸ in a Human Cell-Line

Human HEK 293 cells were transfected with a plasmid encoding a bicistronic mRNA containing an internal ribosomal entry site (IRES) sequence from a cytomegalovirus (CMV) promoter. The 5′-end of the transcript encodes HBsAg, the sequence downstream the IRES encodes neomycin phosphotransferase (NPT), which serves here as an internal control. After transfection the cells were incubated 48 hours at 37° C./5% CO₂. Then, the medium was collected and used for immunoprecipitation and the cells lysed in Laemmli sample buffer. Immunoprecipitations were performed with anti-Flag antibodies. A) Comparison of expression of wild type (WTHBs-Flag) and codon-optimized (EHBs-Flag) genes encoding full length HBs. The amount of HBs protein was estimated 20-fold higher with the codon-optimized gene, while the levels of NPT protein were nearly equal (see, FIG. 5). B) Cells were transfected with plasmid carrying a codon-optimized HBsΔ⁹⁸ (EHBsΔ⁹⁸-Flag). The HBsΔ⁹⁸ protein has a molecular weight of about 15 kDa, which in spite of the N-terminal 98 amino acid deletion is secreted to the medium. Mock: cells transfected with empty plasmid (see, FIG. 6).

FIG. 5 shows a Western blot of HEK 293 cells transfected with plasmid pIRES-neo2-HBsAgWT carrying a wild-type HBsAg gene encoding wild-type HBsAg protein with a Flag-tag (WTHBs-Flag, SEQ ID NO: 119 and 120), or with plasmid pIRES-neo2-EHBsAg carrying a synthetic HBsAg gene, codon-optimized for expression in human cells, encoding also wild-type HBsAg protein with a Flag-tag (EHBsAg-Flag, SEQ ID NOs: 121 and 122). The sequence for pIRES-neo2-plasmid is SEQ ID No: 123. The upper panels show a total cell extract, with NPT protein as a loading control. The lower panel shows secreted protein immunoprecipitated from the culture medium. Immunoprecipitation and detection was with anti-Flag antibodies.

FIG. 6 shows a Western blot of HEK 293 cells transfected with plasmid pIRES-neo2-EHBsΔ⁹⁸ carrying a mutant HBsAg gene, codon-optimized for expression in human cells, which encodes a truncated form of HBsAg protein with a Flag-tag (HBsΔ⁹⁸FH, SEQ ID NO: 57), or transfected with plasmid pIRES-neo2 (SEQ ID NO: 123) without insert as control. The upper panels show a total cell extract, with NPT protein as a loading control. The lower panel shows secreted protein immunoprecipitated from the culture medium. Immunoprecipitation and detection was with anti-Flag antibodies.

### Isolation of Proteins of the Invention:

Cells growing on 6 cm plates were transfected with 2 μg of plasmid as indicated and incubated for 48 hours. The cells were then washed twice with phosphate buffered saline (PBS), and lysed in SDS loading buffer containing 1 mM DTT. The cellular proteins were separated on 15% polyacrylamide gels by SDS-PAGE, blotted onto PVDF membranes, blocked with 5% milk in PBS containing 0.1% Tween 20 and incubated at room temperature with HRP-conjugated anti-FLAG M2 antibodies (Sigma). Antibody binding was visualized with enhanced chemiluminescence reagent (Renaissance®, NEN-Perkin Elmer). For immunoprecipitation, after 48-72 hrs transfected cells were. lysed and HBsAg-FLAG protein was immunoprecipitated using the FLAG Immunoprecipitation Kit according to the manufactures instructions (Sigma-Aldrich) or using anti-FLAG antibody M2 (Sigma-Aldrich). Bound proteins were washed, eluted, and detected by Western blotting.

### Example 5

### Cytotoxicity Assay with the Cytotoxic Drug Gemcitabine Gemcitabine Encapsulated into Nanoparticles

Gemcitabine was encapsulated into HBsΔ⁹⁸ as follows: a purified suspension of nanoparticles (100 μg) in deionized water or iso-osmolar buffer was added to a 10 mg/ml solution of gemcitabine (GEMZAR) in deionized water. A 500 it aliquot was treated by electroporation using a GENE PULSER II instrument (BIO-RAD) in a 4 mm gap cuvette (settings: 96 μF, 220 V, 20 milliseconds).

Another aliquot was sonicated with a BRANSON W-250 SONIFIER (I=20, Cycle=50%, 5 minutes at 4° C.). Then, unincorporated gemcitabine was removed using AMICON (Millipore) concentrators (pore=10 kDa) washing three times with 1 ml of Hank's balanced salt solution (HBSS) each time. Flow-through, wash fractions and recovered particle suspension were analyzed spectrophotometrically by measuring absorbance at 260 nm and the concentration of gemcitabine was calculated using a standard curve. The calculated amount of entrapped gemcitabine was about 10% in the electroporated sample and about 5% in the sonicated sample.

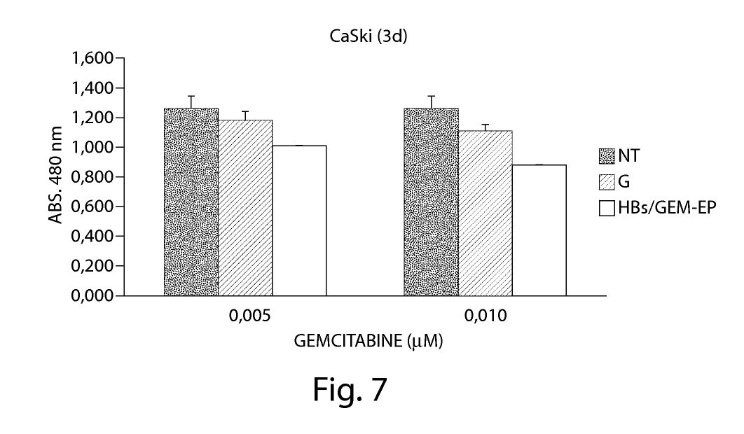

A cervical cancer cell line (CaSki) was used for testing the cytotoxicity of entrapped gemcitabine as compared to free gemcitabine. CaSki cells were either left untreated (NT), or were treated with a single dose of Gemcitabine (G), or with Gemcitabine-loaded nanoparticles (HBs/Gem-EP) and incubated for 3 days. The bar graph of FIG. 7 shows cell viability for two very low concentrations of drug, 0.005 micromolar and 0.010 micromolar. Cell viability was measured with the XTT kit from Roche following manufacturer's instructions. The HBs/Gem-EP treated sample showed more efficient killing of cells compared to free Gemcitabine (G).

FIG. 7 depicts a bar graph showing a cell viability assay in vitro. Cervical cancer cells (CaSki cell line) were either non-treated (NT) or treated with gemcitabine encapsulated into HBsΔ⁹⁸ particles by electroporation (HBs/Gem-EP) or un-encapsulated gemcitabine (G).

Each of the foregoing patents, patent applications and references that are recited in this application are herein incorporated in their entirety by reference. Having thus described several aspects of embodiments of this invention, it is to be appreciated various alterations, modifications, and improvements will readily occur to those skilled in the art in view of the teachings set forth herein. Such alterations, modifications, and improvements are intended to be part of this disclosure, and are intended to be within the spirit and scope of the invention. Accordingly, the foregoing description and drawings are by way of example only.

What is claimed is:

1. An isolated hollow nanoparticle comprising a Hepatitis B Surface Antigen S protein domain having a truncation, the truncation comprising an amino-terminal deletion of at least one transmembrane domain.

2. A membrane-enclosed vesicle, wherein the membrane comprises the Hepatitis B Surface Antigen S protein domain of claim 1.

3. A substance delivery system for targeted delivery, the system comprising: the isolated hollow nanoparticle of claim 1.

4. The targeted substance delivery system of claim 3, wherein the truncated Hepatitis B Surface Antigen S protein domain further comprises one or more deletions or mutations within the ‘a’ determinant region reducing the immunogenicity of the ‘a’ determinant region, one or more deletions or mutations reducing the B cell mediated immunogenicity of the S protein domain, and/or one or more deletions or mutations reducing the T cell mediated immunogenicity of the S protein domain.

5. The targeted substance delivery system of claim 4, wherein the truncated Hepatitis B Surface Antigen S protein domain further comprises one or more additional deletions or mutations outside the ‘a’ determinant region.

6-7. (canceled)

8. The isolated hollow nanoparticle of claim 1, wherein the nanoparticle is between 15 nm and 30 nm in size.

9. (canceled)

10. The targeted substance delivery system of claim 3, wherein the substance is a nucleic acid, a protein, a small molecule or an imaging agent.

11. (canceled)

12. (canceled)

13-16. (canceled)

17. The targeted substance delivery system of claim 10, wherein the nucleic acid is a siRNA, microRNA, antisense RNA, DNA molecule or gene delivery vehicle.

18-24. (canceled)

25. The isolated hollow nanoparticle of claim 2, wherein the membrane further comprises a peptide or non-peptide targeting molecule, wherein the non-peptide targeting molecule is an aptamer, and the peptide targeting molecule is an antibody or a peptide-ligand.

26-27. (canceled)

28. The isolated hollow nanoparticle of any one of claim 25, wherein the peptide targeting molecule is fused to the S protein domain.

29. The isolated hollow nanoparticle of claim 25, wherein the targeting molecule directs the nanoparticle to a tumor cell or to a liver cell.

30-36. (canceled)

37. The isolated hollow nanoparticle of claim 1, wherein the truncation of the Hepatitis B Surface Antigen S protein domain further comprises an amino-terminal deletion of an additional, second transmembrane domain.

38. An isolated Hepatitis B Surface Antigen S domain protein having a truncation, the truncation comprising an amino-terminal deletion of at least one transmembrane domain, wherein the truncated Hepatitis B Surface Antigen S protein domain further comprises:
(a) an amino-terminal deletion of an additional, second transmembrane domain;
(b) a deletion or mutation that reduces B cell mediated immunogenicity of the truncated S domain protein;
(c) a deletion or mutation that reduces T cell mediated immunogenicity of the truncated S domain protein;
(d) a targeting domain;
(e) a purification tag; or
(f) an identification tag.

39-40. (canceled)

41. An isolated truncated Hepatitis B Surface Antigen S domain protein having an amino acid sequence of SEQ ID NO: 1-17, 57, 58, 60, 63, 67-69, and 73-84.

42-45. (canceled)

46. The isolated truncated Hepatitis B Surface Antigen S domain protein of claim 38, wherein the targeting domain is an integrin receptor binding domain, an epithelial growth factor (EGF) receptor binding domain, fibroblast growth factor (FGF) receptor binding domain, a pre-S1 binding domain or an albumin binding domain.

47-55. (canceled)

56. An isolated DNA encoding a truncated Hepatitis B Surface Antigen S domain protein, wherein the DNA has a nucleotide sequence of SEQ ID NO: 18-51 and 85-118.

57. A host cell comprising the isolated DNA of claim 56.

58. (canceled)

59. A pharmaceutical composition comprising: the targeted substance delivery system of claim 10 and a pharmaceutically acceptable carrier.

60-68. (canceled)

69. A method of treating a subject having an adverse condition, the method comprising administering to the subject the composition of claim 59 in an amount effective to treat the condition.

70. A method of diagnosing a subject having an adverse condition or at risk of developing an adverse condition, the method comprising administering to the subject the composition of claim 59 in an amount effective to diagnose the adverse condition.

71. The method of claim 69, wherein the adverse condition is liver cancer or liver disease.

72. (canceled)
